(19) United States
(12) Patent Application Publication
(10) Pub. No.: US 2017/0283862 A1
(43) Pub. Date: Oct. 5, 2017

(54) GENETIC TESTING FOR PREDICTING RESISTANCE OF KLEBSIELLA SPECIES AGAINST ANTIMICROBIAL AGENTS

(71) Applicant: Curetis GmbH, Holzgerlingen (DE)

(72) Inventors: Andreas Keller, Puttlingen (DE); Susanne Schmolke, Munchen (DE); Cord Friedrich Stahler, Hirschberg an der Bergstrasse (DE); Christina Backes, Saarbrucken (DE)

(21) Appl. No.: 15/512,166

(22) PCT Filed: Aug. 6, 2015
(86) PCT No.: PCT/EP2015/068133

§ 371 Date: Mar. 17, 2017

(30) Foreign Application Priority Data
Sep. 25, 2014 (EP) 14186404.1

(51) Int. Cl. C12Q 1/68; G06F 19/24; G06F 19/22

(52) U.S. Cl. CPC C12Q 1/689; G06F 19/22; G06F 19/24; C12Q 2600/156; G06F 19/345

(57) ABSTRACT

The invention relates to a method of determining an infection of a patient with Klebsiella species potentially resistant to antimicrobial drug treatment by detecting mutations in the genes, parC, KPN 01607, gyrA, KPN 02451, baeR, aceF, ybgH, ynjE, KPN 01951, KPN 01961, KPN 02114, mhpA, KPN 02128, KPN 02144, KPN 02149, ydiJ, btuE, oppC, pth, KPN 02298, KPN 02302, dadA, yoaA, ftn, cbl, hisB, yegQ, yehY, KPN 02580, yejH, KPN 02621, yfaW, KPN 02170, KPN 02025, livG, livM, livH, fliY, yedQ, abgB, treA, baeS, KPN 02399, ydcR, anmK, ccmF, KPN 02440, KPN 02540, KPN 01752, and KPN 04195, and/or KOX 26125, KOX 13365, KOX 16735, KOX 25695, KOX 12270, and KOX 15055; a method of selecting a treatment of a patient suffering from an antibiotic resistant Klebsiella infection; and a method of determining an antibiotic resistance profile for bacterial microorganisms of Klebsiella species, as well as computer program products used in these methods. In an exemplary method, a sample is used for molecular testing and then a molecular fingerprint is taken. The result is then compared to a reference library and the result is reported.

The present invention relates to a method of determining an infection of a patient with Klebsiella species potentially resistant to antimicrobial drug treatment, a method of selecting a treatment of a patient suffering from an infection with a potentially resistant Klebsiella strain, and a method of determining an antimicrobial drug, e.g. antibiotic, resistance profile for bacterial microorganisms of Klebsiella species, as well as computer program products used in these methods.

Further, the invention relates to a method of determining an antibiotic resistance profile for E. coli and Klebsiella pneumoniae and to a method of determining the resistance of E. coli to Klebsiella pneumoniae an antibiotic drug.

Antibiotic resistance is a form of drug resistance whereby a sub-population of a microorganism, e.g. a strain of a bacterial species, can survive and multiply despite exposure to an antibiotic drug. It is a serious and health concern for the individual patient as well as a major public health issue. Timely treatment of a bacterial infection requires the analysis of clinical isolates obtained from patients with regard to antibiotic resistance, in order to select an efficacious therapy. Generally, for this purpose an association of the identified resistance with a certain microorganism (i.e. ID) is necessary.

Antibacterial drug resistance (ADR) represents a major health burden. According to the World Health Organization's antimicrobial resistance global report on surveillance, ADR leads to 25,000 deaths per year in Europe and 23,000 deaths per year in the US. In Europe, 2.5 million extra hospital days lead to societal cost of 1.5 billion euro. In the US, the direct cost of 2 million illnesses leads to 20 billion dollar direct cost. The overall cost is estimated to be substantially higher, reducing the gross domestic product (GDP) by up to 1.6%.

Klebsiella species are Gram-negative rods belonging to the family of Enterobacteriaceae. K pneumoniae and K oxytoca are the 2 members of this genus responsible for most human infections. The spectrum of clinical syndromes includes pneumonia, bacteremia, thrombophlebitis, urinary tract infection, diarrhea, upper respiratory tract infection, wound infection, and meningitis. Infections with K pneumoniae are particularly common in hospitals among vulnerable individuals such as pre-term infants and patients with impaired immune-systems, and those receiving advanced medical care. Mortality rates for K pneumoniae hospital-acquired pneumonia depend on the severity of the underling condition, and can exceed 50% in vulnerable patients, even when treated with appropriate antibacterial drugs.

According to the 2014 WHO Report ‘Antimicrobial Resistance: Global Report on Surveillance’ a majority of countries reported more than 30% resistance in K pneumoniae against third-generation cephalosporins (commonly used to treat severe infections in hospitals) meaning that treatment for verified or suspected in K pneumoniae infections has to rely on carbapenems involving higher costs and the risk for further expansion of carbapenem resistant strains. The report found an alarming rate of the latter which leaves very few if any alternative treatment options in some patient groups.

The considerable and ongoing increase of infections caused by multi-drug resistant pathogens represents a major threat especially in a hospital setting and for those patients with critical illness. The development of new drugs is a long and expensive venture, and stagnated in the last years despite increasing investments in research and development.

Abundant prescribing of broad-spectrum antibiotics promotes the development of multi-drug resistance in bacteria. Also, the application of antibiotics with partial susceptibility increases the likelihood that bacterial strains evolve with increasing resistance due to the imposed selection pressure. Hence, clinically applicable methods are needed for a more careful selection of antibiotics to quickly stratify patients and provide them with the optimal therapy. Moreover, improved knowledge on genetic drug resistance mechanisms may lead to novel drugs.

In general the mechanisms for resistance of bacteria against antimicrobial treatments rely to a very substantial part on the organism's genetics. The respective genes or molecular mechanisms are either encoded in the genome of the bacteria or on plasmids that can be interchanged between different bacteria. The most common resistance mechanisms include:
1) Efflux pumps are high-affinity reverse transport systems located in the membrane that transports the antibiotic out of the cell, e.g. resistance to tetracycline.
2) Specific enzymes modify the antibiotic in a way that it loses its activity. In the case of streptomycin, the antibiotic is chemically modified so that it will no longer bind to the ribosome to block protein synthesis.
3) An enzyme is produced that degrades the antibiotic, thereby inactivating it. For example, the penicillinases are a group of beta-lactamase enzymes that cleave the beta lactam ring of the penicillin molecule.

In addition, some pathogens show natural resistance against drugs. For example, an organism can lack a transport system for an antibiotic or the target of the antibiotic molecule is not present in the organism.

Pathogens that are in principle susceptible to drugs can become resistant by modification of existing genetic material (e.g. spontaneous mutations for antibiotic resistance, happening in a frequency of one in about 100 mio bacteria in an infection) or the acquisition of new genetic material from another source. One example is horizontal gene transfer, a process where genetic material contained in small packets of DNA can be transferred between individual bacteria of the same species or even between different species. Horizontal gene transfer may happen by transduction, transformation or conjugation.

Generally, testing for susceptibility/resistance to antimicrobial agents is performed by culturing organisms in different concentration of these agents.

Currently, resistance/susceptibility testing is carried out by obtaining a culture of the suspicious bacteria, subjecting it to different antibiotic drug protocols and determining in which cases bacteria do not grow in the presence of a certain substance. In this case the bacteria are not resistant (i.e. susceptible to the antibiotic drug) and the therapy can be administered to the respective patients.

In brief, agar plates are inoculated with patient sample (e.g. urine, sputum, blood, stool) overnight. On the next day individual colonies are used for identification of organisms, either by culturing or using mass spectroscopy. Based on the identity of organisms new plates containing increasing concentration of drugs used for the treatment of these organisms are inoculated and grown for additional 12-24 hours. The lowest drug concentration which inhibits growth (minimal inhibitory concentration—MIC) is used to determine susceptibility/resistance for tested drugs. The process takes at least 2 to 3 working days during which the patient is treated empirically. A significant reduction of time-to-result is needed especially in patients with life-threatening disease and to overcome the widespread misuse of antibiotics.

Recent technological advances promise to improve identification of bacteria. Recent developments include PCR based test kits for fast bacterial identification (e.g. Biomerieux Biofire Tests, Curetis Unyvero Tests). With these test the detection of selected resistance loci is possible for a very limited number of drugs, but no correlation to culture based AST is given. Mass spectroscopy is increasingly used for identification of pathogens in clinical samples (e.g. Bruker Biotyper), and research is ongoing to establish methods for the detection of susceptibility/resistance against antibiotics. While e.g. Matrix-Assisted Laser Desorption Ionization-Time of Flight (MALDI TOF) Mass Spectrometry is successfully applied for detection of bacteria, the application of this technique for resistance testing is still in its infancy, though.

Significant improvements in genotyping technologies promoted a new class of genetic antimicrobial susceptibility tests. Already in 2002, Beerenwinkel and co-workers investigated the diversity of HIV-1 drug resistance based on the viral genome, showing a good performance of their genetic approach on 471 different clinical HIV isolates with error rates below 15% (Beerenwinkel, N. et al. Diversity and complexity of HIV-1 drug resistance: a bioinformatics approach to predicting phenotype from genotype. Proc Natl Acad Sci USA 99, 8271-6 (2002)). In line with these developments and driven by the progress of Next-Generation Sequencing (NGS), the genetic basis of resistance mechanisms for different bacteria is currently explored. Currently, different gram-negative and gram-positive bacteria such as S. aureus, M. tuberculosis, S. pneumoniae, or K. pneumoniae are analyzed.

For some drugs such it is known that at least two targets are addressed, e.g. in case of Ciprofloxacin (drug bank ID 00537; http://www.drugbank.ca/drugs/DB00537) targets include DNA Topoisomerase IV, DNA Topoisomerase II and DNA Gyrase. It can be expected that this is also the case for other drugs although the respective secondary targets have not been identified yet. In case of a common regulation, both relevant genetic sites would naturally show a co-correlation or redundancy.

It is known that drug resistance can be associated with genetic polymorphisms. This holds for viruses, where resistance testing is established clinical practice (e.g. HIV genotyping). More recently, it has been shown that resistance has also genetic causes in bacteria and even higher organisms, such as humans where tumors resistance against certain cytostatic agents can be linked to genomic mutations.

Wozniak et al. (BMC Genomics 2012, 13(Suppl 7):S23) disclose genetic determinants of drug resistance in Staphylococcus aureus based on genotype and phenotype data. Stoesser et al. disclose prediction of antimicrobial susceptibilities for Escherichia coli and Klebsiella pneumoniae isolates using whole genomic sequence data (J Antimicrob Chemother 2013; 68: 2234-2244).

Chewapreecha et al (Chewapreecha et al (2014) Comprehensive Identification of single nucleotid polymorphisms associated with beta-lactam resistance within pneumococcal mosaic genes. PLoS Genet 10(8): e1004547) used a comparable approach to identify mutations in gram-positive Streptococcus Pneumonia.

The fast and accurate detection of infections with Klebsiella species and the prediction of response to anti-microbial therapy represent a high unmet clinical need. Further, to personalize current therapies and to develop novel drugs it is crucial to understand the genetic diversity of pathogenic bacteria.

This need is addressed by the present invention.

### SUMMARY OF THE INVENTION

The present inventors addressed this need by carrying out whole genome sequencing of a large cohort of Klebsiella clinical isolates and comparing the genetic mutation profile to classical culture based antimicrobial susceptibility testing with the goal to develop a test which can be used to detect bacterial susceptibility/resistance against antimicrobial drugs using molecular testing.

The inventors performed extensive studies on the genome of bacteria of Klebsiella species either susceptible or resistant to antimicrobial, e.g. antibiotic, drugs. Based on this information, it is now possible to provide a detailed analysis on the resistance pattern of Klebsiella strains based on individual genes or mutations on a nucleotide level. This analysis involves the identification of a resistance against individual antimicrobial, e.g. antibiotic, drugs as well as clusters of them. This allows not only for the determination of a resistance to a single antimicrobial, e.g. antibiotic, drug, but also to groups of antimicrobial drugs, e.g. antibiotics such as lactam or quinolone antibiotics, or even to all relevant antibiotic drugs.

Therefore, the present invention will considerably facilitate the selection of an appropriate antimicrobial, e.g. antibiotic, drug for the treatment of a Klebsiella infection in a patient and thus will largely improve the quality of diagnosis and treatment.

According to a first aspect, the present invention discloses a diagnostic method of determining an infection of a patient with Klebsiella species potentially resistant to antimicrobial drug treatment, which can be also described as a method of determining an antimicrobial drug, e.g. antibiotic, resistant Klebsiella infection of a patient, comprising the steps of:

a) obtaining or providing a sample containing or suspected of containing at least one Klebsiella species from the patient;
b) determining the presence of at least one mutation in at least two genes from the group of genes listed in Table 1a and/or Table 1b, or Table 2a and/or Table 2b below, wherein the presence of said at least two mutations is indicative of an infection with an antimicrobial drug resistant, e.g. antibiotic resistant, Klebsiella strain in said patient.

An infection of a patient with Klebsiella species potentially resistant to antimicrobial drug treatment herein means an infection of a patient with Klebsiella species wherein it is unclear if the Klebsiella species is susceptible to treatment with a specific antimicrobial drug or if it is resistant to the antimicrobial drug.

TABLE 1a
List of genes, particularly for Klebsiella pneumoniae

| | | | | |
|---|---|---|---|---|
| parC | KPN_01607 | gyrA | KPN_02451 | baeR |
| aceF | ybgH | ynjE | KPN_01951 | KPN_01961 |
| KPN_02114 | mhpA | KPN_02128 | KPN_02144 | KPN_02149 |
| ydiJ | btuE | oppC | pth | KPN_02298 |
| KPN_02302 | dadA | yoaA | ftn | cbl |
| hisB | yegQ | yehY | KPN_02580 | yejH |
| KPN_02621 | yfaW | KPN_02170 | KPN_02025 | livG |
| livM | livH | fliY | yedQ | abgB |
| treA | baeS | KPN_02399 | ydcR | anmK |
| ccmF | KPN_02440 | KPN_02540 | KPN_01752 | KPN_04195 |

TABLE 1b
List of genes, particularly for Klebsiella oxytoca

| | | |
|---|---|---|
| KOX_26125 | KOX_13365 | KOX_16735 |
| KOX_25695 | KOX_12270 | KOX_15055 |

In step b) above, as well as corresponding steps, at least one mutation in at least two genes is determined, so that in total at least two mutations are determined, wherein the two mutations are in different genes.

TABLE 2a
List of genes, particularly for Klebsiella pneumoniae

| | | | | |
|---|---|---|---|---|
| parC | KPN_01607 | gyrA | KPN_02451 | baeR |
| aceF | ybgH | ynjE | KPN_01951 | KPN_01961 |
| KPN_02114 | mhpA | KPN_02128 | KPN_02144 | KPN_02149 |
| ydiJ | btuE | oppC | pth | KPN_02298 |
| KPN_02302 | dadA | yoaA | ftn | cbl |
| hisB | yegQ | yehY | KPN_02580 | yejH |
| KPN_02621 | yfaW | KPN_02170 | KPN_02025 | livG |
| livM | livH | fliY | yedQ | abgB |
| treA | baeS | KPN_02399 | ydcR | anmK |
| ccmF | KPN_02440 | KPN_02540 | KPN_01752 | KPN_04195 |

TABLE 2b
List of genes, particularly for Klebsiella oxytoca

| | | |
|---|---|---|
| KOX_26125 | KOX_13365 | KOX_16735 |
| KOX_25695 | KOX_12270 | KOX_15055 |

According to a second aspect, the present invention relates to a method of selecting a treatment of a patient suffering from an infection with a potentially resistant Klebsiella strain, e.g. from an antimicrobial drug, e.g. antibiotic, resistant Klebsiella infection, comprising the steps of:

a) obtaining or providing a sample containing or suspected of containing at least one Klebsiella species from the patient;
b) determining the presence of at least one mutation in at least two genes from the group of genes listed in Table 1 or Table 2 above, wherein the presence of said at least two mutations is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella infection.

A third aspect of the present invention relates to a method of determining an antimicrobial drug, e.g. antibiotic, resistance profile for bacterial microorganisms of Klebsiella species, comprising:

obtaining or providing a first data set of gene sequences of a plurality of clinical isolates of Klebsiella species;
providing a second data set of antimicrobial drug, e.g. antibiotic, resistance of the plurality of clinical isolates of Klebsiella species;
aligning the gene sequences of the first data set to at least one, preferably one, reference genome of Klebsiella, and/or assembling the gene sequence of the first data set, at least in part;
analyzing the gene sequences of the first data set for genetic variants to obtain a third data set of genetic variants; correlating the third data set with the second data set and statistically analyzing the correlation; and
determining the genetic sites in the genome of Klebsiella associated with antimicrobial drug, e.g. antibiotic, resistance.

In addition, the present invention relates in a fourth aspect to a method of determining an antimicrobial drug, e.g. antibiotic, resistance profile for a bacterial microorganism belonging to the species Klebsiella comprising the steps of

a) obtaining or providing a sample containing or suspected of containing the bacterial microorganism;
b) determining the presence of a mutation in at least one gene of the bacterial microorganism as determined by the method according to the third aspect of the present invention;
wherein the presence of a mutation is indicative of a resistance to an antimicrobial, e.g. antibiotic, drug.

Furthermore, the present invention discloses in a fifth aspect a diagnostic method of determining an infection of a patient with Klebsiella species potentially resistant to antimicrobial drug treatment, which can, like in the first aspect, also be described as method of determining an antimicrobial drug, e.g. antibiotic, resistant Klebsiella infection of a patient, comprising the steps of:

a) obtaining or providing a sample containing or suspected of containing a bacterial microorganism belonging to the species Klebsiella from the patient;
b) determining the presence of at least one mutation in at least one gene of the bacterial microorganism belonging to the species Klebsiella as determined by the method according to the third aspect of the present invention, wherein the presence of said at least one mutation is indicative of an antimicrobial drug, e.g. antibiotic, resistant Klebsiella infection in said patient.

Also disclosed is in a sixth aspect a method of selecting a treatment of a patient suffering from an infection with a potentially resistant Klebsiella strain, e.g. from an antimicrobial drug, e.g. antibiotic, resistant Klebsiella infection, comprising the steps of:

a) obtaining or providing a sample containing or suspected of containing a bacterial microorganism belonging to the species Klebsiella from the patient;
b) determining the presence of at least one mutation in at least one gene of the bacterial microorganism belonging to the species Klebsiella as determined by the method according to the third aspect of the present invention, wherein the presence of said at least one mutation is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella infection.

A seventh aspect of the present invention relates to a method of acquiring, respectively determining, an antimicrobial drug, e.g. antibiotic, resistance profile for a bacterial microorganisms of Klebsiella species, comprising:

obtaining or providing a first data set of gene sequences of a clinical isolate of Klebsiella species;
providing a second data set of antimicrobial drug, e.g. antibiotic, resistance of a plurality of clinical isolates of Klebsiella species;
aligning the gene sequences of the first data set to at least one, preferably one, reference genome of Klebsiella, and/or assembling the gene sequence of the first data set, at least in part;
analyzing the gene sequences of the first data set for genetic variants to obtain a third data set of genetic variants of the first data set;
correlating the third data set with the second data set and statistically analyzing the correlation; and
determining the genetic sites in the genome of Klebsiella of the first data set associated with antimicrobial drug, e.g. antibiotic, resistance.

According to an eighth aspect, the present invention discloses a computer program product comprising executable instructions which, when executed, perform a method according to the third, fourth, fifth, sixth or seventh aspect of the present invention.

Further aspects and embodiments of the invention are disclosed in the dependent claims and can be taken from the following description, figures and examples, without being limited thereto.

### FIGURES

The enclosed drawings should illustrate embodiments of the present invention and convey a further understanding thereof. In connection with the description they serve as explanation of concepts and principles of the invention. Other embodiments and many of the stated advantages can be derived in relation to the drawings. The elements of the drawings are not necessarily to scale towards each other. Identical, functionally equivalent and acting equal features and components are denoted in the figures of the drawings with the same reference numbers, unless noted otherwise.

FIG. 1 shows schematically a read-out concept for a diagnostic test according to a method of the present invention.

FIG. 2 shows an exemplary contingency table for the computation of the Fisher's exact test and the measures accuracy, sensitivity, specificity, positive predictive value (PPV), and negative predictive value (NPV) in the Examples, particularly example 2. Numbers are given for amino acid exchange S83L (GyrA) and Ciprofloxacin in E. coli.

FIG. 3 shows an overview of mean MIC values for Ciprofloxacin for E. coli samples having no mutation in GyrA (S83, D87) and ParC (S80), either one mutation in GyrA and not ParC, both mutations in GyrA and not ParC, or all three mutations in the Examples, particularly example 2.

FIG. 4 shows the following regarding the Examples, particularly example 2: Panel A: bar chart of E. coli genes with highest number of significant sites. Panel B. bar chart detailing the genes with highest number of sites correlated to at least 3 drugs. Panel C. Scatter plot showing for each gene the number of significant sites correlated with at least 3 drugs as function of total number of significant sites in the gene. Panel D. Along gene plot for yjgN. The significant sites along the genetic sequence are presented as dots, the y-axis shows the number of drug classes significant for the respective site. Below, a so called snake plot of the trans-membrane protein is shown, the affected amino acids are colored.

FIG. 5 shows the following regarding the Examples, particularly example 2: Panel A: network diagram showing drugs as rectangles and E. coli genes with higher or lower coverage if resistance for the respective drug is shown as circles. Panel B and C: two example along-chromosome plots.

### DETAILED DESCRIPTION OF THE PRESENT INVENTION

### Definitions

Unless defined otherwise, technical and scientific terms used herein have the same meaning as commonly understood by one of ordinary skill in the art to which this invention belongs.

An “antimicrobial drug” in the present invention refers to a group of drugs that includes antibiotics, antifungals, antiprotozoals, and antivirals. According to certain embodiments, the antimicrobial drug is an antibiotic.

The term “nucleic acid molecule” refers to a polynucleotide molecule having a defined sequence. It comprises DNA molecules, RNA molecules, nucleotide analog molecules and combinations and derivatives thereof, such as DNA molecules or RNA molecules with incorporated nucleotide analogs or cDNA.

The term “nucleic acid sequence information” relates to an information which can be derived from the sequence of a nucleic acid molecule, such as the sequence itself or a variation in the sequence as compared to a reference sequence.

The term “mutation” relates to a variation in the sequence as compared to a reference sequence. Such a reference sequence can be a sequence determined in a predominant wild type organism or a reference organism, e.g. a defined and known bacterial strain or substrain. A mutation is for example a deletion of one or multiple nucleotides, an insertion of one or multiple nucleotides, or substitution of one or multiple nucleotides, duplication of one or a sequence of multiple nucleotides, translocation of one or a sequence of multiple nucleotides, and, in particular, a single nucleotide polymorphism (SNP).

In the context of the present invention a “sample” is a sample which comprises at least one nucleic acid molecule from a bacterial microorganism. Examples for samples are: cells, tissue, body fluids, biopsy specimens, blood, urine, saliva, sputum, plasma, serum, cell culture supernatant, swab sample and others. According to certain embodiments, the sample is a patient sample (clinical isolate).

New and highly efficient methods of sequencing nucleic acids referred to as next generation sequencing have opened the possibility of large scale genomic analysis. The term “next generation sequencing” or “high throughput sequencing” refers to high-throughput sequencing technologies that parallelize the sequencing process, producing thousands or millions of sequences at once. Examples include Massively Parallel Signature Sequencing (MPSS), Polony sequencing, 454 pyrosequencing, Illumina (Solexa) sequencing, SOLiD sequencing, Ion semiconductor sequencing, DNA nanoball sequencing, Helioscope™ single molecule sequencing, Single Molecule SMRT™ sequencing, Single Molecule real time (RNAP) sequencing, Nanopore DNA sequencing, Sequencing By Hybridization, Amplicon Sequencing, GnuBio.

Within the present description the term “microorganism” comprises the term microbe. The type of microorganism is not particularly restricted, unless noted otherwise or obvious, and, for example, comprises bacteria, viruses, fungi, microscopic algae and protozoa, as well as combinations thereof. According to certain aspects, it refers to one or more Klebsiella species, particularly Klebsiella pneumoniae and/or Klebsiella oxytoca.

A reference to a microorganism or microorganisms in the present description comprises a reference to one microorganism as well a plurality of microorganisms, e.g. two, three, four, five, six or more microorganisms.

A vertebrate within the present invention refers to animals having a vertebrae, which includes mammals—including humans, birds, reptiles, amphibians and fishes. The present invention thus is not only suitable for human medicine, but also for veterinary medicine.

According to certain embodiments, the patient in the present methods is a vertebrate, more preferably a mammal and most preferred a human patient.

Before the invention is described in exemplary detail, it is to be understood that this invention is not limited to the particular component parts of the process steps of the methods described herein as such methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. For example, the term “a” as used herein can be understood as one single entity or in the meaning of “one or more” entities. It is also to be understood that plural forms include singular and/or plural referents unless the context clearly dictates otherwise. It is moreover to be understood that, in case parameter ranges are given which are delimited by numeric values, the ranges are deemed to include these limitation values.

Regarding the dosage of the antimicrobial, e.g. antibiotic, drugs, it is referred to the established principles of pharmacology in human and veterinary medicine. For example, Forth, Henschler, Rummel “Allgemeine und spezielle Pharmakologie und Toxikologie”, 9th edition, 2005 might be used as a guideline. Regarding the formulation of a ready-to-use medicament, reference is made to “Remington, The Science and Practice of Pharmacy”, 22^{nd} edition, 2013.

Assembling of a gene sequence can be carried out by any known method and is not particularly limited.

According to certain embodiments, mutations that were found using alignments can also be compared or matched with alignment-free methods, e.g. for detecting single base exchanges, for example based on contigs that were found by assemblies. For example, reads obtained from sequencing can be assembled to contigs and the contigs can be compared to each other.

According to a first aspect, the present invention relates to a diagnostic method of determining an infection of a patient with Klebsiella species, particularly Klebsiella pneumoniae and/or Klebsiella oxytoca, potentially resistant to antimicrobial drug treatment, which can also be described as method of determining an antimicrobial drug, e.g. antibiotic, resistant Klebsiella, particularly Klebsiella pneumoniae and/or Klebsiella oxytoca, infection of a patient, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least two genes from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, wherein the presence of said at least two mutations is indicative of an infection with an antimicrobial, e.g. antibiotic, resistant Klebsiella, particularly Klebsiella pneumoniae and/or Klebsiella oxytoca, strain in said patient.

According to certain embodiments, the method of the first aspect relates to a diagnostic method of determining an infection of a patient with Klebsiella species, particularly Klebsiella pneumoniae, potentially resistant to antimicrobial drug treatment, which can also be described as method of determining an antimicrobial drug, e.g. antibiotic, resistant Klebsiella, particularly Klebsiella pneumoniae, infection of a patient, comprising the steps of:

a) obtaining or providing a sample containing or suspected of containing at least one Klebsiella species, particularly Klebsiella pneumoniae, from the patient;
b) determining the presence of at least one mutation in at least two genes from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, wherein the presence of said at least two mutations is indicative of an infection with an antimicrobial, e.g. antibiotic, resistant Klebsiella, particularly Klebsiella pneumoniae, strain in said patient.

According to certain embodiments, the method of the first aspect relates to a diagnostic method of determining an infection of a patient with Klebsiella species, particularly Klebsiella oxytoca, potentially resistant to antimicrobial drug treatment, which can also be described as method of determining an antimicrobial drug, e.g. antibiotic, resistant Klebsiella, particularly Klebsiella oxytoca, infection of a patient, comprising the steps of:

a) obtaining or providing a sample containing or suspected of containing at least one Klebsiella species, particularly Klebsiella oxytoca, from the patient;
b) determining the presence of at least one mutation in at least two genes from the group of genes consisting of KOX_26125, KOX_13365, KOX_16735, KOX_25695, KOX_12270, and KOX_15055, wherein the presence of said at least two mutations is indicative of an infection with an antimicrobial, e.g. antibiotic, resistant Klebsiella, particularly Klebsiella oxytoca, strain in said patient.

In this method, as well as the other methods of the invention, the sample can be provided or obtained in any way, preferably non-invasive, and can be e.g. provided as an in vitro sample or prepared as in vitro sample.

According to certain aspects, mutations in at least two, three, four, five, six, seven, eight, nine or ten genes are determined in any of the methods of the present invention, e.g. in at least two genes or in at least three genes. Instead of testing only single genes or mutants, a combination of several variant positions can improve the prediction accuracy and further reduce false positive findings that are influenced by other factors. Therefore, it is in particular preferred to determine the presence of a mutation in 2, 3, 4, 5, 6, 7, 8 or 9 (or more) genes selected from Table 1 or 2.

For the above genes, particularly for K. pneumoniae, i.e. the genes also denoted in Tables 1a and 2a, the highest probability of a resistance to at least one antimicrobial drug, e.g. antibiotic, could be observed, with p-values smaller than 10⁻⁹⁰, particularly smaller than 10⁻¹⁰⁰, particularly smaller than 10⁻¹¹⁰, indicating the high significance of the values (n=1176; a=0.05). For the above genes, particularly for K. oxytoca, i.e. the genes also denoted in Tables 1b and 2b, the highest probability of a resistance to at least one antimicrobial drug, e.g. antibiotic, could be observed, with p-values smaller than 10⁻³⁰, particularly smaller than 10⁻⁴⁰, indicating the high significance of the values (n=400; α=0.05).

Details regarding Tables 1a and 2a can be taken from Tables 3a and 4a, 4b, 4c disclosed in the Examples, and details regarding Tables 1b and 2b can be taken from Tables 3b and 4d, 4e, 4f disclosed in the Examples.

Having at least two genes with mutations determined, a high probability of an antimicrobial drug, e.g. antibiotic, resistance could be determined. The genes in Tables 1a and 1b thereby represent the 50 best genes for which a mutation was observed in the genomes of Klebsiella species, particularly Klebsiella pneumoniae and/or Klebsiella oxytoca, whereas the genes in Tables 2a and 2b represent the best genes for which a cross-correlation could be observed for the antimicrobial drug, e.g. antibiotic, susceptibility testing for Klebsiella species, particularly Klebsiella pneumoniae and/or Klebsiella oxytoca, as described below.

According to certain embodiments, the obtaining or providing a sample containing or suspected of containing at least one Klebsiella species, particularly Klebsiella pneumoniae and/or Klebsiella oxytoca, from the patient in this method—as well as the other methods of the invention—can comprise the following:

A sample of a vertebrate, e.g. a human, e.g. is provided or obtained and nucleic acid sequences, e.g. DNA or RNA sequences, are recorded by a known method for recording nucleic acid, which is not particularly limited. For example, nucleic acid can be recorded by a sequencing method, wherein any sequencing method is appropriate, particularly sequencing methods wherein a multitude of sample components, as e.g. in a blood sample, can be analyzed for nucleic acids and/or nucleic acid fragments and/or parts thereof contained therein in a short period of time, including the nucleic acids and/or nucleic acid fragments and/or parts thereof of at least one microorganism of interest, particularly of at least one Klebsiella species, particularly Klebsiella pneumoniae and/or Klebsiella oxytoca. For example, sequencing can be carried out using polymerase chain reaction (PCR), particularly multiplex PCR, or high throughput sequencing or next generation sequencing, preferably using high-throughput sequencing. For sequencing, preferably an in vitro sample is used.

The data obtained by the sequencing can be in any format, and can then be used to identify the nucleic acids, and thus genes, of the microorganism, e.g. of Klebsiella species, particularly Klebsiella pneumoniae and/or Klebsiella oxytoca, to be identified, by known methods, e.g. fingerprinting methods, comparing genomes and/or aligning to at least one, or more, genomes of one or more species of the microorganism of interest, i.e. a reference genome, etc., forming a third data set of aligned genes for a Klebsiella species, particularly Klebsiella pneumoniae and/or Klebsiella oxytoca—discarding additional data from other sources, e.g. the vertebrate. Reference genomes are not particularly limited and can be taken from several databases. Depending on the microorganism, different reference genomes or more than one reference genomes can be used for aligning. Using the reference genome—as well as also the data from the genomes of the other species, e.g. Klebsiella species, particularly Klebsiella pneumoniae and/or Klebsiella oxytoca—mutations in the genes for each species and for the whole multitude of samples of different species, e.g. Klebsiella species, particularly Klebsiella pneumoniae and/or Klebsiella oxytoca, can be obtained.

For example, it is useful in genome-wide association studies to reference the points of interest, e.g. mutations, to one constant reference for enhanced standardization. In case of the human with a high consistency of the genome and 99% identical sequences among individuals this is easy and represents the standard, as corresponding reference genomes are available in databases. In case of organisms that trigger infectious diseases (e.g. bacteria and viruses) this is much more difficult, though. One possibility is to fall back on a virtual pan genome which contains all sequences of a certain genus. A further possibility is the analysis of all available references, which is much more complex. Therein all n references from a database (e.g. RefSeq) are extracted and compared with the newly sequenced bacterial genomes k. After this, matrices (% of mapped reads, % of covered genome) are applied to estimate which reference is best suited to all new bacteria. However, n×k complete alignments are carried out. Having a big number of references, though, stable results can be obtained, as is the case for Klebsiella, particularly Klebsiella pneumoniae and/or Klebsiella oxytoca.

According to certain embodiments, the genomes of Klebsiella species, particularly Klebsiella pneumoniae and/or Klebsiella oxytoca, are referenced to one reference genome. However, it is not excluded that for other microorganisms more than one reference genome is used. In the present methods, a reference genome of Klebsiella, particularly Klebsiella pneumoniae, is NC_009648, as annotated at the NCBI, and a reference genome of Klebsiella, particularly Klebsiella oxytoca, is NC_016612, as annotated at the NCBI, according to certain embodiments. The reference genomes are attached to this application as sequence listing.

One reference sequence was obtained from Klebsiella, particularly Klebsiella pneumoniae, strain NC_009648 (http://www.genome.jp/dbget-bin/www_bget?refseq+NC_009648) LOCUS NC_009648 5315120 bp DNA circular CON 7 Feb. 2015 DEFINITION Klebsiella pneumoniae subsp. pneumoniae MGH 78578, complete sequence.

| | |
|---|---|
| ACCESSION | NC_009648 |
| VERSION | NC_009648.1 GI: 152968582 |
| DBLINK | BioProject: PRJNA224116 |
| | BioSample: SAMN02603941 |
| | Assembly: GCF_000016305.1 |
| KEYWORDS | RefSeq. |
| SOURCE | Klebsiella pneumoniae subsp. pneumoniae MGH 78578 |
| ORGANISM | Klebsiella pneumoniae subsp. pneumoniae MGH 78578 |
| | Bacteria; Proteobacteria; Gammaproteobacteria; |
| | Enterobacteriales; Enterobacteriaceae; Klebsiella. |
| REFERENCE | 1 (bases 1 to 5315120) |
| AUTHORS | McClelland, M., Sanderson, E. K., Spieth, J., Clifton, |
| | W. S., Latreille, P., Sabo, A., Pepin, K., Bhonagiri, V., |
| | Porwollik, S., Ali, J. and Wilson, R. K. |
| CONSRTM | The Klebsiella pneumonia Genome Sequencing Project |
| TITLE | Direct Submission |
| JOURNAL | Submitted (06-SEP-2006) Genetics, Genome Sequencing |
| | Center, 4444 Forest Park Parkway, St. Louis, MO 63108, |
| | USA |

Another reference sequence was obtained from Klebsiella, particularly Klebsiella oxytoca, strain NC_016612 (http://www.genome.jp/dbget-bin/www_bget?refseq+NC_016612) LOCUS NC_016612 5974109 bp DNA circular CON 7 Feb. 2015 DEFINITION Klebsiella oxytoca KCTC 1686, complete genome.

| | |
|---|---|
| ACCESSION | NC_016612 |
| VERSION | NC_016612.1 GI: 375256816 |
| DBLINK | BioProject: PRJNA224116 |
| | BioSample: SAMN02603580 |
| | Assembly: GCF_000240325.1 |
| KEYWORDS | RefSeq. |
| SOURCE | Klebsiella oxytoca KCTC 1686 |
| ORGANISM | Klebsiella oxytoca KCTC 1686 |
| | Bacteria; Proteobacteria; Gammaproteobacteria; |
| | Enterobacteriales; Enterobacteriaceae; Klebsiella. |
| REFERENCE | 1 (bases 1 to 5974109) |
| AUTHORS | Shin, S. H., Kim, S., Kim, J. Y., Lee, S., Um, Y., |
| | Oh, M. K., Kim, Y. R., Lee, J. and Yang, K. S. |
| TITLE | Complete genome sequence of Klebsiella oxytoca |
| | KCTC 1686, used in production of 2,3-butanediol |
| JOURNAL | J. Bacteriol. 194 (9), 2371-2372 (2012) |
| PUBMED | 22493189 |
| REFERENCE | 2 (bases 1 to 5974109) |
| AUTHORS | Shin, S. H., Kim, S., Kim, J. Y., Yang, K.-S. and |
| | Seo, J.-S. |
| TITLE | Direct Submission |
| JOURNAL | Submitted (21-DEC-2011) Life Science Institute, |
| | Macrogen Inc., 10F, World Meridian Center, 60-24, |
| | Gasan-dong, Kumchun-gu, Seoul 153-781, Republic |
| | of Korea |

Alternatively or in addition, the gene sequence of the first data set can be assembled, at least in part, with known methods, e.g. by de-novo assembly or mapping assembly. The sequence assembly is not particularly limited, and any known genome assembler can be used, e.g. based on Sanger, 454, Solexa, Illumina, SOLid technologies, etc., as well as hybrids/mixtures thereof.

According to certain embodiments, the data of nucleic acids of different origin than the microorganism of interest, e.g. Klebsiella species, particularly Klebsiella pneumoniae and/or Klebsiella oxytoca, can be removed after the nucleic acids of interest are identified, e.g. by filtering the data out. Such data can e.g. include nucleic acids of the patient, e.g. the vertebrate, e.g. human, and/or other microorganisms, etc. This can be done by e.g. computational subtraction, as developed by Meyerson et al. 2002. For this, also aligning to the genome of the vertebrate, etc., is possible. For aligning, several alignment-tools are available. This way the original data amount from the sample can be drastically reduced.

Also after such removal of “excess” data, fingerprinting and/or aligning, and/or assembly, etc. can be carried out, as described above, forming a third data set of aligned and/or assembled genes for a Klebsiella species, particularly Klebsiella pneumoniae and/or Klebsiella oxytoca.

Using these techniques, genes with mutations of the microorganism of interest, e.g. Klebsiella species, particularly Klebsiella pneumoniae and/or Klebsiella oxytoca, can be obtained for various species.

When testing these same species for antimicrobial drug, e.g. antibiotic, susceptibility of a number of antimicrobial drugs, e.g. antibiotics, e.g. using standard culturing methods on dishes with antimicrobial drug, e.g. antibiotic, intake, as e.g. described below, the results of these antimicrobial drug, e.g. antibiotic, susceptibility tests can then be cross-referenced/correlated with the mutations in the genome of the respective microorganism, e.g. Klebsiella, particularly Klebsiella pneumoniae and/or Klebsiella oxytoca. Using several, e.g. 50 or more than 50, 100 or more than 100, 200 or more than 200, 250 or more than 250, 300 or more than 300, 350 or more than 350, 1000 or more than 1000, 1100 or more than 1100, different species of a microorganism, e.g. different Klebsiella species, particularly Klebsiella pneumoniae and/or Klebsiella oxytoca, statistical analysis can be carried out on the obtained cross-referenced data between mutations and antimicrobial drug, e.g. antibiotic, susceptibility for these number of species, using known methods.

Regarding culturing methods, samples can be e.g. cultured overnight. On the next day individual colonies can be used for identification of organisms, either by culturing or using mass spectroscopy. Based on the identity of organisms new plates containing increasing concentration of antibiotics used for the treatment of these organisms are inoculated and grown for additional 12-24 hours. The lowest drug concentration which inhibits growth (minimal inhibitory concentration—MIC) can be used to determine susceptibility/resistance for tested antibiotics.

Correlation of the nucleic acid/gene mutations with antimicrobial drug, e.g. antibiotic, resistance can be carried out in a usual way and is not particularly limited. For example, resistances can be correlated to certain genes or certain mutations, e.g. SNPs, in genes. After correlation, statistical analysis can be carried out.

In addition, statistical analysis of the correlation of the gene mutations with antimicrobial drug, e.g. antibiotic, resistance is not particularly limited and can be carried out, depending on e.g. the amount of data, in different ways, for example using analysis of variance (ANOVA) or Student's t-test, for example with a sample size n of 50, 100, 200, 250, 300, 350, 1000 or 1100, and a level of significance (α-error-level) of e.g. 0.05 or smaller, e.g. 0.05, preferably 0.01 or smaller. A statistical value can be obtained for each gene and/or each position in the genome as well as for all antibiotics tested, a group of antibiotics or a single antibiotic.

The obtained p-values can also be adapted for statistical errors, if needed.

For statistically sound results a multitude of individuals should be sampled, with n=50, 100, 200, 250, 300, 350, 1000 or 1100, and a level of significance (α-error-level) of e.g. 0.05 or smaller, e.g. 0.05, preferably 0.01 or smaller. According to certain embodiments, particularly significant results can be obtained for n=200, 250, 300, 350, 1000 or 1100.

For statistically sound results a multitude of individuals should be sampled, with n=50 or more, 100 or more, 200 or more, 250 or more, 300 or more, 350 or more, 1000 or more or 1100 or more, and a level of significance (α-error-level) of e.g. 0.05 or smaller, e.g. 0.05, preferably 0.01 or smaller. According to certain embodiments, particularly significant results can be obtained for n=200 or more, 250 or more, 300 or more, 350 or more, 1000 or more or 1100 or more.

After the above procedure has been carried out for more than 1100, e.g. 1176, and/or more than 350, e.g. 400, individual species of Klebsiella, particularly Klebsiella pneumoniae and/or Klebsiella oxytoca, respectively, the data disclosed in Tables 1a and 1b and 2a and 2b were obtained for the statistically best correlations between gene mutations and antimicrobial drug, e.g. antibiotic, resistances. Thus, mutations in these genes were proven as valid markers for antimicrobial drug, e.g. antibiotic, resistance.

According to a further aspect, the present invention relates in a second aspect to a method of selecting a treatment of a patient suffering from an infection with a potentially resistant Klebsiella, particularly Klebsiella pneumoniae and/or Klebsiella oxytoca, strain, e.g. from an antimicrobial drug, e.g. antibiotic, resistant Klebsiella, particularly Klebsiella pneumoniae and/or Klebsiella oxytoca, infection, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least two genes from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, wherein the presence of said at least two mutations is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection.

According to certain embodiments, the method of the second aspect relates to a method of selecting a treatment of a patient suffering from an infection with a potentially resistant Klebsiella, particularly Klebsiella pneumoniae, strain, e.g. from an antimicrobial drug, e.g. antibiotic, resistant Klebsiella, particularly Klebsiella pneumoniae, infection, comprising the steps of:

a) obtaining or providing a sample containing or suspected of containing at least one Klebsiella species, particularly Klebsiella pneumoniae, from the patient;
b) determining the presence of at least one mutation in at least two genes from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, wherein the presence of said at least two mutations is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae, infection.

According to certain embodiments, the method of the second aspect relates to a method of selecting a treatment of a patient suffering from an infection with a potentially resistant Klebsiella, particularly Klebsiella oxytoca, strain, e.g. from an antimicrobial drug, e.g. antibiotic, resistant Klebsiella, particularly Klebsiella oxytoca, infection, comprising the steps of:

a) obtaining or providing a sample containing or suspected of containing at least one Klebsiella species, particularly Klebsiella oxytoca, from the patient;
b) determining the presence of at least one mutation in at least two genes from the group of genes consisting of KOX_26125, KOX_13365, KOX_16735, KOX_25695, KOX_12270, and KOX_15055, wherein the presence of said at least two mutations is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella oxytoca, infection.

In this method, the steps a) of obtaining or providing a sample and b) of determining the presence of at least one mutation are as in the method of the first aspect.

The identification of the at least one or more antimicrobial, e.g. antibiotic, drug in step c) is then based on the results obtained in step b) and corresponds to the antimicrobial, e.g. antibiotic, drug(s) that correlate(s) with the mutations. Once these antimicrobial drugs, e.g. antibiotics, are ruled out, the remaining antimicrobial drugs, e.g. antibiotic drugs/antibiotics, can be selected in step d) as being suitable for treatment.

In the description, references to the first and second aspect also apply to the 14^{th}, 15^{th}, 16^{th} and 17^{th} aspect, referring to the same genes, unless clear from the context that they don't apply.

According to certain embodiments in the method of the first or second aspect, the Klebsiella species is Klebsiella pneumoniae and at least a mutation in parC, particularly in position 3763210 with regard to reference genome NC_009648 as annotated at the NCBI, is determined. For such mutation, a particularly relevant correlation with antimicrobial drug, e.g. antibiotic, resistance could be determined. In particular, the mutation in position 3763210 with regard to reference genome NC_009648 as annotated at the NCBI results in a non-synonymous substitution, particularly a codon change aGc/aTc.

According to certain embodiments in the method of the first or second aspect, the Klebsiella species is Klebsiella oxytoca and at least a mutation in KOX_26125, particularly in position 5645611, with regard to reference genome NC_016612 as annotated at the NCBI, is determined. For such mutations, a particularly relevant correlation with antimicrobial drug, e.g. antibiotic, resistance could be determined. In particular, the mutation in positions 5645611 with regard to reference genome NC_016612 as annotated at the NCBI results in a non-synonymous substitution, particularly a codon change aCt/aTt.

According to certain embodiments, the antimicrobial drug, e.g. antibiotic, in the method of the first or second aspect, as well as in the other methods of the invention, is at least one selected from the group of β-lactams, β-lactam inhibitors, quinolines and derivatives thereof, aminoglycosides, polyketides, respectively tetracyclines, and folate synthesis inhibitors.

In the methods of the invention the resistance of Klebsiella, particularly Klebsiella pneumoniae and/or Klebsiella oxytoca, to one or more antimicrobial, e.g. antibiotic, drugs can be determined according to certain embodiments.

According to certain embodiments of the first and/or second aspect of the invention the antimicrobial, e.g. antibiotic, drug is selected from lactam antibiotics and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195, and/or KOX_26125, KOX_13365, KOX_16735, KOX_25695, KOX_12270, and/or KOX_15055.

According to certain embodiments of the first and/or second aspect of the invention resistance to Klebsiella pneumoniae is determined, the antimicrobial, e.g. antibiotic, drug is selected from lactam antibiotics and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195.

According to certain embodiments of the first and/or second aspect of the invention resistance to Klebsiella oxytoca is determined, the antimicrobial, e.g. antibiotic, drug is selected from lactam antibiotics and the presence of a mutation in the following genes is determined: KOX_26125, KOX_13365, KOX_16735, KOX_25695, KOX_12270, and/or KOX_15055

According to certain embodiments of the first and/or second aspect of the invention the antimicrobial, e.g. antibiotic, drug is selected from quinolone antibiotics, particularly fluoroquinolone antibiotics, and/or polyketide antibiotics, particularly tetracycline antibiotics, and/or benzene derived/sulfonamide antibiotics, and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195, and/or KOX_26125.

According to certain embodiments of the first and/or second aspect of the invention resistance to Klebsiella pneumoniae is determined, the antimicrobial, e.g. antibiotic, drug is selected from quinolone antibiotics, particularly fluoroquinolone antibiotics, and/or polyketide antibiotics, particularly tetracycline antibiotics, and/or benzene derived/sulfonamide antibiotics, and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195.

According to certain embodiments of the first and/or second aspect of the invention resistance to Klebsiella oxytoca is determined, the antimicrobial, e.g. antibiotic, drug is selected from quinolone antibiotics, particularly fluoroquinolone antibiotics, and/or polyketide antibiotics, particularly tetracycline antibiotics, and/or benzene derived/sulfonamide antibiotics, and the presence of a mutation in the following genes is determined: KOX_26125.

According to certain embodiments of the first and/or second aspect of the invention the antimicrobial, e.g. antibiotic, drug is selected from aminoglycoside antibiotics and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195.

According to certain embodiments of the first and/or second aspect of the invention resistance to Klebsiella pneumoniae is determined, the antimicrobial, e.g. antibiotic, drug is selected from aminoglycoside antibiotics and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195.

According to certain embodiments, the antimicrobial drug is an antibiotic/antibiotic drug.

According to certain embodiments of the first and/or second aspect of the invention, determining the nucleic acid sequence information or the presence of a mutation comprises determining the presence of a single nucleotide at a single position in a gene. Thus the invention comprises methods wherein the presence of a single nucleotide polymorphism or mutation at a single nucleotide position is detected.

According to certain embodiments, the antibiotic drug in the methods of the present invention is selected from the group consisting of Amoxicillin/K Clavulanate (AUG), Ampicillin (AM), Aztreonam (AZT), Cefazolin (CFZ), Cefepime (CPE), Cefotaxime (CFT), Ceftazidime (CAZ), Ceftriaxone (CAX), Cefuroxime (CRM), Cephalotin (CF), Ciprofloxacin (CP), Ertapenem (ETP), Gentamicin (GM), Imipenem (IMP), Levofloxacin (LVX), Meropenem (MER), Piperacillin/Tazobactam (P/T), Ampicillin/Sulbactam (A/S), Tetracycline (TE), Tobramycin (TO), and Trimethoprim/Sulfamethoxazole (T/S).

The inventors have surprisingly found that mutations in certain genes are indicative not only for a resistance to one single antimicrobial, e.g. antibiotic, drug, but to groups containing several drugs.

According to certain embodiments of the first and/or second aspect of the invention, resistance to Klebsiella pneumoniae is determined, the gene is from Table 1a and/or Table 2a, particularly Table 2a, the antibiotic drug is selected from lactam antibiotics, and a mutation in at least one of the following genes is detected with regard to reference genome NC_009648: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195.

According to certain embodiments of the first and/or second aspect of the invention, resistance to Klebsiella oxytoca is determined, the gene is from Table 1b and/or Table 2b, particularly Table 2b, the antibiotic drug is selected from lactam antibiotics, and a mutation in at least one of the following genes is detected with regard to reference genome NC_016612: KOX_26125, KOX_13365, KOX_16735, KOX_25695, KOX_12270, and/or KOX_15055.

According to certain embodiments of the first and/or second aspect of the invention, resistance to Klebsiella pneumoniae is determined, the gene is from Table 1a and/or Table 2a, particularly Table 2a, the antibiotic drug is selected from quinolone antibiotics, particularly fluoroquinolone antibiotics, and/or polyketide antibiotics, particularly tetracycline antibiotics, and/or benzene derived/sulfonamide antibiotics, and a mutation in at least one of the following genes is detected with regard to reference genome NC_009648: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195.

According to certain embodiments of the first and/or second aspect of the invention, resistance to Klebsiella oxytoca is determined, the gene is from Table 1b and/or Table 2b, particularly Table 2b, the antibiotic drug is selected from quinolone antibiotics, particularly fluoroquinolone antibiotics, and/or polyketide antibiotics, particularly tetracycline antibiotics, and/or benzene derived/sulfonamide antibiotics, and a mutation in at least one of the following genes is detected with regard to reference genome NC_016612: KOX_26125.

According to certain embodiments of the first and/or second aspect of the invention, resistance to Klebsiella pneumoniae is determined, the gene is from Table 1a and/or Table 2a, particularly Table 2a, the antibiotic drug is selected from aminoglycoside antibiotics, and a mutation in at least one of the following genes is detected with regard to reference genome NC_009648: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195.

For specific antimicrobial drugs, e.g. antibiotics, specific positions in the above genes can be determined where a high statistical significance is observed. The inventors found that, apart from the above genes indicative of a resistance against antibiotics, also single nucleotide polymorphisms (=SNP's) may have a high significance for the presence of a resistance against defined antibiotic drugs. The analysis of these polymorphisms on a nucleotide level may further improve and accelerate the determination of a drug resistance to antimicrobial drugs, e.g. antibiotics, in Klebsiella, particularly Klebsiella pneumoniae and/or Klebsiella oxytoca.

According to certain embodiments of the first and/or second aspect of the invention, resistance to Klebsiella pneumoniae is determined, the gene is from Table 1a and/or Table 2a, particularly Table 2a, the antibiotic drug is selected from lactam antibiotics, and a mutation in at least one of the following nucleotide positions is detected with regard to reference genome NC_009648: 3763210, 1784305, 1784302, 2905411, 2673906, 2773232, 140517, 809148, 1364586, 2150691, 2159024, 2317024, 2325877, 2331649, 2347930, 2355785, 2365629, 2375692, 2402871, 2459360, 2517274, 2521829, 2532012, 2547536, 2629283, 2658497, 2703286, 2774521, 2812941, 2831238, 2875745, 2878878, 2920245, 2379716, 2218319, 2504346, 2505230, 2506816, 2641631, 2646728, 1704769, 2524562, 2772839, 2627362, 2124017, 2174754, 2275805, 2662814, 2784148, 1933723, 4595554.

According to certain embodiments of the first and/or second aspect of the invention, resistance to Klebsiella oxytoca is determined, the gene is from Table 1b and/or Table 2b, particularly Table 2b, the antibiotic drug is selected from lactam antibiotics, and a mutation in at least one of the following nucleotide positions is detected with regard to reference genome NC_016612: 5645611, 2887469, 2887473, 3631990, 5544665, 5544668, 2652345, 3260573.

According to certain embodiments of the first and/or second aspect of the invention, resistance to Klebsiella pneumoniae is determined, the gene is from Table 1a and/or Table 2a, particularly Table 2a, the antibiotic drug is selected from quinolone antibiotics, particularly fluoroquinolone antibiotics, and/or polyketide antibiotics, particularly tetracycline antibiotics, and/or benzene derived/sulfonamide antibiotics, and a mutation in at least one of the following nucleotide positions is detected with regard to reference genome NC_009648: 3763210, 1784305, 1784302, 2905411, 2673906, 2773232, 140517, 809148, 1364586, 2150691, 2159024, 2317024, 2325877, 2331649, 2347930, 2355785, 2365629, 2375692, 2402871, 2459360, 2517274, 2521829, 2532012, 2547536, 2629283, 2658497, 2703286, 2774521, 2812941, 2831238, 2875745, 2878878, 2920245, 2379716, 2218319, 2504346, 2505230, 2506816, 2641631, 2646728, 1704769, 2524562, 2772839, 2627362, 2124017, 2174754, 2275805, 2662814, 2784148, 1933723, 4595554.

According to certain embodiments of the first and/or second aspect of the invention, resistance to Klebsiella oxytoca is determined, the gene is from Table 1b and/or Table 2b, particularly Table 2b, the antibiotic drug is selected from quinolone antibiotics, particularly fluoroquinolone antibiotics, and/or polyketide antibiotics, particularly tetracycline antibiotics, and/or benzene derived/sulfonamide antibiotics, and a mutation in at least one of the following nucleotide positions is detected with regard to reference genome NC_016612: 5645611.

According to certain embodiments of the first and/or second aspect of the invention, resistance to Klebsiella pneumoniae is determined, the gene is from Table 1a and/or Table 2a, particularly Table 2a, the antibiotic drug is selected from aminoglycoside antibiotics, and a mutation in at least one of the following nucleotide positions is detected with regard to reference genome NC_009648: 3763210, 1784305, 1784302, 2905411, 2673906, 2773232, 140517, 809148, 1364586, 2150691, 2159024, 2317024, 2325877, 2331649, 2347930, 2355785, 2365629, 2375692, 2402871, 2459360, 2517274, 2521829, 2532012, 2547536, 2629283, 2658497, 2703286, 2774521, 2812941, 2831238, 2875745, 2878878, 2920245, 2379716, 2218319, 2504346, 2505230, 2506816, 2641631, 2646728, 1704769, 2524562, 2772839, 2627362, 2124017, 2174754, 2275805, 2662814, 2784148, 1933723, 4595554.

According to certain embodiments of the first and/or second aspect of the invention, resistance to Klebsiella pneumoniae is determined, the antibiotic drug is at least one of CF, CFT, IMP, CFZ, CRM, ETP, CAX, AZT, P/T, CPE, AM, A/S, CAZ, MER, AUG, CP, LVX, GM, TO, TE, and T/S and a mutation in at least one of the following nucleotide positions is detected with regard to reference genome NC_009648: 3763210, 1784305, 1784302, 2905411, 2673906, 2773232, 140517, 809148, 1364586, 2150691, 2159024, 2317024, 2325877, 2331649, 2347930, 2355785, 2365629, 2375692, 2402871, 2459360, 2517274, 2521829, 2532012, 2547536, 2629283, 2658497, 2703286, 2774521, 2812941, 2831238, 2875745, 2878878, 2920245, 2379716, 2218319, 2504346, 2505230, 2506816, 2641631, 2646728, 1704769, 2524562, 2772839, 2627362, 2124017, 2174754, 2275805, 2662814, 2784148, 1933723, 4595554.

According to certain embodiments of the first and/or second aspect of the invention, resistance to Klebsiella oxytoca is determined, the antibiotic drug is at least one of CF, CFZ, CRM, AZT, AM, and A/S and a mutation in at least one of the following nucleotide positions is detected with regard to reference genome NC_016612: 5645611, 2887469, 2887473, 3631990, 5544665, 5544668, 2652345, 3260573.

According to certain embodiments of the first and/or second aspect of the invention, resistance to Klebsiella oxytoca is determined, the antibiotic drug is at least one of CFT, CAX, P/T, CPE, CAZ, AUG, CP, LVX, TE, and T/S and a mutation in at least one of the following nucleotide positions is detected with regard to reference genome NC_016612: 5645611.

Although the genes and gene positions with regard to the antibiotic classes and the specific antibiotics have been described above separately for the two reference genomes for the sake of brevity, also the results from the different list for the same antibiotic classes and/or the specific antibiotics can be combined according to certain embodiments of the invention.

According to certain embodiments of the first and/or second aspect of the invention, the resistance of a bacterial microorganism belonging to the species Klebsiella, particularly Klebsiella pneumoniae and/or Klebsiella oxytoca, against 1, 2, 3, 4, 5, 6, 7, 8, 9, 10, 11, 12, 13, 14, 15 or 16, 17, 18, 19, 20 or 21 antibiotic drugs is determined.

According to certain embodiments of the first and/or second aspect of the invention, a detected mutation is a mutation leading to an altered amino acid sequence in a polypeptide derived from a respective gene in which the detected mutation is located. According to this aspect, the detected mutation thus leads to a truncated version of the polypeptide (wherein a new stop codon is created by the mutation) or a mutated version of the polypeptide having an amino acid exchange at the respective position.

According to certain embodiments of the first and/or second aspect of the invention, determining the nucleic acid sequence information or the presence of a mutation comprises determining a partial sequence or an entire sequence of the at least two genes.

According to certain embodiments of the first and/or second aspect of the invention, determining the nucleic acid sequence information or the presence of a mutation comprises determining a partial or entire sequence of the genome of the Klebsiella species, particularly Klebsiella pneumoniae and/or Klebsiella oxytoca, wherein said partial or entire sequence of the genome comprises at least a partial sequence of said at least two genes.

According to certain embodiments of the first and/or second aspect of the invention, determining the nucleic acid sequence information or the presence of a mutation comprises using a next generation sequencing or high throughput sequencing method. According to preferred embodiments of the first and/or second aspect of the invention, a partial or entire genome sequence of the bacterial organism of Klebsiella species, particularly Klebsiella pneumoniae and/or Klebsiella oxytoca, is determined by using a next generation sequencing or high throughput sequencing method.

In a further, third aspect, the present invention relates to a method of determining an antimicrobial drug, e.g. antibiotic, resistance profile for bacterial microorganisms of Klebsiella species, particularly Klebsiella pneumoniae and/or Klebsiella oxytoca, comprising:

obtaining or providing a first data set of gene sequences of a plurality of clinical isolates of Klebsiella species, particularly Klebsiella pneumoniae and/or Klebsiella oxytoca;
providing a second data set of antimicrobial drug, e.g. antibiotic, resistance of the plurality of clinical isolates of Klebsiella species, particularly Klebsiella pneumoniae and/or Klebsiella oxytoca;
aligning the gene sequences of the first data set to at least one, preferably one, reference genome of Klebsiella, particularly Klebsiella pneumoniae and/or Klebsiella oxytoca, and/or assembling the gene sequence of the first data set, at least in part;
analyzing the gene sequences of the first data set for genetic variants to obtain a third data set of genetic variants; correlating the third data set with the second data set and statistically analyzing the correlation; and
determining the genetic sites in the genome of Klebsiella, particularly Klebsiella pneumoniae and/or Klebsiella oxytoca, associated with antimicrobial drug, e.g. antibiotic, resistance.

The different steps can be carried out as described with regard to the method of the first aspect of the present invention.

When referring to the second data set, wherein the second data set e.g. comprises, respectively is, a set of antimicrobial drug, e.g. antibiotic, resistances of a plurality of clinical isolates, this can, within the scope of the invention, also refer to a self-learning data base that, whenever a new sample is analyzed, can take this sample into the second data set and thus expand its data base. The second data set thus does not have to be static and can be expanded, either by external input or by incorporating new data due to self-learning. This is, however, not restricted to the third aspect of the invention, but applies to other aspects of the invention that refer to a second data set, which does not necessarily have to refer to antimicrobial drug resistance. The same applies, where applicable, to the first data set, e.g. in the third aspect.

According to certain embodiments, statistical analysis in the present methods is carried out using Fisher's test with p<10⁻⁶, preferably p<10⁻⁹, particularly p<10⁻¹⁰, particularly p<10⁻¹¹.

The method of the third aspect of the present invention, as well as related methods, e.g. according to the 7^{th} and 10^{th} aspect, can, according to certain embodiments, comprise correlating different genetic sites to each other. This way even higher statistical significance can be achieved.

According to certain embodiments of the method of the third aspect and related methods—as above, the second data set is provided by culturing the clinical isolates of Klebsiella species, particularly Klebsiella pneumoniae and/or Klebsiella oxytoca, on agar plates provided with antimicrobial drugs, e.g. antibiotics, at different concentrations and the second data is obtained by taking the minimal concentration of the plates that inhibits growth of the respective Klebsiella species, particularly Klebsiella pneumoniae and/or Klebsiella oxytoca.

According to certain embodiments of the method of the third aspect and related methods, the antibiotic is at least one selected from the group of β-lactams, β-lactam inhibitors, quinolines and derivatives thereof, aminoglycosides, tetracyclines, and folate synthesis inhibitors, preferably Amoxicillin/K Clavulanate, Ampicillin, Aztreonam, Cefazolin, Cefepime, Cefotaxime, Ceftazidime, Ceftriaxone, Cefuroxime, Cephalothin, Ciprofloxacin, Ertapenem, Gentamicin, Imipenem, Levofloxacin, Meropenem, Piperacillin/Tazobactam, Ampicillin/Sulbactam, Tetracycline, Tobramycin, and Trimethoprim/Sulfamethoxazole.

According to certain embodiments of the method of the third aspect and related methods, the gene sequences in the third data set are comprised in at least one gene from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, or from the genes listed in Table 5a and/or Table 5b.

According to certain embodiments of the method of the third aspect and related methods, an antimicrobial drug, e.g. antibiotic, resistance profile for bacterial microorganisms of Klebsiella pneumoniae is determined and the gene sequences in the third data set are comprised in at least one gene from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, or from the genes listed in Table 5a.

According to certain embodiments of the method of the third aspect and related methods, an antimicrobial drug, e.g. antibiotic, resistance profile for bacterial microorganisms of Klebsiella oxytoca is determined and the gene sequences in the third data set are comprised in at least one gene from the group of genes consisting of KOX_26125, KOX_13365, KOX_16735, KOX_25695, KOX_12270, and KOX_15055, or from the genes listed in Table 5b.

According to certain embodiments of the method of the third aspect and related methods, the genetic sites in the genome of Klebsiella associated with antimicrobial drug, e.g. antibiotic, resistance are at least comprised in one gene from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055.

According to certain embodiments of the method of the third aspect and related methods, an antimicrobial drug, e.g. antibiotic, resistance profile for bacterial microorganisms of Klebsiella pneumoniae is determined and the genetic sites in the genome of Klebsiella associated with antimicrobial drug, e.g. antibiotic, resistance are at least comprised in one gene from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195.

According to certain embodiments of the method of the third aspect and related methods, an antimicrobial drug, e.g. antibiotic, resistance profile for bacterial microorganisms of Klebsiella oxytoca is determined and the genetic sites in the genome of Klebsiella associated with antimicrobial drug, e.g. antibiotic, resistance are at least comprised in one gene from the group of genes consisting of KOX_26125, KOX_13365, KOX_16735, KOX_25695, KOX_12270, and KOX_15055.

According to certain embodiments of the method of the third aspect and related methods, the genetic variant has a point mutation, an insertion and or deletion of up to four bases, and/or a frameshift mutation, particularly a non-synonymous coding in YP 001337063.1 in case of Klebsiella pneumoniae and/or a non-synonymous coding in YP 005021173.1 in case of Klebsiella oxytoca.

A fourth aspect of the present invention relates to a method of determining an antimicrobial drug, e.g. antibiotic, resistance profile for a bacterial microorganism belonging to the species Klebsiella, particularly Klebsiella pneumoniae and/or Klebsiella oxytoca, comprising the steps of

a) obtaining or providing a sample containing or suspected of containing the bacterial microorganism;
b) determining the presence of a mutation in at least one gene of the bacterial microorganism as determined by the method of the third aspect of the invention;
wherein the presence of a mutation is indicative of a resistance to an antimicrobial drug, e.g. antibiotic, drug.

Steps a) and b) can herein be carried out as described with regard to the first aspect, as well as for the following aspects of the invention.

With this method, any mutations in the genome of Klebsiella species, particularly Klebsiella pneumoniae and/or Klebsiella oxytoca, correlated with antimicrobial drug, e.g. antibiotic, resistance can be determined and a thorough antimicrobial drug, e.g. antibiotic, resistance profile can be established.

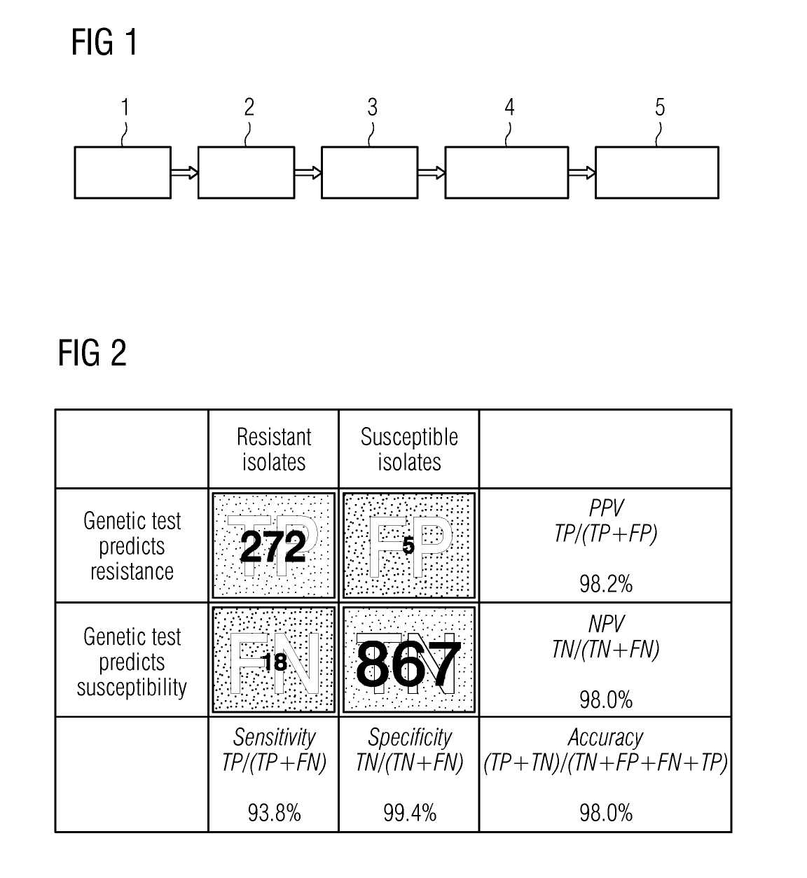

A simple read out concept for a diagnostic test as described in this aspect is shown schematically in FIG. 1.

According to FIG. 1, a sample 1, e.g. blood from a patient, is used for molecular testing 2, e.g. using next generation sequencing (NGS), and then a molecular fingerprint 3 is taken, e.g. in case of NGS a sequence of selected genomic/plasmid regions or the whole genome is assembled. This is then compared to a reference library 4, i.e. selected sequences or the whole sequence are/is compared to one or more reference sequences, and mutations (SNPs, sequence-gene additions/deletions, etc.) are correlated with susceptibility/resistance profile of reference strains in the reference library. The reference library 4 herein contains many genomes and is different from a reference genome. Then the result 5 is reported comprising ID (pathogen identification), i.e. a list of all (pathogenic) species identified in the sample, and AST (antimicrobial susceptibility testing), i.e. a list including a susceptibility/resistance profile for all species listed.

A fifth aspect of the present invention relates to a diagnostic method of determining an infection of a patient with Klebsiella species, particularly Klebsiella pneumoniae and/or Klebsiella oxytoca, potentially resistant to antimicrobial drug treatment, which also can be described as method of determining an antimicrobial drug, e.g. antibiotic, resistant Klebsiella, particularly Klebsiella pneumoniae and/or Klebsiella oxytoca, infection in a patient, comprising the steps of:

a) obtaining or providing a sample containing or suspected of containing a bacterial microorganism belonging to the species Klebsiella, particularly Klebsiella pneumoniae and/or Klebsiella oxytoca, from the patient;
b) determining the presence of at least one mutation in at least one gene of the bacterial microorganism belonging to the species Klebsiella, particularly Klebsiella pneumoniae and/or Klebsiella oxytoca, as determined by the method of the third aspect of the present invention, wherein the presence of said at least one mutation is indicative of an antimicrobial drug, e.g. antibiotic, resistant Klebsiella, particularly Klebsiella pneumoniae and/or Klebsiella oxytoca, infection in said patient.

Again, steps a) and b) can herein be carried out as described with regard to the first aspect of the present invention.

According to this aspect, a Klebsiella, particularly Klebsiella pneumoniae and/or Klebsiella oxytoca, infection in a patient can be determined using sequencing methods as well as a resistance to antimicrobial drugs, e.g. antibiotics, of the Klebsiella species, particularly Klebsiella pneumoniae and/or Klebsiella oxytoca, be determined in a short amount of time compared to the conventional methods.

In a sixth aspect the present invention relates to a method of selecting a treatment of a patient suffering from an infection with a potentially resistant Klebsiella, particularly Klebsiella pneumoniae and/or Klebsiella oxytoca, strain, e.g. an antimicrobial drug, e.g. antibiotic, resistant Klebsiella, particularly Klebsiella pneumoniae and/or Klebsiella oxytoca, infection, comprising the steps of:

a) obtaining or providing a sample containing or suspected of containing a bacterial microorganism belonging to the species Klebsiella, particularly Klebsiella pneumoniae and/or Klebsiella oxytoca, from the patient;
b) determining the presence of at least one mutation in at least one gene of the bacterial microorganism belonging to the species Klebsiella, particularly Klebsiella pneumoniae and/or Klebsiella oxytoca, as determined by the method of the third aspect of the invention, wherein the presence of said at least one mutation is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection.

This method can be carried out similarly to the second aspect of the invention and enables a fast was to select a suitable treatment with antibiotics for any infection with an unknown Klebsiella species, particularly Klebsiella pneumoniae and/or Klebsiella oxytoca.

A seventh aspect of the present invention relates to a method of acquiring, respectively determining, an antimicrobial drug, e.g. antibiotic, resistance profile for a bacterial microorganisms of Klebsiella species, particularly Klebsiella pneumoniae and/or Klebsiella oxytoca, comprising:

obtaining or providing a first data set of gene sequences of a clinical isolate of Klebsiella species, particularly Klebsiella pneumoniae and/or Klebsiella oxytoca;
providing a second data set of antimicrobial drug, e.g. antibiotic, resistance of a plurality of clinical isolates of Klebsiella species, particularly Klebsiella pneumoniae and/or Klebsiella oxytoca;
aligning the gene sequences of the first data set to at least one, preferably one, reference genome of Klebsiella, particularly Klebsiella pneumoniae and/or Klebsiella oxytoca, and/or assembling the gene sequence of the first data set, at least in part;
analyzing the gene sequences of the first data set for genetic variants to obtain a third data set of genetic variants of the first data set;
correlating the third data set with the second data set and statistically analyzing the correlation; and
determining the genetic sites in the genome of Klebsiella, particularly Klebsiella pneumoniae and/or Klebsiella oxytoca, of the first data set associated with antimicrobial drug, e.g. antibiotic, resistance.

With this method, antimicrobial drug, e.g. antibiotic, resistances in an unknown isolate of Klebsiella, particularly Klebsiella pneumoniae and/or Klebsiella oxytoca, can be determined.

According to certain embodiments, the reference genome of Klebsiella, particularly Klebsiella pneumoniae and/or Klebsiella oxytoca, is NC_009648 and/or NC_016612, as annotated at the NCBI. According to certain embodiments, the reference genome of Klebsiella pneumoniae is NC_009648 and the reference genome of Klebsiella oxytoca is NC_016612, as annotated at the NCBI. According to certain embodiments, statistical analysis in the present methods is carried out using Fisher's test with p<10⁻⁶, preferably p<10⁻⁹, particularly p<10⁻¹⁰, particularly p<10⁻¹¹. Also, according to certain embodiments, the method further comprises correlating different genetic sites to each other.

An eighth aspect of the present invention relates to a computer program product comprising computer executable instructions which, when executed, perform a method according to the third, fourth, fifth, sixth or seventh aspect of the present invention.

In certain embodiments the computer program product is one on which program commands or program codes of a computer program for executing said method are stored. According to certain embodiments the computer program product is a storage medium. The same applies to the computer program products of the aspects mentioned afterwards, i.e. the eleventh aspect of the present invention. As noted above, the computer program products of the present invention can be self-learning, e.g. with respect to the first and second data sets.

In order to obtain the best possible information from the highly complex genetic data and develop an optimum model for diagnostic and therapeutical uses as well as the methods of the present invention—which can be applied stably in clinical routine—a thorough in silico analysis can be necessary. The proposed principle is based on a combination of different approaches, e.g. alignment with at least one, preferably more reference genomes and/or assembly of the genome and correlation of mutations found in every sample, e.g. from each patient, with all references and drugs, e.g. antibiotics, and search for mutations which occur in several drug and several strains.

Using the above steps a list of mutations as well as of genes is generated. These can be stored in databases and statistical models can be derived from the databases. The statistical models can be based on at least one or more mutations in at least one or more genes. Statistical models that can be trained can be combined from mutations and genes. Examples of algorithms that can produce such models are association Rules, Support Vector Machines, Decision Trees, Decision Forests, Discriminant-Analysis, Cluster-Methods, and many more.

The goal of the training is to allow a reproducible, standardized application during routine procedures.

For this, for example, a genome or parts of the genome of a microorganism can be sequenced from a patient to be diagnosed. Afterwards, core characteristics can be derived from the sequence data which can be used to predict resistance.

These are the points in the database used for the final model, i.e. at least one mutation or at least one gene, but also combinations of mutations, etc.

The corresponding characteristics can be used as input for the statistical model and thus enable a prognosis for new patients. Not only the information regarding all resistances of all microorganisms, e.g. of Klebsiella species, particularly Klebsiella pneumoniae and/or Klebsiella oxytoca, against all drugs, e.g. antibiotics, can be integrated in a computer decision support tool, but also corresponding directives (e.g. EUCAST) so that only treatment proposals are made that are in line with the directives.

A ninth aspect of the present invention relates to the use of the computer program product according to the eighth aspect for acquiring an antimicrobial drug, e.g. antibiotic, resistance profile for bacterial microorganisms of Klebsiella species, particularly Klebsiella pneumoniae and/or Klebsiella oxytoca, or in a method of the third aspect of the invention.

In a tenth aspect a method of selecting a treatment of a patient having an infection with a bacterial microorganisms of Klebsiella species, particularly Klebsiella pneumoniae and/or Klebsiella oxytoca, comprising:

obtaining or providing a first data set comprising a gene sequence of at least one clinical isolate of Klebsiella, particularly Klebsiella pneumoniae and/or Klebsiella oxytoca, from the patient;
providing a second data set of antimicrobial drug, e.g. antibiotic, resistance of a plurality of clinical isolates of Klebsiella, particularly Klebsiella pneumoniae and/or Klebsiella oxytoca;
aligning the gene sequences of the first data set to at least one, preferably one, reference genome of Klebsiella, particularly Klebsiella pneumoniae and/or Klebsiella oxytoca, and/or assembling the gene sequence of the first data set, at least in part;
analyzing the gene sequences of the first data set for genetic variants to obtain a third data set of genetic variants of the first data set;
correlating the third data set with the second data set of antimicrobial drug, e.g. antibiotic, resistance of a plurality of clinical isolates of Klebsiella, particularly Klebsiella pneumoniae and/or Klebsiella oxytoca, and statistically analyzing the correlation;
determining the genetic sites in the genome of the clinical isolate of Klebsiella, particularly Klebsiella pneumoniae and/or Klebsiella oxytoca, of the first data set associated with antimicrobial drug, e.g. antibiotic, resistance; and selecting a treatment of the patient with one or more antimicrobial, e.g. antibiotic, drugs different from the ones identified in the determination of the genetic sites associated with antimicrobial drug, e.g. antibiotic, resistance is disclosed.

Again, the steps can be carried out as similar steps before. In this method, as well as similar ones, no aligning is necessary, as the unknown sample can be directly correlated, after the genome or genome sequences are produced, with the second data set and thus mutations and antimicrobial drug, e.g. antibiotic, resistances can be determined. The first data set can be assembled, for example, using known techniques.

According to certain embodiments, statistical analysis in the present method is carried out using Fisher's test with p<10⁻⁶, preferably p<10⁻⁹, particularly p<10⁻¹⁰, particularly p<10⁻¹¹. Also, according to certain embodiments, the method further comprises correlating different genetic sites to each other.

An eleventh aspect of the present invention is directed to a computer program product comprising computer executable instructions which, when executed, perform a method according to the tenth aspect.

According to a twelfth aspect of the present invention, a diagnostic method of determining an infection of a patient with Klebsiella species, particularly Klebsiella pneumoniae and/or Klebsiella oxytoca, potentially resistant to antimicrobial drug treatment, which can also be described as a method of determining an antimicrobial drug, e.g. antibiotic, resistant Klebsiella, particularly Klebsiella pneumoniae and/or Klebsiella oxytoca, infection of a patient is disclosed, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least two genes from the group of genes listed in Table 5a and/or Table 5b, wherein the presence of said at least two mutations is indicative of an antimicrobial drug, e.g. antibiotic, resistant Klebsiella, particularly Klebsiella pneumoniae and/or Klebsiella oxytoca, infection in said patient.

According to certain embodiments of the twelfth aspect, a diagnostic method of determining an infection of a patient with Klebsiella pneumoniae potentially resistant to antimicrobial drug treatment, which can also be described as a method of determining an antimicrobial drug, e.g. antibiotic, resistant Klebsiella pneumoniae infection of a patient is disclosed, comprising the steps of:

a) obtaining or providing a sample containing or suspected of containing at least one Klebsiella pneumoniae strain from the patient;
b) determining the presence of at least one mutation in at least two genes from the group of genes listed in Table 5a, wherein the presence of said at least two mutations is indicative of an antimicrobial drug, e.g. antibiotic, resistant Klebsiella pneumoniae infection in said patient.

According to certain embodiments of the twelfth aspect, a diagnostic method of determining an infection of a patient with Klebsiella oxytoca potentially resistant to antimicrobial drug treatment, which can also be described as a method of determining an antimicrobial drug, e.g. antibiotic, resistant Klebsiella oxytoca infection of a patient is disclosed, comprising the steps of:

a) obtaining or providing a sample containing or suspected of containing at least one Klebsiella oxytoca strain from the patient;
b) determining the presence of at least one mutation in at least two genes from the group of genes listed in Table 5b, wherein the presence of said at least two mutations is indicative of an antimicrobial drug, e.g. antibiotic, resistant Klebsiella oxytoca infection in said patient.

A thirteenth aspect of the invention discloses a method of selecting a treatment of a patient suffering from an antimicrobial drug, e.g. antibiotic, resistant Klebsiella, particularly Klebsiella pneumoniae and/or Klebsiella oxytoca infection, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least two genes from the group of genes listed in Table 5a and/or Table 5b, wherein the presence of said at least two mutations is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection.

According to certain embodiments the thirteenth aspect relates to a method of selecting a treatment of a patient suffering from an antimicrobial drug, e.g. antibiotic, resistant Klebsiella pneumoniae infection, comprising the steps of:

a) obtaining or providing a sample containing or suspected of containing at least one Klebsiella pneumoniae strain from the patient;
b) determining the presence of at least one mutation in at least two genes from the group of genes listed in Table 5a, wherein the presence of said at least two mutations is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella pneumoniae infection.

According to certain embodiments the thirteenth aspect relates to a method of selecting a treatment of a patient suffering from an antimicrobial drug, e.g. antibiotic, resistant Klebsiella oxytoca infection, comprising the steps of:

a) obtaining or providing a sample containing or suspected of containing at least one Klebsiella oxytoca strain from the patient;
b) determining the presence of at least one mutation in at least two genes from the group of genes listed in Table 5b, wherein the presence of said at least two mutations is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella oxytoca infection.

Again, the steps can be carried out as in similar methods before, e.g. as in the first and second aspect of the invention. In the twelfth and thirteenth aspect of the invention, all classes of antibiotics considered in the present method are covered.

Herein, the genes in Table 5a, particularly relating to Klebsiella pneumoniae, are the following:

parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, KPN_04195.

Herein, the genes in Table 5b, particularly relating to Klebsiella oxytoca, are the following:

KOX_26125, KOX_13365, KOX_16735, KOX_25695, KOX_12270, KOX_15055, KOX_02920, KOX_13330, KOX_09205, KOX_19645, KOX_23415, KOX_16785, KOX_04215, KOX_05500, malS, KOX_06515, KOX_14735, KOX_15150, KOX_18350, KOX_26135, zntB, KOX_07410, KOX_00765, metH, KOX_25845, KOX_23215, KOX_23670, KOX_07500, KOX_12235, KOX_10070, KOX_01110, KOX_01370, KOX_13865, KOX_16945, KOX_16755, rnfD, KOX_26070, KOX_18320, KOX_01470, KOX_03050, KOX_03630, KOX_05300, treF, KOX_16020, KOX_16060, celA, KOX_04160, gltX.

TABLE 5a
List of genes, particularly relating to Klebsiella pneumonia

| | | | | |
|---|---|---|---|---|
| parC | KPN_01607 | gyrA | KPN_02451 | baeR |
| aceF | ybgH | ynjE | KPN_01951 | KPN_01961 |
| KPN_02114 | mhpA | KPN_02128 | KPN_02144 | KPN_02149 |
| ydiJ | btuE | oppC | pth | KPN_02298 |
| KPN_02302 | dadA | yoaA | ftn | cbl |
| hisB | yegQ | yehY | KPN_02580 | yejH |
| KPN_02621 | yfaW | KPN_02170 | KPN_02025 | livG |
| livM | livH | fliY | yedQ | abgB |
| treA | baeS | KPN_02399 | ydcR | anmK |
| ccmF | KPN_02440 | KPN_02540 | KPN_01752 | KPN_04195 |

According to certain embodiments, mutations in at least two, three, four, five, six, seven, eight, nine or ten genes are determined in any of the methods of the present invention, e.g. in at least two genes or in at least three genes. Instead of testing only single genes or mutants, a combination of several variant positions can improve the prediction accuracy and further reduce false positive findings that are influenced by other factors. Therefore, it is in particular preferred to determine the presence of a mutation in 2, 3, 4, 5, 6, 7, 8 or 9 (or more) genes selected from Table 5a and/or Table 5b.

TABLE 5b
List of genes, particularly relating to Klebsiella oxytoca

| | | | | | |
|---|---|---|---|---|---|
| KOX_26125 | KOX_13365 | KOX_16735 | KOX_25695 | KOX_12270 | KOX_15055 |
| KOX_02920 | KOX_13330 | KOX_09205 | KOX_19645 | KOX_23415 | KOX_16785 |
| KOX_04215 | KOX_05500 | malS | KOX_06515 | KOX_14735 | KOX_15150 |
| KOX_18350 | KOX_26135 | zntB | KOX_07410 | KOX_00765 | metH |
| KOX_25845 | KOX_23215 | KOX_23670 | KOX_07500 | KOX_12235 | KOX_10070 |
| KOX_01110 | KOX_01370 | KOX_13865 | KOX_16945 | KOX_16755 | rnfD |
| KOX_26070 | KOX_18320 | KOX_01470 | KOX_03050 | KOX_03630 | KOX_05300 |
| treF | KOX_16020 | KOX_16060 | celA | KOX_04160 | gltX |

According to certain embodiments, the reference genome of Klebsiella, particularly Klebsiella pneumoniae and/or Klebsiella oxytoca, is NC_009648 and/or NC_016612, as annotated at the NCBI. According to certain embodiments, the reference genome of Klebsiella pneumoniae is NC_009648 and the reference genome of Klebsiella oxytoca is NC_016612, as annotated at the NCBI. According to certain embodiments, statistical analysis in the present methods is carried out using Fisher's test with p<10⁻⁶, preferably p<10⁻⁹, particularly p<10⁻¹⁰, particularly p<10⁻¹¹. Also, according to certain embodiments, the method further comprises correlating different genetic sites to each other. Also the other aspects of the embodiments of the first and second aspect of the invention apply.

According to certain embodiments of the method of the twelfth and/or thirteenth aspect of the present invention, as well as also of the eighteenth aspect of the present invention, the antimicrobial drug is an antibiotic. According to certain embodiments, the antibiotic is a lactam antibiotic and a mutation in at least one of the genes listed in Table 6a and/or Table 6b is detected, or a mutation in at least one of the positions (denoted POS in the tables) listed in Table 6a and/or Table 6b.

According to certain embodiments of the method of the twelfth and/or thirteenth aspect of the present invention, as well as also of the eighteenth aspect of the present invention, the Klebsiella species is particularly Klebsiella pneumonia, the antibiotic is a lactam antibiotic, and a mutation in at least one of the genes listed in Table 6a is detected, or a mutation in at least one of the positions (denoted POS in the tables) listed in Table 6a.

According to certain embodiments of the method of the twelfth and/or thirteenth aspect of the present invention, as well as also of the eighteenth aspect of the present invention, the Klebsiella species is particularly Klebsiella oxytoca, the antibiotic is a lactam antibiotic, and a mutation in at least one of the genes listed in Table 6b is detected, or a mutation in at least one of the positions (denoted POS in the tables) listed in Table 6b.

According to certain embodiments of the method of the twelfth and/or thirteenth aspect of the present invention, as well as also of the eighteenth aspect of the present invention, the Klebsiella species is particularly Klebsiella pneumoniae, the antibiotic is at least one of CF, CFT, IMP, CFZ, CRM, ETP, CAX, AZT, P/T, CPE, AM, A/S, CAZ, MER and AUG and a mutation in at least one of the genes of parC, KPN_01607, gyrA, KPN_02451, baeR, aceF, ybgH, ynjE, KPN_01951, KPN_01961, KPN_02114, mhpA, KPN_02128, KPN_02144, KPN_02149 is detected, or a mutation in at least one of the positions of 3763210, 1784305, 1784302, 2905411, 2673906, 2773232, 140517, 809148, 1364586, 2150691, 2159024, 2317024, 2325877, 2331649, 2347930, 2355785.

TABLE 6a
List for lactam antibiotics, particularly for Klebsiella pneumoniae

| | | | p-value | genbank protein |
|---|---|---|---|---|
| gene name | POS | antibiotic | (FDR) | accession number |
| parC | 3763210 | CF; T/S; TE; CFT; LVX; GM; IMP; CFZ; | 1.9784E−152 | YP_001337063.1 |
| | | CRM; ETP; CP; CAX; AZT; P/T; CPE; AM; | | |
| | | A/S; CAZ; TO; MER; AUG | | |
| KPN_01607 | 1784305 | CF; T/S; TE; CFT; LVX; GM; IMP; CFZ; | 1.5316E−115 | YP_001335268.1 |
| | | CRM; ETP; CP; CAX; AZT; P/T; CPE; AM; | | |
| | | A/S; CAZ; TO; MER; AUG | | |
| KPN_01607 | 1784302 | CF; T/S; TE; CFT; LVX; GM; IMP; CFZ; | 8.1983E−115 | YP_001335268.1 |
| | | CRM; ETP; CP; CAX; AZT; P/T; CPE; AM; | | |
| | | A/S; CAZ; TO; MER; AUG | | |
| gyrA | 2905411 | CF; T/S; TE; CFT; LVX; GM; IMP; CFZ; | 4.3727E−106 | YP_001336287.1 |
| | | CRM; ETP; CP; CAX; AZT; P/T; CPE; AM; | | |
| | | A/S; CAZ; TO; MER; AUG | | |
| KPN_02451 | 2673906 | CF; T/S; TE; CFT; LVX; GM; IMP; CFZ; | 5.0133E−104 | YP_001336099.1 |
| | | CRM; ETP; CP; CAX; AZT; P/T; CPE; AM; | | |
| | | A/S; CAZ; TO; MER; AUG | | |
| baeR | 2773232 | CF; T/S; TE; CFT; LVX; GM; IMP; CFZ; | 5.5237E−104 | YP_001336179.1 |
| | | CRM; ETP; CP; CAX; AZT; P/T; CPE; AM; | | |
| | | A/S; CAZ; TO; MER; AUG | | |
| aceF | 140517 | CF; T/S; TE; CFT; LVX; GM; IMP; CFZ; | 1.3942E−103 | YP_001333809.1 |
| | | CRM; ETP; CP; CAX; AZT; P/T; CPE; AM; | | |
| | | A/S; CAZ; TO; MER; AUG | | |
| ybgH | 809148 | CF; T/S; TE; CFT; LVX; GM; IMP; CFZ; | 1.3942E−103 | YP_001334393.1 |
| | | CRM; ETP; CP; CAX; AZT; P/T; CPE; AM; | | |
| | | A/S; CAZ; TO; MER; AUG | | |
| ynjE | 1364586 | CF; T/S; TE; CFT; LVX; GM; IMP; CFZ; | 1.3942E−103 | YP_001334876.1 |
| | | CRM; ETP; CP; CAX; AZT; P/T; CPE; AM; | | |
| | | A/S; CAZ; TO; MER; AUG | | |
| KPN_01951 | 2150691 | CF; T/S; TE; CFT; LVX; GM; IMP; CFZ; | 1.3942E−103 | YP_001335612.1 |
| | | CRM; ETP; CP; CAX; AZT; P/T; CPE; AM; | | |
| | | A/S; CAZ; TO; MER; AUG | | |
| KPN_01961 | 2159024 | CF; T/S; TE; CFT; LVX; GM; IMP; CFZ; | 1.3942E−103 | YP_001335622.1 |
| | | CRM; ETP; CP; CAX; AZT; P/T; CPE; AM; | | |
| | | A/S; CAZ; TO; MER; AUG | | |
| KPN_02114 | 2317024 | CF; T/S; TE; CFT; LVX; GM; IMP; CFZ; | 1.3942E−103 | YP_001335772.1 |
| | | CRM; ETP; CP; CAX; AZT; P/T; CPE; AM; | | |
| | | A/S; CAZ; TO; MER; AUG | | |
| mhpA | 2325877 | CF; T/S; TE; CFT; LVX; GM; IMP; CFZ; | 1.3942E−103 | YP_001335780.1 |
| | | CRM; ETP; CP; CAX; AZT; P/T; CPE; AM; | | |
| | | A/S; CAZ; TO; MER; AUG | | |
| KPN_02128 | 2331649 | CF; T/S; TE; CFT; LVX; GM; IMP; CFZ; | 1.3942E−103 | YP_001335786.1 |
| | | CRM; ETP; CP; CAX; AZT; P/T; CPE; AM; | | |
| | | A/S; CAZ; TO; MER; AUG | | |
| KPN_02144 | 2347930 | CF; T/S; TE; CFT; LVX; GM; IMP; CFZ; | 1.3942E−103 | YP_001335802.1 |
| | | CRM; ETP; CP; CAX; AZT; P/T; CPE; AM; | | |
| | | A/S; CAZ; TO; MER; AUG | | |
| KPN_02149 | 2355785 | CF; T/S; TE; CFT; LVX; GM; IMP; CFZ; | 1.3942E−103 | YP_001335807.1 |
| | | CRM; ETP; CP; CAX; AZT; P/T; CPE; AM; | | |
| | | A/S; CAZ; TO; MER; AUG | | |

FDR: determined according to FDR (Benjamini Hochberg) method (Benjamini Hochberg, 1995)

TABLE 6b
List for lactam antibiotics, particularly for Klebsiella oxytoca

| | | | p-value | genbank protein |
|---|---|---|---|---|
| gene name | POS | antibiotic | (FDR) | accession number |
| KOX_26125 | 5645611 | CF; T/S; TE; CFT; CFZ; CRM; CP; CAX; | 6.03526E−61 | YP_005021173.1 |
| | | AZT; P/T; CPE; AM; A/S; CAZ; LVX; | | |
| | | AUG | | |
| KOX_02920 | 617510 | CF; T/S; CP; CFT; CFZ; CRM; AZT; | 9.66285E−19 | YP_005016564.1 |
| | | P/T; CPE; A/S; CAZ; LVX; AUG | | |
| KOX_13330 | 2880820 | CF; TE; CFT; CFZ; CRM; CP; CAX; AZT; | 5.55043E−17 | YP_005018629.1 |
| | | P/T; LVX; A/S; AUG | | |
| KOX_09205 | 1955164 | CF; CP; CFT; CFZ; CRM; CAX; AZT; | 1.26701E−15 | YP_005017806.1 |
| | | P/T; LVX; AM; A/S | | |
| KOX_19645 | 4247719 | CF; T/S; A/S; CRM; CAX; P/T; LVX; | 7.88021E−35 | YP_005019890.1 |
| | | AM; CFZ; AUG | | |
| KOX_23415 | 5051859 | CF; T/S; CP; A/S; CRM; CAX; AZT; | 4.4648E−16 | YP_005020638.1 |
| | | P/T; CPE; LVX; AUG | | |
| KOX_16785 | 3642225 | CF; T/S; CFZ; CRM; CAX; P/T; CPE; | 1.94883E−14 | YP_005019318.1 |
| | | A/S; LVX; AUG | | |
| KOX_04215 | 883865 | CF; CFZ; CRM; AZT; AM; A/S; AUG | 1.50603E−40 | YP_005016819.1 |
| KOX_05500 | 1144432 | CF; CFZ; CRM; AZT; AM; A/S; AUG | 1.50603E−40 | YP_005017074.1 |
| malS | 1180202 | CF; CFZ; CRM; AZT; AM; A/S; AUG | 1.50603E−40 | YP_005017105.1 |
| KOX_06515 | 1357618 | CF; CFZ; CRM; AZT; AM; A/S; AUG | 1.50603E−40 | YP_005017272.1 |
| KOX_14735 | 3195636 | CF; CFZ; CRM; AZT; AM; A/S; AUG | 1.50603E−40 | YP_005018910.1 |
| KOX_15150 | 3282908 | CF; CFZ; CRM; AZT; AM; A/S; AUG | 1.50603E−40 | YP_005018993.1 |
| KOX_18350 | 3969498 | CF; CFZ; CRM; AZT; AM; A/S; AUG | 1.50603E−40 | YP_005019631.1 |
| KOX_26135 | 5648918 | CF; CFZ; CRM; AZT; AM; A/S; AUG | 1.50603E−40 | YP_005021175.1 |
| gltX | 5786658 | CF; CFZ; CRM; AZT; AM; A/S; AUG | 1.50603E−40 | YP_005021305.1 |

According to certain embodiments of the method of the twelfth and/or thirteenth aspect of the present invention, as well as also of the eighteenth aspect of the present invention, the Klebsiella species is particularly Klebsiella oxytoca, the antibiotic is at least one of CF, CRM and A/S and a mutation in at least one of the genes of KOX_26125, KOX_02920, KOX_13330, KOX_09205, KOX_19645, KOX_23415, KOX_16785, KOX_04215, KOX_05500, malS, KOX_06515, KOX_14735, KOX_15150, KOX_18350, KOX_26135, gltX is detected, or a mutation in at least one of the positions of 5645611, 617510, 2880820, 1955164, 4247719, 5051859, 3642225, 883865, 1144432, 1180202, 1357618, 3195636, 3282908, 3969498, 5648918, 5786658.

According to certain embodiments of the method of the twelfth and/or thirteenth aspect of the present invention, as well as also of the eighteenth aspect of the present invention, the Klebsiella species is particularly Klebsiella oxytoca, the antibiotic is CFZ and a mutation in at least one of the genes of KOX_26125, KOX_02920, KOX_13330, KOX_09205, KOX_19645, KOX_16785, KOX_04215, KOX_05500, malS, KOX_06515, KOX_14735, KOX_15150, KOX_18350, KOX_26135, gltX is detected, or a mutation in at least one of the positions of 5645611, 617510, 2880820, 1955164, 4247719, 3642225, 883865, 1144432, 1180202, 1357618, 3195636, 3282908, 3969498, 5648918, 5786658.

According to certain embodiments of the method of the twelfth and/or thirteenth aspect of the present invention, as well as also of the eighteenth aspect of the present invention, the Klebsiella species is particularly Klebsiella oxytoca, the antibiotic is AZT and a mutation in at least one of the genes of KOX_26125, KOX_02920, KOX_13330, KOX_09205, KOX_23415, KOX_04215, KOX_05500, malS, KOX_06515, KOX_14735, KOX_15150, KOX_18350, KOX_26135, gltX is detected, or a mutation in at least one of the positions of 5645611, 617510, 2880820, 1955164, 5051859, 883865, 1144432, 1180202, 1357618, 3195636, 3282908, 3969498, 5648918, 5786658.

According to certain embodiments of the method of the twelfth and/or thirteenth aspect of the present invention, as well as also of the eighteenth aspect of the present invention, the Klebsiella species is particularly Klebsiella oxytoca, the antibiotic is AM and a mutation in at least one of the genes of KOX_26125, KOX_09205, KOX_19645, KOX_04215, KOX_05500, malS, KOX_06515, KOX_14735, KOX_15150, KOX_18350, KOX_26135, gltX is detected, or a mutation in at least one of the positions of 5645611, 1955164, 4247719, 883865, 1144432, 1180202, 1357618, 3195636, 3282908, 3969498, 5648918, 5786658.

According to certain embodiments of the method of the twelfth and/or thirteenth aspect of the present invention, as well as also of the eighteenth aspect of the present invention, the Klebsiella species is particularly Klebsiella oxytoca, the antibiotic is AUG and a mutation in at least one of the genes of KOX_26125, KOX_02920, KOX_13330, KOX_19645, KOX_23415, KOX_16785, KOX_04215, KOX_05500, malS, KOX_06515, KOX_14735, KOX_15150, KOX_18350, KOX_26135, gltX is detected, or a mutation in at least one of the positions of 5645611, 617510, 2880820, 4247719, 5051859, 3642225, 883865, 1144432, 1180202, 1357618, 3195636, 3282908, 3969498, 5648918, 5786658.

According to certain embodiments of the method of the twelfth and/or thirteenth aspect of the present invention, as well as also of the eighteenth aspect of the present invention, the Klebsiella species is particularly Klebsiella oxytoca, the antibiotic is P/T and a mutation in at least one of the genes of KOX_26125, KOX_02920, KOX_13330, KOX_09205, KOX_19645, KOX_23415, KOX_16785 is detected, or a mutation in at least one of the positions of 5645611, 617510, 2880820, 1955164, 4247719, 5051859, 3642225.

According to certain embodiments of the method of the twelfth and/or thirteenth aspect of the present invention, as well as also of the eighteenth aspect of the present invention, the Klebsiella species is particularly Klebsiella oxytoca, the antibiotic is CAX and a mutation in at least one of the genes of KOX_26125, KOX_13330, KOX_09205, KOX_19645, KOX_23415, KOX_16785 is detected, or a mutation in at least one of the positions of 5645611, 2880820, 1955164, 4247719, 5051859, 3642225.

According to certain embodiments of the method of the twelfth and/or thirteenth aspect of the present invention, as well as also of the eighteenth aspect of the present invention, the Klebsiella species is particularly Klebsiella oxytoca, the antibiotic is CPE and a mutation in at least one of the genes of KOX_26125, KOX_02920, KOX_23415, KOX_16785 is detected, or a mutation in at least one of the positions of 5645611, 617510, 5051859, 3642225.

According to certain embodiments of the method of the twelfth and/or thirteenth aspect of the present invention, as well as also of the eighteenth aspect of the present invention, the Klebsiella species is particularly Klebsiella oxytoca, the antibiotic is CFT and a mutation in at least one of the genes of KOX_26125, KOX_02920, KOX_13330, KOX_09205 is detected, or a mutation in at least one of the positions of 5645611, 617510, 2880820, 1955164.

According to certain embodiments of the method of the twelfth and/or thirteenth aspect of the present invention, as well as also of the eighteenth aspect of the present invention, the Klebsiella species is particularly Klebsiella oxytoca, the antibiotic is CAZ and a mutation in at least one of the genes of KOX_26125, KOX_02920 is detected, or a mutation in at least one of the positions of 5645611, 617510.

According to certain embodiments of the method of the twelfth and/or thirteenth aspect of the present invention, as well as also of the eighteenth aspect of the present invention, the antibiotic is a quinolone antibiotic, particularly a fluoroquinolone antibiotic, and a mutation in at least one of the genes listed in Table 7a and/or Table 7b is detected, or a mutation in at least one of the positions (denoted POS in the tables) listed in Table 7a and/or Table 7b.

TABLE 7a
List for quinolone antibiotics, particularly for Klebsiella pneumoniae

| | | | p-value | genbank protein |
|---|---|---|---|---|
| gene name | POS | antibiotic | (FDR) | accession number |
| parC | 3763210 | CF; T/S; TE; CFT; LVX; GM; IMP; CFZ; | 1.9784E−152 | YP_001337063.1 |
| | | CRM; ETP; CP; CAX; AZT; P/T; CPE; AM; | | |
| | | A/S; CAZ; TO; MER; AUG | | |
| KPN_01607 | 1784305 | CF; T/S; TE; CFT; LVX; GM; IMP; CFZ; | 1.5316E−115 | YP_001335268.1 |
| | | CRM; ETP; CP; CAX; AZT; P/T; CPE; AM; | | |
| | | A/S; CAZ; TO; MER; AUG | | |
| KPN_01607 | 1784302 | CF; T/S; TE; CFT; LVX; GM; IMP; CFZ; | 8.1983E−115 | YP_001335268.1 |
| | | CRM; ETP; CP; CAX; AZT; P/T; CPE; AM; | | |
| | | A/S; CAZ; TO; MER; AUG | | |
| gyrA | 2905411 | CF; T/S; TE; CFT; LVX; GM; IMP; CFZ; | 4.3727E−106 | YP_001336287.1 |
| | | CRM; ETP; CP; CAX; AZT; P/T; CPE; AM; | | |
| | | A/S; CAZ; TO; MER; AUG | | |
| KPN_02451 | 2673906 | CF; T/S; TE; CFT; LVX; GM; IMP; CFZ; | 5.0133E−104 | YP_001336099.1 |
| | | CRM; ETP; CP; CAX; AZT; P/T; CPE; AM; | | |
| | | A/S; CAZ; TO; MER; AUG | | |
| baeR | 2773232 | CF; T/S; TE; CFT; LVX; GM; IMP; CFZ; | 5.5237E−104 | YP_001336179.1 |
| | | CRM; ETP; CP; CAX; AZT; P/T; CPE; AM; | | |
| | | A/S; CAZ; TO; MER; AUG | | |
| aceF | 140517 | CF; T/S; TE; CFT; LVX; GM; IMP; CFZ; | 1.3942E−103 | YP_001333809.1 |
| | | CRM; ETP; CP; CAX; AZT; P/T; CPE; AM; | | |
| | | A/S; CAZ; TO; MER; AUG | | |
| ybgH | 809148 | CF; T/S; TE; CFT; LVX; GM; IMP; CFZ; | 1.3942E−103 | YP_001334393.1 |
| | | CRM; ETP; CP; CAX; AZT; P/T; CPE; AM; | | |
| | | A/S; CAZ; TO; MER; AUG | | |
| ynjE | 1364586 | CF; T/S; TE; CFT; LVX; GM; IMP; CFZ; | 1.3942E−103 | YP_001334876.1 |
| | | CRM; ETP; CP; CAX; AZT; P/T; CPE; AM; | | |
| | | A/S; CAZ; TO; MER; AUG | | |
| KPN_01951 | 2150691 | CF; T/S; TE; CFT; LVX; GM; IMP; CFZ; | 1.3942E−103 | YP_001335612.1 |
| | | CRM; ETP; CP; CAX; AZT; P/T; CPE; AM; | | |
| | | A/S; CAZ; TO; MER; AUG | | |
| KPN_01961 | 2159024 | CF; T/S; TE; CFT; LVX; GM; IMP; CFZ; | 1.3942E−103 | YP_001335622.1 |
| | | CRM; ETP; CP; CAX; AZT; P/T; CPE; AM; | | |
| | | A/S; CAZ; TO; MER; AUG | | |
| KPN_02114 | 2317024 | CF; T/S; TE; CFT; LVX; GM; IMP; CFZ; | 1.3942E−103 | YP_001335772.1 |
| | | CRM; ETP; CP; CAX; AZT; P/T; CPE; AM; | | |
| | | A/S; CAZ; TO; MER; AUG | | |
| mhpA | 2325877 | CF; T/S; TE; CFT; LVX; GM; IMP; CFZ; | 1.3942E−103 | YP_001335780.1 |
| | | CRM; ETP; CP; CAX; AZT; P/T; CPE; AM; | | |
| | | A/S; CAZ; TO; MER; AUG | | |
| KPN_02128 | 2331649 | CF; T/S; TE; CFT; LVX; GM; IMP; CFZ; | 1.3942E−103 | YP_001335786.1 |
| | | CRM; ETP; CP; CAX; AZT; P/T; CPE; AM; | | |
| | | A/S; CAZ; TO; MER; AUG | | |
| KPN_02144 | 2347930 | CF; T/S; TE; CFT; LVX; GM; IMP; CFZ; | 1.3942E−103 | YP_001335802.1 |
| | | CRM; ETP; CP; CAX; AZT; P/T; CPE; AM; | | |
| | | A/S; CAZ; TO; MER; AUG | | |
| KPN_02149 | 2355785 | CF; T/S; TE; CFT; LVX; GM; IMP; CFZ; | 1.3942E−103 | YP_001335807.1 |
| | | CRM; ETP; CP; CAX; AZT; P/T; CPE; AM; | | |
| | | A/S; CAZ; TO; MER; AUG | | |

According to certain embodiments of the method of the twelfth and/or thirteenth aspect of the present invention, as well as also of the eighteenth aspect of the present invention, the Klebsiella species is particularly Klebsiella pneumonia, the antibiotic is a quinolone antibiotic, and a mutation in at least one of the genes listed in Table 7a is detected, or a mutation in at least one of the positions (denoted POS in the tables) listed in Table 7a.

According to certain embodiments of the method of the twelfth and/or thirteenth aspect of the present invention, as well as also of the eighteenth aspect of the present invention, the Klebsiella species is particularly Klebsiella oxytoca, the antibiotic is a quinolone antibiotic, and a mutation in at least one of the genes listed in Table 7b is detected, or a mutation in at least one of the positions (denoted POS in the tables) listed in Table 7b.

According to certain embodiments of the method of the twelfth and/or thirteenth aspect of the present invention, as well as also of the eighteenth aspect of the present invention, the Klebsiella species is particularly Klebsiella pneumoniae, the antibiotic is at least one of CP and LVX and a mutation in at least one of the genes of parC, KPN_01607, gyrA, KPN_02451, baeR, aceF, ybgH, ynjE, KPN_01951, KPN_01961, KPN_02114, mhpA, KPN_02128, KPN_02144, KPN_02149 is detected, or a mutation in at least one of the positions of 3763210, 1784305, 1784302, 2905411, 2673906, 2773232, 140517, 809148, 1364586, 2150691, 2159024, 2317024, 2325877, 2331649, 2347930, 2355785.

TABLE 7b
List for quinolone antibiotics, particularly for Klebsiella oxytoca

| | | | p-value | genbank protein |
|---|---|---|---|---|
| gene name | POS | antibiotic | (FDR) | accession number |
| KOX_26125 | 5645611 | CF; T/S; TE; CFT; CFZ; CRM; CP; CAX; | 6.03526E−61 | YP_005021173.1 |
| | | AZT; P/T; CPE; AM; A/S; CAZ; LVX; | | |
| | | AUG | | |
| KOX_02920 | 617510 | CF; T/S; CP; CFT; CFZ; CRM; AZT; P/T; | 9.66285E−19 | YP_005016564.1 |
| | | CPE; A/S; CAZ; LVX; AUG | | |
| zntB | 4112732 | CF; CP; CFZ; CRM; LVX; AM | 2.39757E−18 | YP_005019768.1 |
| KOX_07410 | 1552287 | CF; CP; CFZ; AZT; LVX; A/S | 4.43464E−18 | YP_005017451.1 |
| KOX_00765 | 168216 | CF; CP; CFZ; CRM; LVX; A/S | 1.29812E−17 | YP_005016137.1 |
| metH | 1719218 | CF; CP; CFZ; CRM; LVX; AM; A/S | 5.55043E−17 | YP_005017580.1 |
| KOX_13330 | 2880820 | CF; TE; CFT; CFZ; CRM; CP; CAX; AZT; | 5.55043E−17 | YP_005018629.1 |
| | | P/T; LVX; A/S; AUG | | |
| KOX_25845 | 5578458 | CF; CP; CFZ; CRM; LVX; A/S | 6.47328E−17 | YP_005021119.1 |
| KOX_23215 | 5005193 | CF; CFZ; CP; LVX; A/S | 8.6943E−17 | YP_005020598.1 |
| KOX_23670 | 5109476 | CF; CP; CFZ; LVX; AM; A/S | 1.05494E−16 | YP_005020689.1 |
| KOX_07500 | 1577171 | CF; CP; CFZ; CRM; AZT; LVX; A/S; AUG | 1.19133E−16 | YP_005017469.1 |
| KOX_12235 | 2642791 | CF; CP; CFZ; CRM; LVX; A/S | 1.30523E−16 | YP_005018412.1 |
| KOX_10070 | 2149606 | CF; CP; CFZ; LVX; AM; A/S | 2.33948E−16 | YP_005017979.1 |
| KOX_01110 | 237416 | CF; CP; CFZ; CRM; LVX; A/S | 2.36704E−16 | YP_005016206.1 |

According to certain embodiments of the method of the twelfth and/or thirteenth aspect of the present invention, as well as also of the eighteenth aspect of the present invention, the Klebsiella species is particularly Klebsiella oxytoca, the antibiotic is at least one of CP and LVX and a mutation in at least one of the genes of KOX_26125, KOX_02920, zntB, KOX_07410, KOX_00765, metH, KOX_13330, KOX_25845, KOX_23215, KOX_23670, KOX_07500, KOX_12235, KOX_10070, KOX_01110 is detected, or a mutation in at least one of the positions of 5645611, 617510, 4112732, 1552287, 168216, 1719218, 2880820, 5578458, 5005193, 5109476, 1577171, 2642791, 2149606, 237416.

According to certain embodiments of the method of the twelfth and/or thirteenth aspect of the present invention, as well as also of the eighteenth aspect of the present invention, the antibiotic is an aminoglycoside antibiotic and a mutation in at least one of the genes listed in Table 8 is detected, or a mutation in at least one of the positions (denoted POS in the tables) listed in Table 8, wherein the Klebsiella species is particularly Klebsiella pneumoniae.

TABLE 8
List for aminoglycoside antibiotics, particularly for Klebsiella pneumoniae

| | | | p-value | genbank protein |
|---|---|---|---|---|
| gene name | POS | antibiotic | (FDR) | accession number |
| parC | 3763210 | CF; T/S; TE; CFT; LVX; GM; IMP; CFZ; | 1.9784E−152 | YP_001337063.1 |
| | | CRM; ETP; CP; CAX; AZT; P/T; CPE; AM; | | |
| | | A/S; CAZ; TO; MER; AUG | | |
| KPN_01607 | 1784305 | CF; T/S; TE; CFT; LVX; GM; IMP; CFZ; | 1.5316E−115 | YP_001335268.1 |
| | | CRM; ETP; CP; CAX; AZT; P/T; CPE; AM; | | |
| | | A/S; CAZ; TO; MER; AUG | | |
| KPN_01607 | 1784302 | CF; T/S; TE; CFT; LVX; GM; IMP; CFZ; | 8.1983E−115 | YP_001335268.1 |
| | | CRM; ETP; CP; CAX; AZT; P/T; CPE; AM; | | |
| | | A/S; CAZ; TO; MER; AUG | | |
| gyrA | 2905411 | CF; T/S; TE; CFT; LVX; GM; IMP; CFZ; | 4.3727E−106 | YP_001336287.1 |
| | | CRM; ETP; CP; CAX; AZT; P/T; CPE; AM; | | |
| | | A/S; CAZ; TO; MER; AUG | | |
| KPN_02451 | 2673906 | CF; T/S; TE; CFT; LVX; GM; IMP; CFZ; | 5.0133E−104 | YP_001336099.1 |
| | | CRM; ETP; CP; CAX; AZT; P/T; CPE; AM; | | |
| | | A/S; CAZ; TO; MER; AUG | | |
| baeR | 2773232 | CF; T/S; TE; CFT; LVX; GM; IMP; CFZ; | 5.5237E−104 | YP_001336179.1 |
| | | CRM; ETP; CP; CAX; AZT; P/T; CPE; AM; | | |
| | | A/S; CAZ; TO; MER; AUG | | |
| aceF | 140517 | CF; T/S; TE; CFT; LVX; GM; IMP; CFZ; | 1.3942E−103 | YP_001333809.1 |
| | | CRM; ETP; CP; CAX; AZT; P/T; CPE; AM; | | |
| | | A/S; CAZ; TO; MER; AUG | | |
| ybgH | 809148 | CF; T/S; TE; CFT; LVX; GM; IMP; CFZ; | 1.3942E−103 | YP_001334393.1 |
| | | CRM; ETP; CP; CAX; AZT; P/T; CPE; AM; | | |
| | | A/S; CAZ; TO; MER; AUG | | |
| ynjE | 1364586 | CF; T/S; TE; CFT; LVX; GM; IMP; CFZ; | 1.3942E−103 | YP_001334876.1 |
| | | CRM; ETP; CP; CAX; AZT; P/T; CPE; AM; | | |
| | | A/S; CAZ; TO; MER; AUG | | |
| KPN_01951 | 2150691 | CF; T/S; TE; CFT; LVX; GM; IMP; CFZ; | 1.3942E−103 | YP_001335612.1 |
| | | CRM; ETP; CP; CAX; AZT; P/T; CPE; AM; | | |
| | | A/S; CAZ; TO; MER; AUG | | |
| KPN_01961 | 2159024 | CF; T/S; TE; CFT; LVX; GM; IMP; CFZ; | 1.3942E−103 | YP_001335622.1 |
| | | CRM; ETP; CP; CAX; AZT; P/T; CPE; AM; | | |
| | | A/S; CAZ; TO; MER; AUG | | |
| KPN_02114 | 2317024 | CF; T/S; TE; CFT; LVX; GM; IMP; CFZ; | 1.3942E−103 | YP_001335772.1 |
| | | CRM; ETP; CP; CAX; AZT; P/T; CPE; AM; | | |
| | | A/S; CAZ; TO; MER; AUG | | |
| mhpA | 2325877 | CF; T/S; TE; CFT; LVX; GM; IMP; CFZ; | 1.3942E−103 | YP_001335780.1 |
| | | CRM; ETP; CP; CAX; AZT; P/T; CPE; AM; | | |
| | | A/S; CAZ; TO; MER; AUG | | |
| KPN_02128 | 2331649 | CF; T/S; TE; CFT; LVX; GM; IMP; CFZ; | 1.3942E−103 | YP_001335786.1 |
| | | CRM; ETP; CP; CAX; AZT; P/T; CPE; AM; | | |
| | | A/S; CAZ; TO; MER; AUG | | |
| KPN_02144 | 2347930 | CF; T/S; TE; CFT; LVX; GM; IMP; CFZ; | 1.3942E−103 | YP_001335802.1 |
| | | CRM; ETP; CP; CAX; AZT; P/T; CPE; AM; | | |
| | | A/S; CAZ; TO; MER; AUG | | |
| KPN_02149 | 2355785 | CF; T/S; TE; CFT; LVX; GM; IMP; CFZ; | 1.3942E−103 | YP_001335807.1 |
| | | CRM; ETP; CP; CAX; AZT; P/T; CPE; AM; | | |
| | | A/S; CAZ; TO; MER; AUG | | |

According to certain embodiments of the method of the twelfth and/or thirteenth aspect of the present invention, as well as also of the eighteenth aspect of the present invention, the Klebsiella species is particularly Klebsiella pneumoniae, the antibiotic is at least one of GM and TO and a mutation in at least one of the genes of parC, KPN_01607, gyrA, KPN_02451, baeR, aceF, ybgH, ynjE, KPN_01951, KPN_01961, KPN_02114, mhpA, KPN_02128, KPN_02144, KPN_02149 is detected, or a mutation in at least one of the positions of 3763210, 1784305, 1784302, 2905411, 2673906, 2773232, 140517, 809148, 1364586, 2150691, 2159024, 2317024, 2325877, 2331649, 2347930, 2355785.

According to certain embodiments of the method of the twelfth and/or thirteenth aspect of the present invention, as well as also of the eighteenth aspect of the present invention, the antibiotic is a polyketide antibiotic, particularly a tetracycline antibiotic, and a mutation in at least one of the genes listed in Table 9a and/or Table 9b is detected, or a mutation in at least one of the positions (denoted POS in the tables) listed in Table 9a and/or Table 9b.

According to certain embodiments of the method of the twelfth and/or thirteenth aspect of the present invention, as well as also of the eighteenth aspect of the present invention, the Klebsiella species is particularly Klebsiella pneumonia, the antibiotic is a polyketide antibiotic, particularly a tetracycline antibiotic, and a mutation in at least one of the genes listed in Table 9a is detected, or a mutation in at least one of the positions (denoted POS in the tables) listed in Table 9a.

TABLE 9a
List for polyketide antibiotics, particularly for Klebsiella pneumoniae

| | | | p-value | genbank protein |
|---|---|---|---|---|
| gene name | POS | antibiotic | (FDR) | accession number |
| parC | 3763210 | CF; T/S; TE; CFT; LVX; GM; IMP; CFZ; | 1.9784E−152 | YP_001337063.1 |
| | | CRM; ETP; CP; CAX; AZT; P/T; CPE; AM; | | |
| | | A/S; CAZ; TO; MER; AUG | | |
| KPN_01607 | 1784305 | CF; T/S; TE; CFT; LVX; GM; IMP; CFZ; | 1.5316E−115 | YP_001335268.1 |
| | | CRM; ETP; CP; CAX; AZT; P/T; CPE; AM; | | |
| | | A/S; CAZ; TO; MER; AUG | | |
| KPN_01607 | 1784302 | CF; T/S; TE; CFT; LVX; GM; IMP; CFZ; | 8.1983E−115 | YP_001335268.1 |
| | | CRM; ETP; CP; CAX; AZT; P/T; CPE; AM; | | |
| | | A/S; CAZ; TO; MER; AUG | | |
| gyrA | 2905411 | CF; T/S; TE; CFT; LVX; GM; IMP; CFZ; | 4.3727E−106 | YP_001336287.1 |
| | | CRM; ETP; CP; CAX; AZT; P/T; CPE; AM; | | |
| | | A/S; CAZ; TO; MER; AUG | | |
| KPN_02451 | 2673906 | CF; T/S; TE; CFT; LVX; GM; IMP; CFZ; | 5.0133E−104 | YP_001336099.1 |
| | | CRM; ETP; CP; CAX; AZT; P/T; CPE; AM; | | |
| | | A/S; CAZ; TO; MER; AUG | | |
| baeR | 2773232 | CF; T/S; TE; CFT; LVX; GM; IMP; CFZ; | 5.5237E−104 | YP_001336179.1 |
| | | CRM; ETP; CP; CAX; AZT; P/T; CPE; AM; | | |
| | | A/S; CAZ; TO; MER; AUG | | |
| aceF | 140517 | CF; T/S; TE; CFT; LVX; GM; IMP; CFZ; | 1.3942E−103 | YP_001333809.1 |
| | | CRM; ETP; CP; CAX; AZT; P/T; CPE; AM; | | |
| | | A/S; CAZ; TO; MER; AUG | | |
| ybgH | 809148 | CF; T/S; TE; CFT; LVX; GM; IMP; CFZ; | 1.3942E−103 | YP_001334393.1 |
| | | CRM; ETP; CP; CAX; AZT; P/T; CPE; AM; | | |
| | | A/S; CAZ; TO; MER; AUG | | |
| ynjE | 1364586 | CF; T/S; TE; CFT; LVX; GM; IMP; CFZ; | 1.3942E−103 | YP_001334876.1 |
| | | CRM; ETP; CP; CAX; AZT; P/T; CPE; AM; | | |
| | | A/S; CAZ; TO; MER; AUG | | |
| KPN_01951 | 2150691 | CF; T/S; TE; CFT; LVX; GM; IMP; CFZ; | 1.3942E−103 | YP_001335612.1 |
| | | CRM; ETP; CP; CAX; AZT; P/T; CPE; AM; | | |
| | | A/S; CAZ; TO; MER; AUG | | |
| KPN_01961 | 2159024 | CF; T/S; TE; CFT; LVX; GM; IMP; CFZ; | 1.3942E−103 | YP_001335622.1 |
| | | CRM; ETP; CP; CAX; AZT; P/T; CPE; AM; | | |
| | | A/S; CAZ; TO; MER; AUG | | |
| KPN_02114 | 2317024 | CF; T/S; TE; CFT; LVX; GM; IMP; CFZ; | 1.3942E−103 | YP_001335772.1 |
| | | CRM; ETP; CP; CAX; AZT; P/T; CPE; AM; | | |
| | | A/S; CAZ; TO; MER; AUG | | |
| mhpA | 2325877 | CF; T/S; TE; CFT; LVX; GM; IMP; CFZ; | 1.3942E−103 | YP_001335780.1 |
| | | CRM; ETP; CP; CAX; AZT; P/T; CPE; AM; | | |
| | | A/S; CAZ; TO; MER; AUG | | |
| KPN_02128 | 2331649 | CF; T/S; TE; CFT; LVX; GM; IMP; CFZ; | 1.3942E−103 | YP_001335786.1 |
| | | CRM; ETP; CP; CAX; AZT; P/T; CPE; AM; | | |
| | | A/S; CAZ; TO; MER; AUG | | |
| KPN_02144 | 2347930 | CF; T/S; TE; CFT; LVX; GM; IMP; CFZ; | 1.3942E−103 | YP_001335802.1 |
| | | CRM; ETP; CP; CAX; AZT; P/T; CPE; AM; | | |
| | | A/S; CAZ; TO; MER; AUG | | |
| KPN_02149 | 2355785 | CF; T/S; TE; CFT; LVX; GM; IMP; CFZ; | 1.3942E−103 | YP_001335807.1 |
| | | CRM; ETP; CP; CAX; AZT; P/T; CPE; AM; | | |
| | | A/S; CAZ; TO; MER; AUG | | |

According to certain embodiments of the method of the twelfth and/or thirteenth aspect of the present invention, as well as also of the eighteenth aspect of the present invention, the Klebsiella species is particularly Klebsiella oxytoca, the antibiotic is a polyketide antibiotic, particularly a tetracycline antibiotic, and a mutation in at least one of the genes listed in Table 9b is detected, or a mutation in at least one of the positions (denoted POS in the tables) listed in Table 9b.

TABLE 9b
List for polyketide antibiotics, particularly for Klebsiella oxytoca

| | | | p-value | genbank protein |
|---|---|---|---|---|
| gene name | POS | antibiotic | (FDR) | accession number |
| KOX_26125 | 5645611 | CF; T/S; TE; CFT; CFZ; CRM; CP; | 6.03526E−61 | YP_005021173.1 |
| | | CAX; AZT; P/T; CPE; AM; A/S; CAZ; | | |
| | | LVX; AUG | | |
| KOX_13330 | 2880820 | CF; TE; CFT; CFZ; CRM; CP; CAX; | 5.55043E−17 | YP_005018629.1 |
| | | AZT; P/T; LVX; A/S; AUG | | |
| KOX_13865 | 3001328 | CF; TE; CFZ; CRM; CP; CAX; AZT; | 2.74431E−15 | YP_005018736.1 |
| | | P/T; LVX; A/S | | |
| KOX_16945 | 3678273 | CF; TE; CFZ; CRM; CP; CAX; LVX; A/S | 5.0436E−15 | YP_005019350.1 |
| KOX_16755 | 3636466 | CF; TE; CFZ; CRM; CP; CAX; LVX; A/S | 1.1009E−14 | YP_005019312.1 |
| rnfD | 4740803 | CF; TE; CFZ; CRM; CP; CAX; P/T; LVX; | 1.45289E−14 | YP_005020357.1 |
| | | A/S | | |
| KOX_26070 | 5626010 | CF; CFZ; TE; CRM; A/S | 1.35149E−13 | YP_005021162.1 |

According to certain embodiments of the method of the twelfth and/or thirteenth aspect of the present invention, as well as also of the eighteenth aspect of the present invention, the Klebsiella species is particularly Klebsiella pneumoniae, the antibiotic is TE and a mutation in at least one of the genes of parC, KPN_01607, gyrA, KPN_02451, baeR, aceF, ybgH, ynjE, KPN_01951, KPN_01961, KPN_02114, mhpA, KPN_02128, KPN_02144, KPN_02149 is detected, or a mutation in at least one of the positions of 3763210, 1784305, 1784302, 2905411, 2673906, 2773232, 140517, 809148, 1364586, 2150691, 2159024, 2317024, 2325877, 2331649, 2347930, 2355785.

According to certain embodiments of the method of the twelfth and/or thirteenth aspect of the present invention, as well as also of the eighteenth aspect of the present invention, the Klebsiella species is particularly Klebsiella oxytoca, the antibiotic is TE and a mutation in at least one of the genes of KOX_26125, KOX_13330, KOX_13865, KOX_16945, KOX_16755, rnfD, KOX_26070 is detected, or a mutation in at least one of the positions of 5645611, 2880820, 3001328, 3678273, 3636466, 4740803, 5626010.

According to certain embodiments of the method of the twelfth and/or thirteenth aspect of the present invention, as well as also of the eighteenth aspect of the present invention, the antibiotic is a benzene derived/sulfonamide antibiotic, particularly T/S, and a mutation in at least one of the genes listed in Table 10a and/or Table 10b is detected, or a mutation in at least one of the positions (denoted POS in the tables) listed in Table 10a and/or Table 10b.

According to certain embodiments of the method of the twelfth and/or thirteenth aspect of the present invention, as well as also of the eighteenth aspect of the present invention, the Klebsiella species is particularly Klebsiella pneumonia, the antibiotic is a benzene derived/sulfonamide antibiotic, particularly T/S, and a mutation in at least one of the genes listed in Table 10a is detected, or a mutation in at least one of the positions (denoted POS in the tables) listed in Table 10a.

According to certain embodiments of the method of the twelfth and/or thirteenth aspect of the present invention, as well as also of the eighteenth aspect of the present invention, the Klebsiella species is particularly Klebsiella oxytoca, the antibiotic is a benzene derived/sulfonamide antibiotic, particularly T/S, and a mutation in at least one of the genes listed in Table 10b is detected, or a mutation in at least one of the positions (denoted POS in the tables) listed in Table 10b.

TABLE 10a
List for benzene derived/sulfonamide antibiotics, particularly for Klebsiella pneumoniae

| | | | p-value | genbank protein |
|---|---|---|---|---|
| gene name | POS | antibiotic | (FDR) | accession number |
| parC | 3763210 | CF; T/S; TE; CFT; LVX; GM; IMP; CFZ; | 1.9784E−152 | YP_001337063.1 |
| | | CRM; ETP; CP; CAX; AZT; P/T; CPE; AM; | | |
| | | A/S; CAZ; TO; MER; AUG | | |
| KPN_01607 | 1784305 | CF; T/S; TE; CFT; LVX; GM; IMP; CFZ; | 1.5316E−115 | YP_001335268.1 |
| | | CRM; ETP; CP; CAX; AZT; P/T; CPE; AM; | | |
| | | A/S; CAZ; TO; MER; AUG | | |
| KPN_01607 | 1784302 | CF; T/S; TE; CFT; LVX; GM; IMP; CFZ; | 8.1983E−115 | YP_001335268.1 |
| | | CRM; ETP; CP; CAX; AZT; P/T; CPE; AM; | | |
| | | A/S; CAZ; TO; MER; AUG | | |
| gyrA | 2905411 | CF; T/S; TE; CFT; LVX; GM; IMP; CFZ; | 4.3727E−106 | YP_001336287.1 |
| | | CRM; ETP; CP; CAX; AZT; P/T; CPE; AM; | | |
| | | A/S; CAZ; TO; MER; AUG | | |
| KPN_02451 | 2673906 | CF; T/S; TE; CFT; LVX; GM; IMP; CFZ; | 5.0133E−104 | YP_001336099.1 |
| | | CRM; ETP; CP; CAX; AZT; P/T; CPE; AM; | | |
| | | A/S; CAZ; TO; MER; AUG | | |
| baeR | 2773232 | CF; T/S; TE; CFT; LVX; GM; IMP; CFZ; | 5.5237E−104 | YP_001336179.1 |
| | | CRM; ETP; CP; CAX; AZT; P/T; CPE; AM; | | |
| | | A/S; CAZ; TO; MER; AUG | | |
| aceF | 140517 | CF; T/S; TE; CFT; LVX; GM; IMP; CFZ; | 1.3942E−103 | YP_001333809.1 |
| | | CRM; ETP; CP; CAX; AZT; P/T; CPE; AM; | | |
| | | A/S; CAZ; TO; MER; AUG | | |
| ybgH | 809148 | CF; T/S; TE; CFT; LVX; GM; IMP; CFZ; | 1.3942E−103 | YP_001334393.1 |
| | | CRM; ETP; CP; CAX; AZT; P/T; CPE; AM; | | |
| | | A/S; CAZ; TO; MER; AUG | | |
| ynjE | 1364586 | CF; T/S; TE; CFT; LVX; GM; IMP; CFZ; | 1.3942E−103 | YP_001334876.1 |
| | | CRM; ETP; CP; CAX; AZT; P/T; CPE; AM; | | |
| | | A/S; CAZ; TO; MER; AUG | | |
| KPN_01951 | 2150691 | CF; T/S; TE; CFT; LVX; GM; IMP; CFZ; | 1.3942E−103 | YP_001335612.1 |
| | | CRM; ETP; CP; CAX; AZT; P/T; CPE; AM; | | |
| | | A/S; CAZ; TO; MER; AUG | | |
| KPN_01961 | 2159024 | CF; T/S; TE; CFT; LVX; GM; IMP; CFZ; | 1.3942E−103 | YP_001335622.1 |
| | | CRM; ETP; CP; CAX; AZT; P/T; CPE; AM; | | |
| | | A/S; CAZ; TO; MER; AUG | | |
| KPN_02114 | 2317024 | CF; T/S; TE; CFT; LVX; GM; IMP; CFZ; | 1.3942E−103 | YP_001335772.1 |
| | | CRM; ETP; CP; CAX; AZT; P/T; CPE; AM; | | |
| | | A/S; CAZ; TO; MER; AUG | | |
| mhpA | 2325877 | CF; T/S; TE; CFT; LVX; GM; IMP; CFZ; | 1.3942E−103 | YP_001335780.1 |
| | | CRM; ETP; CP; CAX; AZT; P/T; CPE; AM; | | |
| | | A/S; CAZ; TO; MER; AUG | | |
| KPN_02128 | 2331649 | CF; T/S; TE; CFT; LVX; GM; IMP; CFZ; | 1.3942E−103 | YP_001335786.1 |
| | | CRM; ETP; CP; CAX; AZT; P/T; CPE; AM; | | |
| | | A/S; CAZ; TO; MER; AUG | | |
| KPN_02144 | 2347930 | CF; T/S; TE; CFT; LVX; GM; IMP; CFZ; | 1.3942E−103 | YP_001335802.1 |
| | | CRM; ETP; CP; CAX; AZT; P/T; CPE; AM; | | |
| | | A/S; CAZ; TO; MER; AUG | | |
| KPN_02149 | 2355785 | CF; T/S; TE; CFT; LVX; GM; IMP; CFZ; | 1.3942E−103 | YP_001335807.1 |
| | | CRM; ETP; CP; CAX; AZT; P/T; CPE; AM; | | |
| | | A/S; CAZ; TO; MER; AUG | | |

TABLE 10b
List for benzene derived/sulfonamide antibiotics,
particularly for Klebsiella oxytoca

| | | | p-value | genbank protein |
|---|---|---|---|---|
| gene name | POS | antibiotic | (FDR) | accession number |
| KOX_26125 | 5645611 | CF; T/S; TE; CFT; CFZ; CRM; CP; CAX; AZT; | 6.03526E−61 | YP_005021173.1 |
| | | P/T; CPE; AM; A/S; CAZ; LVX; AUG | | |
| KOX_19645 | 4247719 | CF; T/S; A/S; CRM; CAX; P/T; LVX; AM; | 7.88021E−35 | YP_005019890.1 |
| | | CFZ; AUG | | |
| KOX_18320 | 3962325 | T/S; CF; A/S; CRM; P/T; CFZ; AUG | 9.99935E−22 | YP_005019625.1 |
| KOX_02920 | 617510 | CF; T/S; CP; CFT; CFZ; CRM; AZT; P/T; | 9.66285E−19 | YP_005016564.1 |
| | | CPE; A/S; CAZ; LVX; AUG | | |
| KOX_01370 | 298246 | CF; T/S; CP; A/S; CRM; CAX; P/T; CPE; | 3.94936E−16 | YP_005016258.1 |
| | | LVX; AUG | | |
| KOX_01470 | 317306 | CF; T/S; CP; A/S; CRM; CAX; P/T; CPE; | 3.94936E−16 | YP_005016278.1 |
| | | LVX; AUG | | |
| KOX_03050 | 644166 | CF; T/S; CP; A/S; CRM; CAX; P/T; CPE; | 3.94936E−16 | YP_005016590.1 |
| | | LVX; AUG | | |
| KOX_03630 | 761146 | CF; T/S; CP; A/S; CRM; CAX; P/T; CPE; | 3.94936E−16 | YP_005016704.1 |
| | | LVX; AUG | | |
| KOX_05300 | 1090175 | CF; T/S; CP; A/S; CRM; CAX; P/T; CPE; | 3.94936E−16 | YP_005017036.1 |
| | | LVX; AUG | | |
| treF | 1093331 | CF; T/S; CP; A/S; CRM; CAX; P/T; CPE; | 3.94936E−16 | YP_005017038.1 |
| | | LVX; AUG | | |
| KOX_16020 | 3463757 | CF; T/S; CP; A/S; CRM; CAX; P/T; CPE; | 3.94936E−16 | YP_005019165.1 |
| | | LVX; AUG | | |
| KOX_16060 | 3471289 | CF; T/S; CP; A/S; CRM; CAX; P/T; CPE; | 3.94936E−16 | YP_005019173.1 |
| | | LVX; AUG | | |
| celA | 5859229 | CF; T/S; CP; A/S; CRM; CAX; P/T; CPE; | 3.94936E−16 | YP_005021375.1 |
| | | LVX; AUG | | |
| KOX_03630 | 761142 | CF; T/S; CP; A/S; CRM; CAX; P/T; CPE; | 4.13529E−16 | YP_005016704.1 |
| | | LVX; AUG | | |
| KOX_04160 | 868434 | CF; T/S; CP; A/S; CRM; CAX; P/T; CPE; | 4.13529E−16 | YP_005016808.1 |
| | | LVX; AUG | | |

A fourteenth aspect of the present invention is directed to a diagnostic method of determining an infection of a patient with Klebsiella species, particularly Klebsiella pneumoniae and/or Klebsiella oxytoca, potentially resistant to antimicrobial drug treatment, which can also be described as method of determining an antimicrobial drug, e.g. antibiotic, resistant Klebsiella, particularly Klebsiella pneumoniae and/or Klebsiella oxytoca, infection of a patient, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least one gene from the group of genes consisting of KPN_01607, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, preferably from the group of genes consisting of KPN_01607, KPN_02451, ybgH, ynjE, KPN_01951, KPN_01961, KPN_02114, mhpA, KPN_02128, KPN_02144, KPN_02149, ydiJ, btuE, oppC, pth, KPN_02298, KPN_02302, cbl, hisB, yegQ, yehY, KPN_02580, KPN_02621, yfaW, KPN_02170, KPN_02025, livG, livM, livH, fliY, yedQ, abgB, treA, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, wherein the presence of said at least one mutation is indicative of an antimicrobial drug, e.g. antibiotic, resistant Klebsiella, particularly Klebsiella pneumoniae and/or Klebsiella oxytoca, infection in said patient.

According to certain embodiments, the fourteenth aspect relates to a diagnostic method of determining an infection of a patient with Klebsiella species, particularly Klebsiella pneumoniae, potentially resistant to antimicrobial drug treatment, which can also be described as method of determining an antimicrobial drug, e.g. antibiotic, resistant Klebsiella, particularly Klebsiella pneumoniae, infection of a patient, comprising the steps of:

a) obtaining or providing a sample containing or suspected of containing at least one Klebsiella species, particularly Klebsiella pneumoniae, from the patient;
b) determining the presence of at least one mutation in at least one gene from the group of genes consisting of KPN_01607, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, preferably from the group of genes consisting of KPN_01607, KPN_02451, ybgH, ynjE, KPN_01951, KPN_01961, KPN_02114, mhpA, KPN_02128, KPN_02144, KPN_02149, ydiJ, btuE, oppC, pth, KPN_02298, KPN_02302, cbl, hisB, yegQ, yehY, KPN_02580, KPN_02621, yfaW, KPN_02170, KPN_02025, livG, livM, livH, fliY, yedQ, abgB, treA, KPN_02399, ydcR, anmK, ccmF, KPN_02440, KPN_02540, KPN_01752, and KPN_04195, wherein the presence of said at least one mutation is indicative of an antimicrobial drug, e.g. antibiotic, resistant Klebsiella, particularly Klebsiella pneumonia, infection in said patient.

According to certain embodiments, the fourteenth aspect relates to a diagnostic method of determining an infection of a patient with Klebsiella species, particularly Klebsiella oxytoca, potentially resistant to antimicrobial drug treatment, which can also be described as method of determining an antimicrobial drug, e.g. antibiotic, resistant Klebsiella, particularly Klebsiella oxytoca, infection of a patient, comprising the steps of:

a) obtaining or providing a sample containing or suspected of containing at least one Klebsiella species, particularly Klebsiella oxytoca, from the patient;
b) determining the presence of at least one mutation in at least one gene from the group of genes consisting of KOX_26125, KOX_13365, KOX_16735, KOX_25695, KOX_12270, and KOX_15055, wherein the presence of said at least one mutation is indicative of an antimicrobial drug, e.g. antibiotic, resistant Klebsiella, particularly Klebsiella oxytoca, infection in said patient.

A fifteenth aspect of the present invention is directed to a method of selecting a treatment of a patient suffering from an antimicrobial drug, e.g. antibiotic, resistant Klebsiella, particularly Klebsiella pneumoniae and/or Klebsiella oxytoca, infection, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least one gene from the group of genes consisting of KPN_01607, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, preferably from the group of genes consisting of KPN_01607, KPN_02451, ybgH, ynjE, KPN_01951, KPN_01961, KPN_02114, mhpA, KPN_02128, KPN_02144, KPN_02149, ydiJ, btuE, oppC, pth, KPN_02298, KPN_02302, cbl, hisB, yegQ, yehY, KPN_02580, KPN_02621, yfaW, KPN_02170, KPN_02025, livG, livM, livH, fliY, yedQ, abgB, treA, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, wherein the presence of said at least one mutation is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection.

According to certain embodiments, the fifteenth aspect relates to a method of selecting a treatment of a patient suffering from an antimicrobial drug, e.g. antibiotic, resistant Klebsiella, particularly Klebsiella pneumonia, infection, comprising the steps of:

a) obtaining or providing a sample containing or suspected of containing at least one Klebsiella species, particularly Klebsiella pneumoniae, from the patient;
b) determining the presence of at least one mutation in at least one gene from the group of genes consisting of KPN_01607, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, preferably from the group of genes consisting of KPN_01607, KPN_02451, ybgH, ynjE, KPN_01951, KPN_01961, KPN_02114, mhpA, KPN_02128, KPN_02144, KPN_02149, ydiJ, btuE, oppC, pth, KPN_02298, KPN_02302, cbl, hisB, yegQ, yehY, KPN_02580, KPN_02621, yfaW, KPN_02170, KPN_02025, livG, livM, livH, fliY, yedQ, abgB, treA, KPN_02399, ydcR, anmK, ccmF, KPN_02440, KPN_02540, KPN_01752, and KPN_04195, wherein the presence of said at least one mutation is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae, infection.

According to certain embodiments, the fifteenth aspect relates to a method of selecting a treatment of a patient suffering from an antimicrobial drug, e.g. antibiotic, resistant Klebsiella, particularly Klebsiella oxytoca, infection, comprising the steps of:

a) obtaining or providing a sample containing or suspected of containing at least one Klebsiella species, particularly Klebsiella oxytoca, from the patient;
b) determining the presence of at least one mutation in at least one gene from the group of genes consisting of KOX_26125, KOX_13365, KOX_16735, KOX_25695, KOX_12270, and KOX_15055, wherein the presence of said at least one mutation is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae, infection.

Again, in the fourteenth and the fifteenth aspect the steps correspond to those in the first or second aspect, although only a mutation in at least one gene is determined.

A sixteenth aspect of the present invention is directed to a method of treating a patient suffering from an antimicrobial drug, e.g. antibiotic, resistant Klebsiella, particularly Klebsiella pneumoniae and/or Klebsiella oxytoca, infection, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least one gene from the group of genes consisting of KPN_01607, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, preferably from the group of genes consisting of KPN_01607, KPN_02451, ybgH, ynjE, KPN_01951, KPN_01961, KPN_02114, mhpA, KPN_02128, KPN_02144, KPN_02149, ydiJ, btuE, oppC, pth, KPN_02298, KPN_02302, cbl, hisB, yegQ, yehY, KPN_02580, KPN_02621, yfaW, KPN_02170, KPN_02025, livG, livM, livH, fliY, yedQ, abgB, treA, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, wherein the presence of said at least one mutation is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection; and
e) treating the patient with said one or more antimicrobial, e.g. antibiotic, drugs.

According to certain embodiments, the sixteenth aspect relates to a method of treating a patient suffering from an antimicrobial drug, e.g. antibiotic, resistant Klebsiella, particularly Klebsiella pneumoniae, infection, comprising the steps of:

a) obtaining or providing a sample containing or suspected of containing at least one Klebsiella species, particularly Klebsiella pneumoniae, from the patient;
b) determining the presence of at least one mutation in at least one gene from the group of genes consisting of KPN_01607, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, preferably from the group of genes consisting of KPN_01607, KPN_02451, ybgH, ynjE, KPN_01951, KPN_01961, KPN_02114, mhpA, KPN_02128, KPN_02144, KPN_02149, ydiJ, btuE, oppC, pth, KPN_02298, KPN_02302, cbl, hisB, yegQ, yehY, KPN_02580, KPN_02621, yfaW, KPN_02170, KPN_02025, livG, livM, livH, fliY, yedQ, abgB, treA, KPN_02399, ydcR, anmK, ccmF, KPN_02440, KPN_02540, KPN_01752, and KPN_04195, wherein the presence of said at least one mutation is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella pneumoniae, infection; and
e) treating the patient with said one or more antimicrobial, e.g. antibiotic, drugs.

According to certain embodiments, the sixteenth aspect relates to a method of treating a patient suffering from an antimicrobial drug, e.g. antibiotic, resistant Klebsiella, particularly Klebsiella oxytoca, infection, comprising the steps of:

a) obtaining or providing a sample containing or suspected of containing at least one Klebsiella species, particularly Klebsiella oxytoca, from the patient;
b) determining the presence of at least one mutation in at least one gene from the group of genes consisting of KOX_26125, KOX_13365, KOX_16735, KOX_25695, KOX_12270, and KOX_15055, wherein the presence of said at least one mutation is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella oxytoca, infection; and
e) treating the patient with said one or more antimicrobial, e.g. antibiotic, drugs.

A seventeenth aspect of the present invention is directed to a method of treating a patient suffering from an antimicrobial drug, e.g. antibiotic, resistant Klebsiella, particularly Klebsiella pneumoniae and/or Klebsiella oxytoca, infection, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least two genes from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, wherein the presence of said at least two mutations is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection; and
e) treating the patient with said one or more antimicrobial, e.g. antibiotic, drugs.

According to certain embodiments, the seventeenth aspect relates to a method of treating a patient suffering from an antimicrobial drug, e.g. antibiotic, resistant Klebsiella, particularly Klebsiella pneumoniae, infection, comprising the steps of:

a) obtaining or providing a sample containing or suspected of containing at least one Klebsiella species, particularly Klebsiella pneumoniae, from the patient;
b) determining the presence of at least one mutation in at least two genes from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, wherein the presence of said at least two mutations is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly

Klebsiella pneumoniae, infection; and

e) treating the patient with said one or more antimicrobial, e.g. antibiotic, drugs.

According to certain embodiments, the seventeenth aspect relates to a method of treating a patient suffering from an antimicrobial drug, e.g. antibiotic, resistant Klebsiella, particularly Klebsiella oxytoca, infection, comprising the steps of:

a) obtaining or providing a sample containing or suspected of containing at least one Klebsiella species, particularly Klebsiella oxytoca, from the patient;
b) determining the presence of at least one mutation in at least two genes from the group of genes consisting of KOX_26125, KOX_13365, KOX_16735, KOX_25695, KOX_12270, and KOX_15055, wherein the presence of said at least two mutations is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella oxytoca, infection; and
e) treating the patient with said one or more antimicrobial, e.g. antibiotic, drugs.

An eighteenth aspect of the present invention is directed to a method of treating a patient suffering from an antimicrobial drug, e.g. antibiotic, resistant Klebsiella, particularly Klebsiella pneumoniae and/or Klebsiella oxytoca, infection, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least two genes from the group of genes listed in Table 5a and/or Table 5b, wherein the presence of said at least two mutations is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection; and
e) treating the patient with said one or more antimicrobial, e.g. antibiotic, drugs.

According to certain embodiments, the eighteenth aspect relates to a method of treating a patient suffering from an antimicrobial drug, e.g. antibiotic, resistant Klebsiella, particularly Klebsiella pneumoniae, infection, comprising the steps of:

a) obtaining or providing a sample containing or suspected of containing at least one Klebsiella species, particularly Klebsiella pneumoniae, from the patient;
b) determining the presence of at least one mutation in at least two genes from the group of genes listed in Table 5a, wherein the presence of said at least two mutations is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella pneumoniae, infection; and
e) treating the patient with said one or more antimicrobial, e.g. antibiotic, drugs.

According to certain embodiments, the eighteenth aspect relates to a method of treating a patient suffering from an antimicrobial drug, e.g. antibiotic, resistant Klebsiella, particularly Klebsiella oxytoca, infection, comprising the steps of:

a) obtaining or providing a sample containing or suspected of containing at least one Klebsiella species, particularly Klebsiella oxytoca, from the patient;
b) determining the presence of at least one mutation in at least two genes from the group of genes listed in Table 5b, wherein the presence of said at least two mutations is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella oxytoca, infection; and
e) treating the patient with said one or more antimicrobial, e.g. antibiotic, drugs.

A nineteenth aspect of the present invention is directed to a method of treating a patient suffering from an antimicrobial drug, e.g. antibiotic, resistant Klebsiella, particularly Klebsiella pneumoniae and/or Klebsiella oxytoca, infection, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least one gene from the group of genes listed in Table 11a and/or Table 5b, preferably from the group of genes listed in Table 12a and/or Table 12b, wherein the presence of said at least one mutation is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection; and
e) treating the patient with said one or more antimicrobial, e.g. antibiotic, drugs.

According to certain embodiments, the nineteenth aspect relates to a method of treating a patient suffering from an antimicrobial drug, e.g. antibiotic, resistant Klebsiella, particularly Klebsiella pneumoniae, infection, comprising the steps of:

a) obtaining or providing a sample containing or suspected of containing at least one Klebsiella species, particularly Klebsiella pneumoniae, from the patient;
b) determining the presence of at least one mutation in at least one gene from the group of genes listed in Table 11a, preferably from the group of genes listed in Table 12a, wherein the presence of said at least one mutation is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella pneumoniae, infection; and
e) treating the patient with said one or more antimicrobial, e.g. antibiotic, drugs.

According to certain embodiments, the nineteenth aspect relates to a method of treating a patient suffering from an antimicrobial drug, e.g. antibiotic, resistant Klebsiella, particularly Klebsiella oxytoca, infection, comprising the steps of:

a) obtaining or providing a sample containing or suspected of containing at least one Klebsiella species, particularly Klebsiella oxytoca, from the patient;
b) determining the presence of at least one mutation in at least one gene from the group of genes listed in Table 5b, preferably from the group of genes listed in Table 12b, wherein the presence of said at least one mutation is indicative of a resistance to one or more antimicrobial, e.g. antibiotic, drugs;
c) identifying said at least one or more antimicrobial, e.g. antibiotic, drugs;
d) selecting one or more antimicrobial, e.g. antibiotic, drugs different from the ones identified in step c) and being suitable for the treatment of a Klebsiella, particularly Klebsiella oxytoca, infection; and
e) treating the patient with said one or more antimicrobial, e.g. antibiotic, drugs.

Also in the sixteenth to nineteenth aspect of the invention, steps a) to d) are analogous to the steps in the method of the second aspect of the present invention. Step e) can be sufficiently carried out without being restricted and can be done e.g. non-invasively.

A twentieth aspect of the present invention is directed to a diagnostic method of determining an infection of a patient with Klebsiella species, particularly Klebsiella pneumoniae and/or Klebsiella oxytoca, potentially resistant to antimicrobial drug treatment, which can also be described as method of determining an antimicrobial drug, e.g. antibiotic, resistant Klebsiella, particularly Klebsiella pneumoniae and/or Klebsiella oxytoca, infection of a patient, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least one gene from the group of genes listed in Table 11a and/or Table 5b, preferably from the group of genes listed in Table 12a and/or Table 12b, wherein the presence of said at least one mutation is indicative of an antimicrobial drug, e.g. antibiotic, resistant Klebsiella, particularly Klebsiella pneumoniae and/or Klebsiella oxytoca, infection in said patient.

According to certain embodiments, the twentieth aspect relates to a diagnostic method of determining an infection of a patient with Klebsiella species, particularly Klebsiella pneumoniae, potentially resistant to antimicrobial drug treatment, which can also be described as method of determining an antimicrobial drug, e.g. antibiotic, resistant Klebsiella, particularly Klebsiella pneumoniae, infection of a patient, comprising the steps of:

a) obtaining or providing a sample containing or suspected of containing at least one Klebsiella species, particularly Klebsiella pneumoniae, from the patient;
b) determining the presence of at least one mutation in at least one gene from the group of genes listed in Table 11a, preferably from the group of genes listed in Table 12a, wherein the presence of said at least one mutation is indicative of an antimicrobial drug, e.g. antibiotic, resistant Klebsiella, particularly Klebsiella pneumoniae, infection in said patient.

According to certain embodiments, the twentieth aspect relates to a diagnostic method of determining an infection of a patient with Klebsiella species, particularly Klebsiella oxytoca, potentially resistant to antimicrobial drug treatment, which can also be described as method of determining an antimicrobial drug, e.g. antibiotic, resistant Klebsiella, particularly Klebsiella oxytoca, infection of a patient, comprising the steps of:

a) obtaining or providing a sample containing or suspected of containing at least one Klebsiella species, particularly Klebsiella oxytoca, from the patient;
b) determining the presence of at least one mutation in at least one gene from the group of genes listed in Table 5b, preferably from the group of genes listed in Table 12b, wherein the presence of said at least one mutation is indicative of an antimicrobial drug, e.g. antibiotic, resistant Klebsiella, particularly Klebsiella oxytoca, infection in said patient.

A twenty-first aspect of the present invention is directed to a method of selecting a treatment of a patient suffering from an antimicrobial drug, e.g. antibiotic, resistant Klebsiella, particularly Klebsiella pneumoniae and/or Klebsiella oxytoca, infection, comprising the steps of:

a) obtaining or providing a sample containing or suspected of containing at least one Klebsiella species, particularly Klebsiella pneumoniae and/or Klebsiella oxytoca, from the patient;
b) determining the presence of at least one mutation in at least one gene from the group of genes listed in Table 11a and/or Table 5b, preferably from the group of genes listed in Table 12a and/or Table 12b, wherein the presence of said at least one mutation is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae and/or Klebsiella oxytoca, infection.

According to certain embodiments, the twenty-first aspect relates to a method of selecting a treatment of a patient suffering from an antimicrobial drug, e.g. antibiotic, resistant Klebsiella, particularly Klebsiella pneumoniae, infection, comprising the steps of:

a) obtaining or providing a sample containing or suspected of containing at least one Klebsiella species, particularly Klebsiella pneumoniae, from the patient;
b) determining the presence of at least one mutation in at least one gene from the group of genes listed in Table 11a, preferably from the group of genes listed in Table 12a, wherein the presence of said at least one mutation is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella pneumoniae, infection.

According to certain embodiments, the twenty-first aspect relates to a method of selecting a treatment of a patient suffering from an antimicrobial drug, e.g. antibiotic, resistant Klebsiella, particularly Klebsiella oxytoca, infection, comprising the steps of:

a) obtaining or providing a sample containing or suspected of containing at least one Klebsiella species, particularly Klebsiella oxytoca, from the patient;
b) determining the presence of at least one mutation in at least one gene from the group of genes listed in Table 5b, preferably from the group of genes listed in Table 12b, wherein the presence of said at least one mutation is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella, particularly Klebsiella oxytoca, infection.

Again, in the twentieth and the twenty-first aspect the steps correspond to those in the first or second aspect, although only a mutation in at least one gene is determined.

TABLE 11a
List of genes, particularly for Klebsiella pneumoniae

| | | | | |
|---|---|---|---|---|
| KPN_01752 | KPN_01607 | KPN_04195 | KPN_02451 | baeR |
| aceF | ybgH | ynjE | KPN_01951 | KPN_01961 |
| KPN_02114 | mhpA | KPN_02128 | KPN_02144 | KPN_02149 |
| ydiJ | btuE | oppC | pth | KPN_02298 |
| KPN_02302 | dadA | yoaA | ftn | cbl |
| hisB | yegQ | yehY | KPN_02580 | yejH |
| KPN_02621 | yfaW | KPN_02170 | KPN_02025 | livG |
| livM | livH | fliY | yedQ | abgB |
| treA | baeS | KPN_02399 | ydcR | anmK |
| ccmF | KPN_02440 | KPN_02540 | | |

TABLE 12a
List of genes, particularly for Klebsiella pneumoniae

| | | | | |
|---|---|---|---|---|
| KPN_01752 | KPN_01607 | KPN_04195 | KPN_02451 | KPN_02540 |
| KPN_02440 | ybgH | ynjE | KPN_01951 | KPN_01961 |
| KPN_02114 | mhpA | KPN_02128 | KPN_02144 | KPN_02149 |
| ydiJ | btuE | oppC | pth | KPN_02298 |
| KPN_02302 | ccmF | anmK | ydcR | cbl |
| hisB | yegQ | yehY | KPN_02580 | KPN_02399 |
| KPN_02621 | yfaW | KPN_02170 | KPN_02025 | livG |
| livM | livH | fliY | yedQ | abgB |
| treA | | | | |

In a twenty-second aspect the present invention relates to a method of determining an antibiotic resistance profile for a bacterial microorganism belonging to the species E. coli and/or Klebsiella pneumoniae comprising the steps of
providing a sample containing or suspected of containing the bacterial microorganism belonging to the species E. coli;
determining the presence of a mutation in at least one gene selected from the group of genes described below, particularly with regard to Examples 2 and 3 and/or selected from the group of mutations described below, particularly with regard to Examples 2 and 3;
   wherein the presence of a mutation is indicative of a resistance to an antibiotic drug.

TABLE 12b
List of genes, particularly for Klebsiella oxytoca

| | | | | | |
|---|---|---|---|---|---|
| KOX_26125 | KOX_13365 | KOX_16735 | KOX_25695 | KOX_12270 | KOX_15055 |
| KOX_02920 | KOX_13330 | KOX_09205 | KOX_19645 | KOX_23415 | KOX_16785 |
| KOX_04215 | KOX_05500 | malS | KOX_06515 | KOX_14735 | KOX_15150 |
| KOX_18350 | KOX_26135 | zntB | KOX_07410 | KOX_00765 | metH |
| KOX_25845 | KOX_23215 | KOX_23670 | KOX_07500 | KOX_12235 | KOX_10070 |
| KOX_01110 | KOX_01370 | KOX_13865 | KOX_16945 | KOX_16755 | rnfD |
| KOX_26070 | KOX_18320 | KOX_01470 | KOX_03050 | KOX_03630 | KOX_05300 |
| treF | KOX_16020 | KOX_16060 | celA | KOX_04160 | |

In a twenty-third aspect the present invention relates to a method of determining the resistance of a bacterial microorganism belonging to the species E. coli and/or Klebsiella pneumoniae to an antibiotic drug comprising:
providing a sample containing or suspected of containing the bacterial microorganism belonging to the species E. coli;
determining from said sample a nucleic acid sequence information of at least one gene selected from the group of genes described below, particularly with regard to Examples 2 and 3; and
based on the determination of said genetic information determining the resistance to the antibiotic drug.

According to certain embodiments of the twenty-second and twenty-third aspect, the present invention relates to at least one of the methods of the twenty-second and twenty-third aspect, wherein determining the nucleic acid sequence information or the presence of a mutation comprises determining the presence of a single nucleotide at a single position in at least one gene, in particular a mutation as described hereinabove, in particular a mutation leading to at least one alteration of an amino acid sequence encoded by the nucleic acid sequence.

According to certain embodiments of the twenty-second and twenty-third aspect, the present invention relates to at least one of the methods of the twenty-second and twenty-third aspect, wherein the presence of a single nucleotide polymorphism or mutation at a single nucleotide position is detected in at least one gene selected from the group of genes described hereinabove.

According to certain embodiments of the twenty-second and twenty-third aspect, the present invention relates to at least one of the methods of the twenty-second and twenty-third aspect, wherein a detected mutation is a mutation leading to an altered amino acid sequence in a polypeptide derived from a respective gene in which the detected mutation is located.

According to certain embodiments of the twenty-second and twenty-third aspect, the present invention relates to at least one of the methods of the twenty-second and twenty-third aspect, wherein the mutation is a mutation which is selected from the group of mutations described hereinabove.

According to certain embodiments of the twenty-second and twenty-third aspect, the present invention relates to at least one of the methods of the twenty-second and twenty-third aspect, wherein determining the nucleic acid sequence information or the presence of a mutation comprises determining a partial sequence or an entire sequence of the at least one gene.

According to certain embodiments of the twenty-second and twenty-third aspect, the present invention relates to at least one of the methods of the twenty-second and twenty-third aspect, wherein determining the nucleic acid sequence information or the presence of a mutation comprises determining a partial or entire sequence of the genome of said bacterial microorganism, wherein said partial or entire sequence of the genome comprises at least a partial sequence of said at least one gene.

According to certain embodiments of the twenty-second and twenty-third aspect, the present invention relates to at least one of the methods of the twenty-second and twenty-third aspect, wherein the sample is a patient sample (clinical isolate).

According to certain embodiments of the twenty-second and twenty-third aspect, the present invention relates to at least one of the methods of the twenty-second and twenty-third aspect, wherein determining the nucleic acid sequence information or the presence of a mutation comprises a using a next generation sequencing or high throughput sequencing method.

According to certain embodiments of the twenty-second and twenty-third aspect, the present invention relates to at least one of the methods of the twenty-second and twenty-third aspect, wherein a partial or entire genome sequence of the bacterial organism is determined by a using a next generation sequencing or high throughput sequencing method.

According to certain embodiments of the twenty-second and twenty-third aspect, the present invention relates to at least one of the methods of the twenty-second and twenty-third aspect, wherein determining the nucleic acid sequence information or the presence of a mutation comprises determining a nucleic acid sequence information or mutation of 2, 3, 4, 5, 6, 7, 8 or 9 genes selected from the group genes described hereinabove.

According to certain embodiments of the twenty-second and twenty-third aspect, the present invention relates to at least one of the methods of the twenty-second and twenty-third aspect, wherein the method of the invention further comprises determining the resistance to 2, 3, 4, 5, or 6 antibiotic drugs.

### EXAMPLES

The present invention will now be described in detail with reference to several examples thereof. However, these examples are illustrative and do not limit the scope of the invention.

### Example 1

Whole genome sequencing was carried out in addition to classical antimicrobial susceptibility testing of the same isolates for a cohort of 1576 specimens, particularly 1176 for Klebsiella pneumonia and 400 for Klebsiella oxytoca. This allowed performing genome wide correlation studies to find genetic variants (e.g. point mutations, small insertions and deletion, larger structural variants, plasmid copy number gains, gene dosage effects) in the genome and plasmids that are significantly correlated to the resistance against one or several drugs. The approach also allows for comparing the relevant sites in the genome to each other.

In the approach the different sources of genetic resistance as well as the different ways of how bacteria can become resistant were covered. By measuring clinical isolates collected in a broad geographical area and across a broad time span of three decades a complete picture going far beyond the rather artificial step of laboratory generated resistance mechanisms was tried to be generated.

To this end, a set of 21 clinically relevant antimicrobial agents with 5 different modes of action was put together, and the minimally inhibitory concentration (MIC) of the 21 drugs for the Klebsiella isolates was measured.

The detailed procedure is given in the following:

### Bacterial Strains

The inventors selected 1576 Klebsiella strains, particularly 1176 for Klebsiella pneumonia and 400 for Klebsiella oxytoca, from the microbiology strain collection at Siemens Healthcare Diagnostics (West Sacramento, Calif.) for susceptibility testing and whole genome sequencing.

Antimicrobial Susceptibility Testing (AST) Panels Frozen reference AST panels were prepared following Clinical Laboratory Standards Institute (CLSI) recommendations. The following antimicrobial agents (with μg/ml concentrations shown in parentheses) were included in the panels: Amoxicillin/K Clavulanate (0.5/0.25-64/32), Ampicillin (0.25-128), Ampicillin/Sulbactam (0.5/0.25-64/32), Aztreonam (0.25-64), Cefazolin (0.5-32), Cefepime (0.25-64), Cefotaxime (0.25-128), Ceftazidime (0.25-64), Ceftriaxone (0.25-128), Cefuroxime (1-64), Cephalothin (1-64), Ciprofloxacin (0.015-8), Ertepenem (0.12-32), Gentamicin (0.12-32), Imipenem (0.25-32), Levofloxacin (0.25-16), Meropenem (0.12-32), Piperacillin/Tazobactam (0.25/4-256/4), Tetracycline (0.5-64), Tobramycin (0.12-32), and Trimethoprim/Sulfamethoxazole (0.25/4.7-32/608). Prior to use with clinical isolates, AST panels were tested with QC strains. AST panels were considered acceptable for testing with clinical isolates when the QC results met QC ranges described by CLSI16.

### Inoculum Preparation

Isolates were cultured on trypticase soy agar with 5% sheep blood (BBL, Cockeysville, Md.) and incubated in ambient air at 35±1° C. for 18-24 h. Isolated colonies (4-5 large colonies or 5-10 small colonies) were transferred to a 3 ml Sterile Inoculum Water (Siemens) and emulsified to a final turbidity of a 0.5 McFarland standard. 2 ml of this suspension was added to 25 ml Inoculum Water with Pluronic-F (Siemens). Using the Inoculator (Siemens) specific for frozen AST panels, 5 μl of the cell suspension was transferred to each well of the AST panel. The inoculated AST panels were incubated in ambient air at 35±1° C. for 16-20 h. Panel results were read visually, and minimal inhibitory concentrations (MIC) were determined.

### DNA Extraction

Four streaks of each Gram-negative bacterial isolate cultured on trypticase soy agar containing 5% sheep blood and cell suspensions were made in sterile 1.5 ml collection tubes containing 50 μl Nuclease-Free Water (AM9930, Life Technologies). Bacterial isolate samples were stored at −20° C. until nucleic acid extraction. The Tissue Preparation System (TPS) (096D0382-02_01_B, Siemens) and the VERSANT® Tissue Preparation Reagents (TPR) kit (10632404B, Siemens) were used to extract DNA from these bacterial isolates. Prior to extraction, the bacterial isolates were thawed at room temperature and were pelleted at 2000 G for 5 seconds. The DNA extraction protocol DNAext was used for complete total nucleic acid extraction of 48 isolate samples and eluates, 50 μl each, in 4 hours. The total nucleic acid eluates were then transferred into 96-Well qPCR Detection Plates (401341, Agilent Technologies) for RNase A digestion, DNA quantitation, and plate DNA concentration standardization processes. RNase A (AM2271, Life Technologies) which was diluted in nuclease-free water following manufacturer's instructions was added to 50 μl of the total nucleic acid eluate for a final working concentration of 20 μg/ml. Digestion enzyme and eluate mixture were incubated at 37° C. for 30 minutes using Siemens VERSANT® Amplification and Detection instrument. DNA from the RNase digested eluate was quantitated using the Quant-iT™ PicoGreen dsDNA Assay (P11496, Life Technologies) following the assay kit instruction, and fluorescence was determined on the Siemens VERSANT® Amplification and Detection instrument. Data analysis was performed using Microsoft® Excel 2007. 25 μl of the quantitated DNA eluates were transferred into a new 96-Well PCR plate for plate DNA concentration standardization prior to library preparation. Elution buffer from the TPR kit was used to adjust DNA concentration. The standardized DNA eluate plate was then stored at −80° C. until library preparation.

### Next Generation Sequencing

Prior to library preparation, quality control of isolated bacterial DNA was conducted using a Qubit 2.0 Fluorometer (Qubit dsDNA BR Assay Kit, Life Technologies) and an Agilent 2200 TapeStation (Genomic DNA ScreenTape, Agilent Technologies). NGS libraries were prepared in 96 well format using NexteraXT DNA Sample Preparation Kit and NexteraXT Index Kit for 96 Indexes (Illumina) according to the manufacturer's protocol. The resulting sequencing libraries were quantified in a qPCR-based approach using the KAPA SYBR FAST qPCR MasterMix Kit (Peqlab) on a ViiA 7 real time PCR system (Life Technologies). 96 samples were pooled per lane for paired-end sequencing (2×100 bp) on Illumina Hiseq2000 or Hiseq2500 sequencers using TruSeq PE Cluster v3 and TruSeq SBS v3 sequncing chemistry (Illumina). Basic sequencing quality parameters were determined using the FastQC quality control tool for high throughput sequence data (Babraham Bioinformatics Institute).

### Data Analysis

Raw paired-end sequencing data for the 1576 Klebsiella samples, particularly 1176 for Klebsiella pneumonia and 400 for Klebsiella oxytoca, were mapped against the Klebsiella reference (NC_009648 for Klebsiella pneumonia, NC_016612 for Klebsiella oxytoca) with BWA 0.6.1.20. The resulting SAM files were sorted, converted to BAM files, and PCR duplicates were marked using the Picard tools package 1.104 (http://picard.sourceforge.net/). The Genome Analysis Toolkit 3.1.1 (GATK) was used to call SNPs and indels for blocks of 200 Klebsiella samples (parameters: -ploidy 1 -glm BOTH -stand_call_conf 30 -stand_emit_conf 10). VCF files were combined into a single file and quality filtering for SNPs was carried out (QD<2.0∥FS>60.0∥MQ<40.0) and indels (QD<2.0∥FS>200.0). Detected variants were annotated with SnpEff22 to predict coding effects. For each annotated position, genotypes of all Klebsiella samples were considered. Klebsiella samples were split into two groups, low resistance group (having lower MIC concentration for the considered drug), and high resistance group (having higher MIC concentrations) with respect to a certain MIC concentration (breakpoint). To find the best breakpoint all thresholds were evaluated and p-values were computed with Fisher's exact test relying on a 2×2 contingency table (number of Klebsiella samples having the reference or variant genotype vs. number of samples belonging to the low and high resistance group). The best computed breakpoint was the threshold yielding the lowest p-value for a certain genomic position and drug. For further analyses positions with non-synonymous alterations and p-value <10⁻¹¹ were considered. Based on the contingency table, the accuracy (ACC), sensitivity (SENS), specificity (SPEC), and the positive/negative predictive values (PPV/NPV) were calculated.

Since a potential reason for drug resistance is gene duplication, gene dose dependency was evaluated. For each sample the genomic coverage for each position was determined using BED Tools. Gene ranges were extracted from the reference assemblies NC_009648.gff and NC_016612.gff and the normalized median coverage per gene was calculated. To compare low- and high-resistance isolates the best area under the curve (AUC) value was computed. Groups of at least 20% of all samples having a median coverage larger than zero for that gene and containing more than 15 samples per group were considered in order to exclude artifacts and cases with AUC>0.75 were further evaluated.

To include data on the different ways how resistance mechanisms are acquired Klebsiella isolates collected over more than three decades were analyzed such that also horizontal gene transfer could potentially be discovered.

In detail, the following steps were carried out: Klebsiella strains to be tested were seeded on agar plates and incubated under growth conditions for 24 hours. Then, colonies were picked and incubated in growth medium in the presence of a given antibiotic drug in dilution series under growth conditions for 16-20 hours. Bacterial growth was determined by observing turbidity.

Next mutations were searched that are highly correlated with the results of the phenotypic resistance test.

For sequencing, samples were prepared using a Nextera library preparation, followed by multiplexed sequencing using the Illuminat HiSeq 2500 system, paired end sequencing. Data were mapped with BWA (Li H. and Durbin R. (2010) Fast and accurate long-read alignment with Burrows-Wheeler Transform. Bioinformatics, Epub. [PMID: 20080505]) and SNP were called using samtools (Li H.*, Handsaker B.*, Wysoker A., Fennell T., Ruan J., Homer N., Marth G., Abecasis G., Durbin R. and 1000 Genome Project Data Processing Subgroup (2009) The Sequence alignment/map (SAM) format and SAMtools. Bioinformatics, 25, 2078-9. [PMID: 19505943]).

As reference genomes, NC_009648 for Klebsiella pneumonia and NC_016612 for Klebsiella oxytoca, as annotated at the NCBI, was determined as best suited.

The mutations were matched to the genes and the amino acid changes were calculated. Using different algorithms (SVM, homology modeling) mutations leading to amino acid changes with likely pathogenicity/resistance were calculated.

In total, whole genomes and plasmids of 1576 different clinical isolates of Klebsiella species, particularly 1176 for Klebsiella pneumonia and 400 for Klebsiella oxytoca, were sequenced, and classical antimicrobial susceptibility testing (AST) against 21 therapy forms as described above was performed for all organisms. From the classical AST two tables with 1176, respectively 400 rows (isolates) and 21 columns (MIC values for 21 drugs) was obtained. Each table entry contained the MIC for the respective isolate and the respective drug. The genetic data were mapped to different reference genomes of Klebsiella that have been annotated at the NCBI (http://www.ncbi.nlm.nih.gov/), and the best reference was chosen as template for the alignment—NC_009648 for Klebsiella pneumonia and NC_016612 for Klebsiella oxytoca as annotated at the NCBI. Additionally, assemblies were carried out and it was verified that the sequenced genomes fulfil all quality criteria to become reference genomes.

Next, genetic variants were evaluated. This approach resulted in a table with the genetic sites in columns and the same isolates in 1176, respectively 400 rows. Each table entry contained the genetic determinant at the respective site (A, C, T, G, small insertions and deletions, . . . ) for the respective isolate.

In a next step different statistical tests were carried out
1) For comparing resistance/susceptibility to genetic sites we calculated contingency tables and determined the significance using Fishers test
2) For comparing different sites to each other we calculated the correlation between different genetic sites
3) For detecting gene dosage effects, e.g. loss or gain of genes (in the genome or on plasmids) we calculated the coverage (i.e. how many read map to the current position) at each site for resistant and not resistant isolates.

From the data, first the genes with the best p-value were chosen for the list of mutations as well as the list of correlated antibiotic resistance, representing Tables 1a and 1b and Tables 2a and 2b, respectively.

A full list of all genetic sites, drugs, drug classes, affected genes etc. is provided in Tables 3a and 3b and 4a, 4b, 4c, 4d, 4e, and 4f, wherein Table 3a corresponds to Table 1a (for Klebsiella pneumoniae) and Table 3b corresponds to Table 1b (for Klebsiella oxytoca), and they represent the genes having the lowest p-values after determining mutations in the genes. Tables 4a, 4b and 4c (for Klebsiella pneumoniae) and Tables 4d, 4e, and 4f (for Klebsiella oxytoca), respectively, correspond to Tables 2a and 2b, respectively, and represent the genes having the lowest p-values after correlating the mutations with antibiotic resistance for the respective antibiotics.

In addition, the data with the best p-values for each antibiotic class with the most antibiotic drugs as well as each antibiotic, respectively, were evaluated, being disclosed in Tables 5a, 5b, 6a, 6b, 7a, 7b, 8, 9a, 9b, 10a and 10b.

In Tables 3-10b the columns are designated as follows:

Gene name: affected gene;
POS: genomic position of the SNP/variant in the Enterobacter reference genome (see above);
p-value: significance value calculated using Fishers exact test (determined according to FDR (Benjamini Hochberg) method (Benjamini Hochberg, 1995));
genbank protein accession number: (NCBI) Accession number of the corresponding protein of the genes

TABLE 3a
Detailed results for the genes in Example 1 for Klebsiella pneumoniae
(corresponding to Table 1a)

| | | #drug | | | genbank protein |
|---|---|---|---|---|---|
| POS | drug class | classes | p-value | gene name | accession number |
| 3763210 | Other(*1); polyketide(*2); quinolone(*3); | 5 | 1.9784E−152 | parC | YP_001337063.1 |
| | Lactams; aminoglycoside | | | | |
| 1784305 | Other(*1); polyketide(*2); quinolone(*3); | 5 | 1.5316E−115 | KPN_01607 | YP_001335268.1 |
| | Lactams; aminoglycoside | | | | |
| 1784302 | Other(*1); polyketide(*2); quinolone(*3); | 5 | 8.1983E−115 | KPN_01607 | YP_001335268.1 |
| | Lactams; aminoglycoside | | | | |
| 2905411 | Other(*1); polyketide(*2); quinolone(*3); | 5 | 4.3727E−106 | gyrA | YP_001336287.1 |
| | Lactams; aminoglycoside | | | | |
| 2673906 | Other(*1); polyketide(*2); quinolone(*3); | 5 | 5.0133E−104 | KPN_02451 | YP_001336099.1 |
| | Lactams; aminoglycoside | | | | |
| 2773232 | Other(*1); polyketide(*2); quinolone(*3); | 5 | 5.5237E−104 | baeR | YP_001336179.1 |
| | Lactams; aminoglycoside | | | | |
| 140517 | Other(*1); polyketide(*2); quinolone(*3); | 5 | 1.3942E−103 | aceF | YP_001333809.1 |
| | Lactams; aminoglycoside | | | | |
| 809148 | Other(*1); polyketide(*2); quinolone(*3); | 5 | 1.3942E−103 | ybgH | YP_001334393.1 |
| | Lactams; aminoglycoside | | | | |
| 1364586 | Other(*1); polyketide(*2); quinolone(*3); | 5 | 1.3942E−103 | ynjE | YP_001334876.1 |
| | Lactams; aminoglycoside | | | | |
| 2150691 | Other(*1); polyketide(*2); quinolone(*3); | 5 | 1.3942E−103 | KPN_01951 | YP_001335612.1 |
| | Lactams; aminoglycoside | | | | |
| 2159024 | Other(*1); polyketide(*2); quinolone(*3); | 5 | 1.3942E−103 | KPN_01961 | YP_001335622.1 |
| | Lactams; aminoglycoside | | | | |
| 2317024 | Other(*1); polyketide(*2); quinolone(*3); | 5 | 1.3942E−103 | KPN_02114 | YP_001335772.1 |
| | Lactams; aminoglycoside | | | | |
| 2325877 | Other(*1); polyketide(*2); quinolone(*3); | 5 | 1.3942E−103 | mhpA | YP_001335780.1 |
| | Lactams; aminoglycoside | | | | |
| 2331649 | Other(*1); polyketide(*2); quinolone(*3); | 5 | 1.3942E−103 | KPN_02128 | YP_001335786.1 |
| | Lactams; aminoglycoside | | | | |
| 2347930 | Other(*1); polyketide(*2); quinolone(*3); | 5 | 1.3942E−103 | KPN_02144 | YP_001335802.1 |
| | Lactams; aminoglycoside | | | | |
| 2355785 | Other(*1); polyketide(*2); quinolone(*3); | 5 | 1.3942E−103 | KPN_02149 | YP_001335807.1 |
| | Lactams; aminoglycoside | | | | |
| 2365629 | Other(*1); polyketide(*2); quinolone(*3); | 5 | 1.3942E−103 | ydiJ | YP_001335816.1 |
| | Lactams; aminoglycoside | | | | |
| 2375692 | Other(*1); polyketide(*2); quinolone(*3); | 5 | 1.3942E−103 | btuE | YP_001335825.1 |
| | Lactams; aminoglycoside | | | | |
| 2402871 | Other(*1); polyketide(*2); quinolone(*3); | 5 | 1.3942E−103 | oppC | YP_001335853.1 |
| | Lactams; aminoglycoside | | | | |
| 2459360 | Other(*1); polyketide(*2); quinolone(*3); | 5 | 1.3942E−103 | pth | YP_001335898.1 |
| | Lactams; aminoglycoside | | | | |
| 2517274 | Other(*1); polyketide(*2); quinolone(*3); | 5 | 1.3942E−103 | KPN_02298 | YP_001335954.1 |
| | Lactams; aminoglycoside | | | | |
| 2521829 | Other(*1); polyketide(*2); quinolone(*3); | 5 | 1.3942E−103 | KPN_02302 | YP_001335958.1 |
| | Lactams; aminoglycoside | | | | |
| 2532012 | Other(*1); polyketide(*2); quinolone(*3); | 5 | 1.3942E−103 | dadA | YP_001335965.1 |
| | Lactams; aminoglycoside | | | | |
| 2547536 | Other(*1); polyketide(*2); quinolone(*3); | 5 | 1.3942E−103 | yoaA | YP_001335981.1 |
| | Lactams; aminoglycoside | | | | |
| 2629283 | Other(*1); polyketide(*2); quinolone(*3); | 5 | 1.3942E−103 | ftn | YP_001336058.1 |
| | Lactams; aminoglycoside | | | | |
| 2658497 | Other(*1); polyketide(*2); quinolone(*3); | 5 | 1.3942E−103 | cbl | YP_001336087.1 |
| | Lactams; aminoglycoside | | | | |
| 2703286 | Other(*1); polyketide(*2); quinolone(*3); | 5 | 1.3942E−103 | hisB | YP_001336126.1 |
| | Lactams; aminoglycoside | | | | |
| 2774521 | Other(*1); polyketide(*2); quinolone(*3); | 5 | 1.3942E−103 | yegQ | YP_001336180.1 |
| | Lactams; aminoglycoside | | | | |
| 2812941 | Other(*1); polyketide(*2); quinolone(*3); | 5 | 1.3942E−103 | yehY | YP_001336214.1 |
| | Lactams; aminoglycoside | | | | |
| 2831238 | Other(*1); polyketide(*2); quinolone(*3); | 5 | 1.3942E−103 | KPN_02580 | YP_001336228.1 |
| | Lactams; aminoglycoside | | | | |
| 2875745 | Other(*1); polyketide(*2); quinolone(*3); | 5 | 1.3942E−103 | yejH | YP_001336265.1 |
| | Lactams; aminoglycoside | | | | |
| 2878878 | Other(*1); polyketide(*2); quinolone(*3); | 5 | 1.3942E−103 | KPN_02621 | YP_001336269.1 |
| | Lactams; aminoglycoside | | | | |
| 2920245 | Other(*1); polyketide(*2); quinolone(*3); | 5 | 1.3942E−103 | yfaW | YP_001336299.1 |
| | Lactams; aminoglycoside | | | | |
| 2379716 | Other(*1); polyketide(*2); quinolone(*3); | 5 | 1.4844E−103 | KPN_02170 | YP_001335828.1 |
| | Lactams; aminoglycoside | | | | |
| 2218319 | Other(*1); polyketide(*2); quinolone(*3); | 5 | 1.5333E−103 | KPN_02025 | YP_001335683.1 |
| | Lactams; aminoglycoside | | | | |
| 2504346 | Other(*1); polyketide(*2); quinolone(*3); | 5 | 1.5333E−103 | livG | YP_001335944.1 |
| | Lactams; aminoglycoside | | | | |
| 2505230 | Other(*1); polyketide(*2); quinolone(*3); | 5 | 1.5333E−103 | livM | YP_001335945.1 |
| | Lactams; aminoglycoside | | | | |
| 2506816 | Other(*1); polyketide(*2); quinolone(*3); | 5 | 1.5333E−103 | livH | YP_001335946.1 |
| | Lactams; aminoglycoside | | | | |
| 2641631 | Other(*1); polyketide(*2); quinolone(*3); | 5 | 1.5333E−103 | fliY | YP_001336071.1 |
| | Lactams; aminoglycoside | | | | |
| 2646728 | Other(*1); polyketide(*2); quinolone(*3); | 5 | 1.5333E−103 | yedQ | YP_001336077.1 |
| | Lactams; aminoglycoside | | | | |
| 1704769 | Other(*1); polyketide(*2); quinolone(*3); | 5 | 1.6366E−103 | abgB | YP_001335194.1 |
| | Lactams; aminoglycoside | | | | |
| 2524562 | Other(*1); polyketide(*2); quinolone(*3); | 5 | 1.7489E−103 | treA | YP_001335959.1 |
| | Lactams; aminoglycoside | | | | |
| 2772839 | Other(*1); polyketide(*2); quinolone(*3); | 5 | 1.7911E−103 | baeS | YP_001336178.1 |
| | Lactams; aminoglycoside | | | | |
| 2627362 | Other(*1); polyketide(*2); quinolone(*3); | 5 | 1.8073E−103 | KPN_02399 | YP_001336055.1 |
| | Lactams; aminoglycoside | | | | |
| 2124017 | Other(*1); polyketide(*2); quinolone(*3); | 5 | 1.853E−103 | ydcR | YP_001335590.1 |
| | Lactams; aminoglycoside | | | | |
| 2174754 | Other(*1); polyketide(*2); quinolone(*3); | 5 | 1.853E−103 | anmK | YP_001335639.1 |
| | Lactams; aminoglycoside | | | | |
| 2275805 | Other(*1); polyketide(*2); quinolone(*3); | 5 | 1.853E−103 | ccmF | YP_001335734.1 |
| | Lactams; aminoglycoside | | | | |
| 2662814 | Other(*1); polyketide(*2); quinolone(*3); | 5 | 1.853E−103 | KPN_02440 | YP_001336090.1 |
| | Lactams; aminoglycoside | | | | |
| 2784148 | Other(*1); polyketide(*2); quinolone(*3); | 5 | 1.853E−103 | KPN_02540 | YP_001336188.1 |
| | Lactams; aminoglycoside | | | | |
| 1933723 | Other(*1); polyketide(*2); quinolone(*3); | 5 | 1.9245E−103 | KPN_01752 | YP_001335413.1 |
| | Lactams; aminoglycoside | | | | |
| 4595554 | Other(*1); polyketide(*2); quinolone(*3); | 5 | 2.0467E−103 | KPN_04195 | YP_001337841.1 |
| | Lactams; aminoglycoside | | | | |

(*1)benzene derived/sulfonamide
(*2)particularly tetracycline
(*3)particularly fluoroquinolone

TABLE 3b
Detailed results for the genes in Example 1 for Klebsiella oxytoca
(corresponding to Table 1b)

| | | #drug | | | genbank protein |
|---|---|---|---|---|---|
| POS | drug class | classes | p-value | gene name | accession number |
| 5645611 | other (benzene derived)/sulfonamide; | 4 | 6.03526E−61 | KOX_26125 | YP_005021173.1 |
| | polyketide(*2); quinolone(*3); Lactams | | | | |
| 2887469 | Lactams | 1 | 8.3881E−41 | KOX_13365 | YP_005018636.1 |
| 2887473 | Lactams | 1 | 8.3881E−41 | KOX_13365 | YP_005018636.1 |
| 3631990 | Lactams | 1 | 8.3881E−41 | KOX_16735 | YP_005019308.1 |
| 5544665 | Lactams | 1 | 8.3881E−41 | KOX_25695 | YP_005021089.1 |
| 5544668 | Lactams | 1 | 8.3881E−41 | KOX_25695 | YP_005021089.1 |
| 2652345 | Lactams | 1 | 8.74389E−41 | KOX_12270 | YP_005018419.1 |
| 3260573 | Lactams | 1 | 1.34809E−40 | KOX_15055 | YP_005018974.1 |

(*2)particularly tetracycline
(*3)particularly fluoroquinolone

TABLE 4a
Detailed results for the genes in Example 1 for Klebsiella pneumoniae
(corresponding to Table 2a)

| | | | | #drug |
|---|---|---|---|---|
| POS | drug | #drugs | drug class | classes |
| 3763210 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 1784305 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 1784302 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2905411 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2673906 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2773232 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 140517 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 809148 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 1364586 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2150691 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2159024 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2317024 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2325877 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2331649 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2347930 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2355785 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2365629 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2375692 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2402871 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2459360 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2517274 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2521829 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2532012 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2547536 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2629283 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2658497 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2703286 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2774521 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2812941 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2831238 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2875745 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2878878 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2920245 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2379716 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2218319 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2504346 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2505230 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2506816 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2641631 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2646728 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 1704769 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2524562 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2772839 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2627362 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2124017 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2174754 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2275805 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2662814 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 2784148 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 1933723 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |
| 4595554 | CF; T/S; TE; CFT; LVX; GM; IMP; CFZ; CRM; ETP; CP; | 21 | Other(*1); polyketide(*2); quinolone(*3); | 5 |
| | CAX; AZT; P/T; CPE; AM; A/S; CAZ; TO; MER; AUG | | Lactams; aminoglycoside | |

(*1)benzene derived/sulfonamide
(*2)particularly tetracycline
(*3)particularly fluoroquinolone

TABLE 4b
Detailed results for the genes in Example 1 for Klebsiella pneumoniae (corresponding
to Table 2a, continued)

| | | | | | #significant | #significant other |
|---|---|---|---|---|---|---|
| | best | #significant | #significant | #significant | polyketide | (benzene derived)/ |
| POS | drug | Lactams | fluoroquinolones | aminoglycosides | (tetracycline) | sulfonamide |
| 3763210 | CP | 15 | 2 | 2 | 1 | 1 |
| 1784305 | CFT | 15 | 2 | 2 | 1 | 1 |
| 1784302 | AZT | 15 | 2 | 2 | 1 | 1 |
| 2905411 | CP | 15 | 2 | 2 | 1 | 1 |
| 2673906 | IMP | 15 | 2 | 2 | 1 | 1 |
| 2773232 | IMP | 15 | 2 | 2 | 1 | 1 |
| 140517 | IMP | 15 | 2 | 2 | 1 | 1 |
| 809148 | IMP | 15 | 2 | 2 | 1 | 1 |
| 1364586 | IMP | 15 | 2 | 2 | 1 | 1 |
| 2150691 | IMP | 15 | 2 | 2 | 1 | 1 |
| 2159024 | IMP | 15 | 2 | 2 | 1 | 1 |
| 2317024 | IMP | 15 | 2 | 2 | 1 | 1 |
| 2325877 | IMP | 15 | 2 | 2 | 1 | 1 |
| 2331649 | IMP | 15 | 2 | 2 | 1 | 1 |
| 2347930 | IMP | 15 | 2 | 2 | 1 | 1 |
| 2355785 | IMP | 15 | 2 | 2 | 1 | 1 |
| 2365629 | IMP | 15 | 2 | 2 | 1 | 1 |
| 2375692 | IMP | 15 | 2 | 2 | 1 | 1 |
| 2402871 | IMP | 15 | 2 | 2 | 1 | 1 |
| 2459360 | IMP | 15 | 2 | 2 | 1 | 1 |
| 2517274 | IMP | 15 | 2 | 2 | 1 | 1 |
| 2521829 | IMP | 15 | 2 | 2 | 1 | 1 |
| 2532012 | IMP | 15 | 2 | 2 | 1 | 1 |
| 2547536 | IMP | 15 | 2 | 2 | 1 | 1 |
| 2629283 | IMP | 15 | 2 | 2 | 1 | 1 |
| 2658497 | IMP | 15 | 2 | 2 | 1 | 1 |
| 2703286 | IMP | 15 | 2 | 2 | 1 | 1 |
| 2774521 | IMP | 15 | 2 | 2 | 1 | 1 |
| 2812941 | IMP | 15 | 2 | 2 | 1 | 1 |
| 2831238 | IMP | 15 | 2 | 2 | 1 | 1 |
| 2875745 | IMP | 15 | 2 | 2 | 1 | 1 |
| 2878878 | IMP | 15 | 2 | 2 | 1 | 1 |
| 2920245 | IMP | 15 | 2 | 2 | 1 | 1 |
| 2379716 | IMP | 15 | 2 | 2 | 1 | 1 |
| 2218319 | IMP | 15 | 2 | 2 | 1 | 1 |
| 2504346 | IMP | 15 | 2 | 2 | 1 | 1 |
| 2505230 | IMP | 15 | 2 | 2 | 1 | 1 |
| 2506816 | IMP | 15 | 2 | 2 | 1 | 1 |
| 2641631 | IMP | 15 | 2 | 2 | 1 | 1 |
| 2646728 | IMP | 15 | 2 | 2 | 1 | 1 |
| 1704769 | IMP | 15 | 2 | 2 | 1 | 1 |
| 2524562 | IMP | 15 | 2 | 2 | 1 | 1 |
| 2772839 | IMP | 15 | 2 | 2 | 1 | 1 |
| 2627362 | IMP | 15 | 2 | 2 | 1 | 1 |
| 2124017 | IMP | 15 | 2 | 2 | 1 | 1 |
| 2174754 | IMP | 15 | 2 | 2 | 1 | 1 |
| 2275805 | IMP | 15 | 2 | 2 | 1 | 1 |
| 2662814 | IMP | 15 | 2 | 2 | 1 | 1 |
| 2784148 | IMP | 15 | 2 | 2 | 1 | 1 |
| 1933723 | IMP | 15 | 2 | 2 | 1 | 1 |
| 4595554 | CP | 15 | 2 | 2 | 1 | 1 |

TABLE 4c
Detailed results for the genes in Example 1 for Klebsiella pneumoniae
(corresponding to Table 2a, continued)

| | | | genbank protein |
|---|---|---|---|
| POS | p-value | gene name | accession number |
| 3763210 | 1.9784E−152 | parC | YP_001337063.1 |
| 1784305 | 1.5316E−115 | KPN_01607 | YP_001335268.1 |
| 1784302 | 8.1983E−115 | KPN_01607 | YP_001335268.1 |
| 2905411 | 4.3727E−106 | gyrA | YP_001336287.1 |
| 2673906 | 5.0133E−104 | KPN_02451 | YP_001336099.1 |
| 2773232 | 5.5237E−104 | baeR | YP_001336179.1 |
| 140517 | 1.3942E−103 | aceF | YP_001333809.1 |
| 809148 | 1.3942E−103 | ybgH | YP_001334393.1 |
| 1364586 | 1.3942E−103 | ynjE | YP_001334876.1 |
| 2150691 | 1.3942E−103 | KPN_01951 | YP_001335612.1 |
| 2159024 | 1.3942E−103 | KPN_01961 | YP_001335622.1 |
| 2317024 | 1.3942E−103 | KPN_02114 | YP_001335772.1 |
| 2325877 | 1.3942E−103 | mhpA | YP_001335780.1 |
| 2331649 | 1.3942E−103 | KPN_02128 | YP_001335786.1 |
| 2347930 | 1.3942E−103 | KPN_02144 | YP_001335802.1 |
| 2355785 | 1.3942E−103 | KPN_02149 | YP_001335807.1 |
| 2365629 | 1.3942E−103 | ydiJ | YP_001335816.1 |
| 2375692 | 1.3942E−103 | btuE | YP_001335825.1 |
| 2402871 | 1.3942E−103 | oppC | YP_001335853.1 |
| 2459360 | 1.3942E−103 | pth | YP_001335898.1 |
| 2517274 | 1.3942E−103 | KPN_02298 | YP_001335954.1 |
| 2521829 | 1.3942E−103 | KPN_02302 | YP_001335958.1 |
| 2532012 | 1.3942E−103 | dadA | YP_001335965.1 |
| 2547536 | 1.3942E−103 | yoaA | YP_001335981.1 |
| 2629283 | 1.3942E−103 | ftn | YP_001336058.1 |
| 2658497 | 1.3942E−103 | cbl | YP_001336087.1 |
| 2703286 | 1.3942E−103 | hisB | YP_001336126.1 |
| 2774521 | 1.3942E−103 | yegQ | YP_001336180.1 |
| 2812941 | 1.3942E−103 | yehY | YP_001336214.1 |
| 2831238 | 1.3942E−103 | KPN_02580 | YP_001336228.1 |
| 2875745 | 1.3942E−103 | yejH | YP_001336265.1 |
| 2878878 | 1.3942E−103 | KPN_02621 | YP_001336269.1 |
| 2920245 | 1.3942E−103 | yfaW | YP_001336299.1 |
| 2379716 | 1.4844E−103 | KPN_02170 | YP_001335828.1 |
| 2218319 | 1.5333E−103 | KPN_02025 | YP_001335683.1 |
| 2504346 | 1.5333E−103 | livG | YP_001335944.1 |
| 2505230 | 1.5333E−103 | livM | YP_001335945.1 |
| 2506816 | 1.5333E−103 | livH | YP_001335946.1 |
| 2641631 | 1.5333E−103 | fliY | YP_001336071.1 |
| 2646728 | 1.5333E−103 | yedQ | YP_001336077.1 |
| 1704769 | 1.6366E−103 | abgB | YP_001335194.1 |
| 2524562 | 1.7489E−103 | treA | YP_001335959.1 |
| 2772839 | 1.7911E−103 | baeS | YP_001336178.1 |
| 2627362 | 1.8073E−103 | KPN_02399 | YP_001336055.1 |
| 2124017 | 1.853E−103 | ydcR | YP_001335590.1 |
| 2174754 | 1.853E−103 | anmK | YP_001335639.1 |
| 2275805 | 1.853E−103 | ccmF | YP_001335734.1 |
| 2662814 | 1.853E−103 | KPN_02440 | YP_001336090.1 |
| 2784148 | 1.853E−103 | KPN_02540 | YP_001336188.1 |
| 1933723 | 1.9245E−103 | KPN_01752 | YP_001335413.1 |
| 4595554 | 2.0467E−103 | KPN_04195 | YP_001337841.1 |

TABLE 4d
Detailed results for the genes in Example 1 for Klebsiella oxytoca
(corresponding to Table 2b)

| | | | | #drug |
|---|---|---|---|---|
| POS | drug | #drugs | drug class | classes |
| 5645611 | CF; T/S; TE; CFT; CFZ; CRM; | 16 | other (benzene derived)/ | 4 |
| | CP; CAX; AZT; P/T; CPE; AM; | | sulfonamide; polyketide(*2); | |
| | A/S; CAZ; LVX; AUG | | quinolone(*3); Lactams | |
| 2887469 | CF; CFZ; CRM; AZT; AM; A/S | 6 | Lactams | 1 |
| 2887473 | CF; CFZ; CRM; AZT; AM; A/S | 6 | Lactams | 1 |
| 3631990 | CF; CFZ; CRM; AZT; AM; A/S | 6 | Lactams | 1 |
| 5544665 | CF; CFZ; CRM; AZT; AM; A/S | 6 | Lactams | 1 |
| 5544668 | CF; CFZ; CRM; AZT; AM; A/S | 6 | Lactams | 1 |
| 2652345 | CF; CFZ; CRM; AZT; AM; A/S | 6 | Lactams | 1 |
| 3260573 | CF; CFZ; CRM; AZT; AM; A/S | 6 | Lactams | 1 |

TABLE 4e
Detailed results for the genes in Example 1 for Klebsiella oxytoca (corresponding to
Table 2b, continued)

| | | | | | #significant | #significant other |
|---|---|---|---|---|---|---|
| | best | #significant | #significant | #significant | polyketide | (benzene derived)/ |
| POS | drug | Lactams | fluoroquinolones | aminoglycosides | (tetracycline) | sulfonamide |
| 5645611 | LVX | 12 | 2 | 0 | 1 | 1 |
| 2887469 | CFZ | 6 | 0 | 0 | 0 | 0 |
| 2887473 | CFZ | 6 | 0 | 0 | 0 | 0 |
| 3631990 | CFZ | 6 | 0 | 0 | 0 | 0 |
| 5544665 | CFZ | 6 | 0 | 0 | 0 | 0 |
| 5544668 | CFZ | 6 | 0 | 0 | 0 | 0 |
| 2652345 | CFZ | 6 | 0 | 0 | 0 | 0 |
| 3260573 | CFZ | 6 | 0 | 0 | 0 | 0 |

TABLE 4f
Detailed results for the genes in Example 1 for Klebsiella oxytoca
(corresponding to Table 2b, continued)

| | | | genbank protein |
|---|---|---|---|
| POS | p-value | gene name | accession number |
| 5645611 | 6.03526E−61 | KOX_26125 | YP_005021173.1 |
| 2887469 | 8.3881E−41 | KOX_13365 | YP_005018636.1 |
| 2887473 | 8.3881E−41 | KOX_13365 | YP_005018636.1 |
| 3631990 | 8.3881E−41 | KOX_16735 | YP_005019308.1 |
| 5544665 | 8.3881E−41 | KOX_25695 | YP_005021089.1 |
| 5544668 | 8.3881E−41 | KOX_25695 | YP_005021089.1 |
| 2652345 | 8.74389E−41 | KOX_12270 | YP_005018419.1 |
| 3260573 | 1.34809E−40 | KOX_15055 | YP_005018974.1 |

Also the antibiotic/drug classes, the number of significant antibiotics correlated to the mutations (over all antibiotics or over certain classes), as well as the correlated antibiotics are denoted in the Tables.

The p-value was calculated using the fisher exact test based on contingency table with 4 fields: #samples Resistant/wild type; #samples Resistant/mutant; #samples not Resistant/wild type; #samples not Resistant/mutant

The test is based on the distribution of the samples in the 4 fields. Even distribution indicates no significance, while clustering into two fields indicates significance.

The following results were obtained for Klebsiella pneumoniae:
A total of 38,225 different correlations between genetic sites and anti-microbial agents were detected (p-value <10⁻¹¹).
The biggest part of these were point mutations (i.e. single base exchanges)
The highest significance that was reached was 10⁻¹⁵⁹ for a mutation in YP_001337063.1, particularly in position 3763210 with regard to reference genome NC_009648 as annotated at the NCBI, particularly being a codon change aGc/aTc
Besides these, insertions or deletions of up to four bases were discovered
Further, potential genetic tests for five different drug classes relating to resistances were discovered
   β-lactams (includes Penicillins, Cephalosporins, Carbapenems, Monobactams)
   Quinolones, particularly Fluoroquinolones
   Aminoglycosides
   Polyketides, particularly Tetracyclines
   Folate synthesis inhibitors
Potential genetic tests for all tested drugs/drug combinations were discovered:
   Amoxicillin/Clavulanate, Ampicillin, Ampicillin/Sulbactam, Aztreonam, Cefazolin, Cefepime, Ceftazidime, Cefuroxime, Cephalothin, Imipenem, Piperacillin/Tazobactam, Ciprofloxacin, Levofloxacin, Gentamycin, Tobramycin, Tetracycline, Trimethoprim/Sulfamethoxazol
Mutations were observed in 4,053 different genes

The following results were obtained for Klebsiella oxytoca:
A total of 74,088 different correlations between genetic sites and anti-microbial agents were detected (p-value <10⁻¹¹).
The biggest part of these were point mutations (i.e. single base exchanges)
The highest significance that was reached was 10⁻⁶⁷ for a mutation in YP_005021173.1, particularly in position 5645611 with regard to reference genome NC_016612 as annotated at the NCBI, particularly being a codon change aCt/aTt
Besides these, insertions or deletions of up to four bases were discovered
Further, potential genetic tests for four different drug classes relating to resistances were discovered
   β-lactams (includes Penicillins, Cephalosporins, Carbapenems, Monobactams)
   Quinolones, particularly Fluoroquinolones
   Polyketides, particularly Tetracyclines
   Folate synthesis inhibitors
Potential genetic tests for the tested drugs/drug combinations were discovered:
   Amoxicillin/Clavulanate, Ampicillin, Ampicillin/Sulbactam, Aztreonam, Cefazolin, Cefepime, Ceftazidime, Cefuroxime, Cephalothin, Imipenem, Piperacillin/Tazobactam, Ciprofloxacin, Levofloxacin, Gentamycin, Tobramycin, Tetracycline, Trimethoprim/Sulfamethoxazol
Mutations were observed in 4,599 different genes

### Example 2

In addition to the 1,176 K. pneumoniae isolates, we generated genetic profiles for 1,162 pathogenic E. coli isolates from the microbiology strain collection at Siemens Healthcare Diagnostics (West Sacramento, Calif.) for susceptibility testing and whole genome sequencing by using whole genome next-generation sequencing with the same method as described in Example 1, unless noted otherwise. For the same isolates we performed culture based resistance tests for 21 different drugs as current gold standard. Next we calculated genome wide association between genotypes and resistance profiles. Following systematic analysis of genetic and culture based data we compared both genera to identify common resistance mechanisms.

### Data Analysis

Data analysis was carried out for E. coli as in Example 1, except for the following differences.

For further analyses positions with non-synonymous alterations and p-value <10⁻⁹ were considered. Based on the contingency table, the accuracy (ACC), sensitivity (SENS), specificity (SPEC), and the positive/negative predictive values (PPV/NPV) were calculated, which are shown in FIG. 2. FIG. 2 shows an exemplary contingency table for the computation of the Fisher's exact test and the measures accuracy, sensitivity, specificity, positive predictive value (PPV), and negative predictive value (NPV). Numbers are given for amino acid exchange S83L (GyrA) and Ciprofloxacin in E. coli.

For E. coli, gene ranges were extracted from the reference assembly NC_010473.gff.

We report 25,646 non-synonymous sites in the E. coli genome that are significantly correlated to drug resistance (p<10⁻⁹). Highest significance was reached for the drugs Ciprofloxacin and Levofloxacin with respect to the amino acid (AA) exchange S83L in the drug target DNA gyrase A (p=10⁻²³⁵, accuracy, specificity and sensitivity: 98%, 99%, and 94%). The second most significant association was observed for S80I of DNA topoisomerase IV subunit A (ParC), another target for quinolone antibiotics (p=10⁻¹⁹⁶).

### Example 3

For comparing mutations in E. coli (Example 2) and K. pneumoniae (from Example 1) in genes with high similarity, we first performed a pairwise protein BLAST with all amino acid sequences from E. coli and K. pneumoniae. Afterwards, we filtered the matches for having at least 80% positives (identical AAs (amino acids) and AAs that have similar properties) and the smaller sequence in the comparison having an overlap with the alignment of at least 90% of its length. In an additional filtering step, we only kept mappings where the official gene name for both genes were the same. To find now overlapping mutations associated with drug resistance in E. coli and K. pneumoniae, we extracted the gene names and the amino acid exchanges for both organisms and intersected the two lists. The resulting list was additionally matched with the gene names from the BLAST list to only keep functionally similar hits.

The resistance classification of the E. coli and K. pneumoniae isolates was performed using non-synonymous SNPs as categorical features. For each sample we have calculated the number of features with missing values. Isolates with more than 25% of missing data are removed resulting in 1,151 samples for E. coli and 1,176 for K. pneumonia, respectively.

To improve prediction of resistance combinations of mutations can be used. Thus, decision trees were built to classify samples as resistant or not resistant for each drug separately using the R package rpart for model training and prediction. The samples were classified as resistant or not resistant with respect to each of the 21 drugs based on the breakpoint table of the European Committee on Antimicrobial Susceptibility Testing (EUCAST, Version 4.0, 2014, Enterobacteriaceae). Three of the 21 drugs (Cefalotin, Cefazolin, and Tetracycline) have no breakpoints specified in the EUCAST table and were not considered for resistance prediction. Additionally, drugs with less than 10 resistant isolates in the data set were omitted (Meropenem, Imipenem for E. coli, none for K. pneumoniae). To assess how well the classifiers can generalize to an independent data set, we performed 5-fold cross-validation (repeated 10 times), and computed the average performance values and their standard deviation. Afterwards, the final models were built on the complete data set. To account for class imbalance, the decision trees are constructed using a loss matrix computed with respect to class proportions.

For E. coli, the results are as above in Example 2. For K. pneumoniae we report the sites as above correlated to drug resistance. These showed a high concordance to the E. coli mutations. In 55 cases even the identical AA exchange was observed. One example is the most significant K. pneumoniae AA exchange S80I in ParC (p=10-160, accuracy, specificity, and sensitivity: 97%, 100%, and 83%). Besides exchanges of single AAs, we discovered gene dosage effects of several genes, e.g. an increased coverage of β-lactamase in K. pneumoniae for resistant isolates.

TABLE 13
Antibiotic Drugs

| | Accession | | |
|---|---|---|---|
| Drug | Number | Abbreviation | Class |
| Ampicillin/Sulbactam | DB00415 | A/S | Lactams |
| Ampicillin | DB00415 | AM | Lactams |
| Amoxicillin | DB01060; | AUG | Lactams |
| Clavulanate | DB00766 | | |
| Aztreonam | DB00355 | AZT | Lactams |
| Ceftriaxone | DB01212 | CAX | Lactams |
| Ceftazidime | DB00438 | CAZ | Lactams |
| Cefalotin | DB00456 | CF | Lactams |
| Cefotaxime | DB00493 | CFT | Lactams |
| Cefazolin | DB01327 | CFZ | Lactams |
| Ciprofloxacin | DB00537 | CP | fluoroquinolone |
| Cefepime | DB01413 | CPE | Lactams |
| Cefuroxime | DB01112 | CRM | Lactams |
| Ertapenem | DB00303 | ETP | Lactams |
| Gentamicin | DB00798 | GM | aminoglycoside |
| Imipenem | DB01598 | IMP | Lactams |
| Levofloxacin | DB01137 | LVX | fluoroquinolone |
| Meropenem | DB00760 | MER | Lactams |
| Piperacillin | DB00319; | P/T | Lactams |
| Tazobactam | DB01606 | | |
| Trimethoprim | DB00440; | T/S | other (benzene |
| Sulfamethoxazole | DB01015 | | derived)/sulfonamide |
| Tetracycline | DB00759 | TE | polyketide |
| | | | (tetracycline) |
| Tobramycin | DB00684 | TO | aminoglycoside |

Further results for Klebsiella pneumoniae from Example 1 and E. coli from Example 2 as well as from Example 3 that compare the results for these species are given in the following.

To improve the understanding of genetic resistance mechanisms of pathogenic bacteria we performed culture-based AST for 1,162 E. coli and 1,176 K. pneumoniae isolates and 21 antimicrobial drugs belonging to 5 different drug classes: β-lactams, fluoroquinolones, aminoglycosides, tetracyclines, and folate synthesis inhibitor. The complete list of drugs is as above and is also given below in Table 13. The complete list of E. coli and K. pneumoniae, as well as also K. oxytoca, isolates is available but not listed herein. For the same isolates we performed whole genome sequencing and genome wide correlation of genetic variants to culture based resistance tests and compared the results of E. coli and K. pneumoniae.

Most significant sites in the E. coli and K. pneumoniae genome

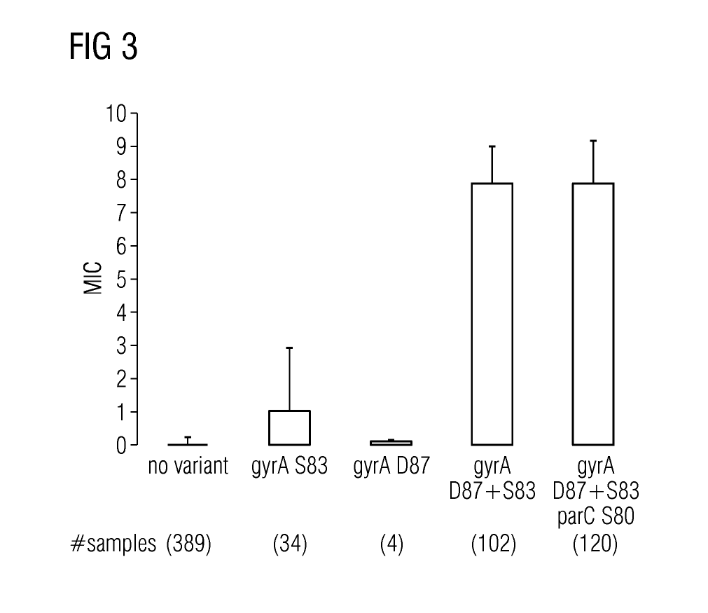

In order to calculate genome-wide significance scores, we mapped all 1,162 E. coli genomes to the reference strain DH10B. For each genomic position we determined the base for each sample and discovered 973,226 sites that passed the quality filtering and in which at least one sample had a non-reference base. The respective sites were correlated to the AST data for the 21 drugs using Fisher's exact test. Our analysis revealed 25,646 sites where a genetic mutation significantly correlated with at least one drug (p-value<10⁻⁹) and led to a change in the AA sequence, including point mutation and small insertions and deletions. The highest significance was reached for AA exchange S83L in GyrA and the drug Ciprofloxacin (p=10⁻²³⁵). Remarkably, GyrA is one of the targets of Ciprofloxacin. For this position, three AA exchanges, S83L, S83W, S83A, are annotated in UniProt as conferring resistance to quinolones. Here, only 5 false positive (0.4%) and 18 false negative samples (1.6%) were discovered while 1,139 samples were identified correctly, corresponding to accuracy, specificity, and sensitivity of 98.0%, 99.4% and 93.8%, respectively, see FIG. 2. Similarly, the second most significant site in GyrA, D87N/D87Y, revealed just 12 false positives and 10 false negatives. The respective p-value was 10⁻²⁰⁶ and the accuracy 98.1%. Again, for this site the D87N exchange is annotated as conferring quinolone resistance in UniProt. For the third and fourth most significant sites, located in the second Ciprofloxacin target, ParC, (S80I, E84G), resistance related variants have also been described. In FIG. 3, we present the means and standard deviations of MICs for Ciprofloxacin for samples having no variant in GyrA (S83/D87) and ParC (S80), samples having only one mutation either in GyrA S83 or D87 and not ParC, samples having both mutations in GyrA and not ParC, and samples having all three mutations. The mean MIC values increase from below 1.0 for no or single mutants to above 7.8 for double or triple mutants, highlighting a cumulative effect of single mutations to reach a higher level of resistance.

Besides the mutations in type II topoisomerase drug targets (GyrA/ParC), mutations in genes ygiF (A110T, p=10⁻⁶⁷, acc=86%, spec=89.5%, sens=69.9%) and ygjM (A68V, p=10⁻⁶³, acc=89.9%, spec=94.4%, sens=67.1%) have also a high significance. Compared to the above-described AA exchanges, these two sites demonstrate a substantially decreased sensitivity and positive predictive value (PPV). While the PPV for the four AA exchanges in GyrA and ParC was between 94.8% and 98.2%, the PPV of these two exchanges decreases to 59.0% and 70.8%. This means that the likelihood to be resistant given the exchanged AA is almost as high as the likelihood to be susceptible given the exchanged AA, limiting the probability that the respective AA exchanges are causative.

To discover other AA exchanges that are potentially causative for drug resistance, we filtered the list of all 25,646 sites (at least 150 resistant E. coli isolates carry the AA exchange, NPV>50%, PPV>75%). This filtering revealed 127 candidate sites, which are shown in Table 14.

TABLE 14
E. coli filtered sites

| | | best | | amino acid | gene | genbank protein |
|---|---|---|---|---|---|---|
| POS | drug | drug | p-value | change | name | accession number |
| 186619 | CF; A/S; AM; AUG | CF MIC | 6.13E−015 | T62R; T62K | tilS | YP_001729144.1 |
| 211929 | CF; CFZ; AUG; AM; A/S | AUG | 2.46E−020 | M164T | yafT | YP_001729167.1 |
| | | MIC | | | | |
| 274889 | CF; T/S; A/S; AM; CFZ; | CF MIC | 1.97E−012 | S274T | yagR | YP_001729228.1 |
| | AUG | | | | | |
| 469160 | CF; CFZ; AUG; AM; A/S | CF MIC | 2.03E−025 | N208D | ybbB | YP_001729406.1 |
| 782577 | CF; AUG | AUG | 2.94E−013 | N394D | rhsC | YP_001729688.1 |
| | | MIC | | | | |
| 782873 | CF; TE; CFT; CFZ; CRM; | AUG | 1.10E−024 | W492C | rhsC | YP_001729688.1 |
| | AZT; P/T; AM; A/S; | MIC | | | | |
| | CAZ; AUG | | | | | |
| 783207 | CF; AM; AUG | CF MIC | 1.07E−013 | T604A | rhsC | YP_001729688.1 |
| 783547 | CF; CFZ; AUG; AM; A/S | AUG | 7.79E−020 | R717Q | rhsC | YP_001729688.1 |
| | | MIC | | | | |
| 790070 | CF; CP; CFZ; LVX; AM; | CF MIC | 7.68E−021 | P55L | ybfD | YP_001729693.1 |
| | A/S; AUG | | | | | |
| 1051275 | CF; AUG | AUG | 2.57E−013 | T86I | ycbQ | YP_001729916.1 |
| | | MIC | | | | |
| 1054050 | CF; AM; AUG | AUG | 4.82E−019 | I562V; I562L | ycbS | YP_001729918.1 |
| | | MIC | | | | |
| 1054908 | CF; CFZ; AUG; AM; A/S | AUG | 2.21E−019 | E848Q; E848* | ycbS | YP_001729918.1 |
| | | MIC | | | | |
| 1057678 | CF; AUG | AUG | 7.34E−016 | V194A | ycbF | YP_001729922.1 |
| | | MIC | | | | |
| 1072811 | CF; CFZ; AUG; AM; A/S | CF MIC | 5.63E−025 | Y132H; Y132D | ompA | YP_001729935.1 |
| 1117663 | CF; AUG | CF MIC | 4.51E−012 | F159L | yccE | YP_001729980.1 |
| 1117727 | CF; AUG | CF MIC | 1.85E−011 | S181T; S181A | yccE | YP_001729980.1 |
| 1117967 | CF; A/S; AUG | AUG | 4.54E−012 | N261D | yccE | YP_001729980.1 |
| | | MIC | | | | |
| 1278853 | CF; CP; CFZ; LVX; AM; | CF MIC | 2.01E−024 | G171S | dadX | YP_001730138.1 |
| | A/S; AUG | | | | | |
| 1379108 | CF; AUG | CF MIC | 3.49E−012 | Q119; Q119H | cynX | YP_001730237.1 |
| 1387656 | CF; CP; TO; AUG | CF MIC | 6.72E−014 | V127L | mhpA | YP_001730242.1 |
| 1451283 | CF; AM; AUG | AUG | 2.45E−014 | G374E | puuC | YP_001730299.1 |
| | | MIC | | | | |
| 1453846 | TO | TO MIC | 6.03E−011 | L293F | puuE | YP_001730301.1 |
| 1453961 | CF; T/S; AM; A/S | CF MIC | 1.24E−012 | K331T | puuE | YP_001730301.1 |
| 1489600 | CF; AM; AUG | CF MIC | 4.50E−015 | V359A | abgB | YP_001730336.1 |
| 1489661 | CF; A/S; AM; AUG | AUG | 3.42E−019 | S339P | abgB | YP_001730336.1 |
| | | MIC | | | | |
| 1489740 | CF; A/S; AM; AUG | AUG | 3.27E−020 | Q312; Q312H | abgB | YP_001730336.1 |
| | | MIC | | | | |
| 1489958 | CF; AUG | CF MIC | 1.29E−012 | N240D | abgB | YP_001730336.1 |
| 1565547 | CF; AUG | AUG | 1.55E−016 | D595E | ydbD | YP_001730403.1 |
| | | MIC | | | | |
| 1565575 | CF; AUG | AUG | 8.19E−016 | I605V | ydbD | YP_001730403.1 |
| | | MIC | | | | |
| 1655695 | CF; A/S; AM; AUG | CF MIC | 4.61E−019 | P22L | yddV | YP_001730481.1 |
| 1691136 | CF; AM; AUG | AUG | 2.97E−018 | L157F | lsrC | YP_001730502.1 |
| | | MIC | | | | |
| 1693806 | CF; CFZ; AUG; AM; A/S | AUG | 1.13E−021 | K20Q | lsrF | YP_001730505.1 |
| | | MIC | | | | |
| 1703538 | CF; A/S; AM; AUG | AUG | 2.11E−019 | N43D | yneK | YP_001730514.1 |
| | | MIC | | | | |
| 1703689 | CF; A/S; AM; AUG | AUG | 1.63E−014 | A93V; A93D | yneK | YP_001730514.1 |
| | | MIC | | | | |
| 1901345 | CF; AM; AUG | AUG | 1.27E−016 | S132N | ydjO | YP_001730707.1 |
| | | MIC | | | | |
| 1901378 | CF; AM; AUG | CF MIC | 8.85E−017 | V121E | ydjO | YP_001730707.1 |
| 1901381 | CF; AM; AUG | CF MIC | 8.85E−017 | S120C | ydjO | YP_001730707.1 |
| 1901388 | CF; AM; AUG | CF MIC | 8.85E−017 | V118F | YdjO | YP_001730707.1 |
| 1901400 | CF; AM; AUG | CF MIC | 8.85E−017 | I114V | YdjO | YP_001730707.1 |
| 1901406 | CF; AM; AUG | CF MIC | 1.74E−016 | D112N | YdjO | YP_001730707.1 |
| 1901409 | CF; AM; AUG | CF MIC | 1.69E−016 | K111E | YdjO | YP_001730707.1 |
| 1971385 | CF; A/S; TE; AM; AUG | AUG | 1.61E−019 | N293; N293K | yeaU | YP_001730776.1 |
| | | MIC | | | | |
| 2235082 | CF; CFZ; AUG; AM; A/S | CF MIC | 6.46E−018 | −929 | yegE | YP_001731017.1 |
| 2401211 | CF; AUG | CF MIC | 1.18E−014 | −183T?; GFT181G | ompC | YP_001731155.1 |
| 2428172 | CF; T/S; TE; CFT; LVX; | CP MIC | 2.58E−206 | D87N; D87Y | gyrA | YP_001731169.1 |
| | GM; CFZ; CRM; ETP; CP; | | | | | |
| | CAX; AZT; P/T; CPE; | | | | | |
| | AM; A/S; CAZ; TO; AUG | | | | | |
| 2428183 | CF; T/S; TE; CFT; LVX; | CP MIC | 2.02E−235 | S83L | gyrA | YP_001731169.1 |
| | GM; CFZ; CRM; ETP; CP; | | | | | |
| | CAX; AZT; P/T; CPE; | | | | | |
| | AM; A/S; CAZ; TO; AUG | | | | | |
| 2432327 | CF; AUG | AUG | 1.23E−017 | S285T | yfaL | YP_001731171.1 |
| | | MIC | | | | |
| 2450226 | CF; CP; CFZ; LVX; AM; | CF MIC | 7.73E−029 | V71I | yfaW | YP_001731185.1 |
| | A/S; AUG | | | | | |
| 2473640 | CF; AM; AUG | AUG | 4.41E−017 | H381D | elaD | YP_001731207.1 |
| | | MIC | | | | |
| 2473652 | CF; AUG | AUG | 3.39E−014 | N385H; N385D | elaD | YP_001731207.1 |
| | | MIC | | | | |
| 2473679 | T/S; A/S; AM; AUG | A/S | 2.37E−013 | K394Q | elaD | YP_001731207.1 |
| | | MIC | | | | |
| 2539067 | CF | CF MIC | 1.87E−010 | M257T; M257R; | yfcO | YP_001731268.1 |
| | | | | M257K | | |
| 2553487 | CF; CFZ; AM; AUG | CF MIC | 7.07E−014 | S230N | yfdF | YP_001731281.1 |
| 2553661 | CF; AM; AUG | AUG | 8.68E−017 | Y288F | yfdF | YP_001731281.1 |
| | | MIC | | | | |
| 2553666 | CF; A/S; AM; AUG | AUG | 4.57E−017 | C290R | yfdF | YP_001731281.1 |
| | | MIC | | | | |
| 2553687 | CF; A/S; AM; AUG | AUG | 3.56E−017 | V297I | yfdF | YP_001731281.1 |
| | | MIC | | | | |
| 2553736 | CF; AM; AUG | AUG | 1.05E−016 | I313K | yfdF | YP_001731281.1 |
| | | MIC | | | | |
| 2553763 | CF; A/S; AM; AUG | AUG | 1.32E−017 | G322D | yfdF | YP_001731281.1 |
| | | MIC | | | | |
| 2553768 | CF; AM; AUG | AUG | 9.88E−017 | I324V | yfdF | YP_001731281.1 |
| | | MIC | | | | |
| 2553774 | CF; A/S; AM; AUG | AUG | 4.53E−017 | E326K | yfdF | YP_001731281.1 |
| | | MIC | | | | |
| 2553798 | CF; AM; AUG | AUG | 2.37E−014 | E334K | yfdF | YP_001731281.1 |
| | | MIC | | | | |
| 2584055 | CF; TE; CFZ; CP; P/T; | CF MIC | 7.05E−034 | AA44A | yfdX | YP_001731310.1 |
| | LVX; AM; A/S; CAZ; AUG | | | | | |
| 2693187 | AUG | AUG | 5.38E−013 | Q528R | hyfB | YP_001731412.1 |
| | | MIC | | | | |
| 2698423 | CF; AM; AUG | AUG | 1.24E−016 | Q50H | hyfG | YP_001731417.1 |
| | | MIC | | | | |
| 2700563 | CF; T/S; TE; CFZ; CP; | AUG | 1.38E−021 | R24L; R24Q | hyfI | YP_001731419.1 |
| | AM; A/S; AUG | MIC | | | | |
| 2956235 | CF; CFZ; AUG; AM; A/S | CF MIC | 2.12E−021 | V191I | ygbN | YP_001731635.1 |
| 3086789 | CF; AM; AUG | AUG | 1.92E−015 | A3S | ygeK | YP_001731742.1 |
| | | MIC | | | | |
| 3087957 | CF; AUG | CF MIC | 4.28E−013 | S108L | ygeO | YP_001731745.1 |
| 3261502 | CF; T/S; TE; CFT; LVX; | CP MIC | 1.81E−196 | S80I | parC | YP_001731882.1 |
| | GM; CFZ; CRM; ETP; CP; | | | | | |
| | CAX; AZT; P/T; CPE; | | | | | |
| | AM; A/S; CAZ; TO; AUG | | | | | |
| 3277903 | CF; CFZ; AUG; AM; A/S | CF MIC | 9.67E−024 | R91H | ygiD | YP_001731902.1 |
| 3348819 | CF; T/S; TE; CFZ; CP; | AUG | 1.93E−023 | L48F | yhaI | YP_001731966.1 |
| | AM; A/S; AUG | MIC | | | | |
| 3348826 | CF; T/S; TE; CFZ; CP; | AUG | 2.06E−023 | Y50F | yhaI | YP_001731966.1 |
| | LVX; AM; A/S; AUG | MIC | | | | |
| 3348834 | CF; TE; CFZ; CP; AM; | AUG | 9.73E−023 | M53L | yhaI | YP_001731966.1 |
| | A/S; AUG | MIC | | | | |
| 3348836 | CF; TE; CFZ; CP; AM; | AUG | 9.73E−023 | M53I | yhaI | YP_001731966.1 |
| | A/S; AUG | MIC | | | | |
| 3348837 | CF; TE; CFZ; CP; AM; | AUG | 9.73E−023 | L54I | yhaI | YP_001731966.1 |
| | A/S; AUG | MIC | | | | |
| 3348846 | CF; TE; CFZ; CP; AM; | AUG | 1.04E−022 | L57V | yhaI | YP_001731966.1 |
| | A/S; AUG | MIC | | | | |
| 3348847 | CF; TE; CFZ; CP; AM; | AUG | 1.04E−022 | L57P | yhaI | YP_001731966.1 |
| | A/S; AUG | MIC | | | | |
| 3348855 | CF; TE; CFZ; CP; AM; | AUG | 9.76E−023 | F60I | yhaI | YP_001731966.1 |
| | A/S; AUG | MIC | | | | |
| 3348858 | CF; TE; CFZ; CP; AM; | AUG | 9.76E−023 | L61I | yhaI | YP_001731966.1 |
| | A/S; AUG | MIC | | | | |
| 3348867 | CF; TE; CFZ; CP; AM; | AUG | 1.98E−022 | L64I | yhaI | YP_001731966.1 |
| | A/S; AUG | MIC | | | | |
| 3348874 | CF; TE; CFZ; CP; AM; | AUG | 2.51E−020 | −66? | yhaI | YP_001731966.1 |
| | A/S; AUG | MIC | | | | |
| 3348877 | CF; TE; CFZ; CP; AM; | AUG | 1.77E−020 | −67 | yhaI | YP_001731966.1 |
| | A/S; AUG | MIC | | | | |
| 3348879 | CF; T/S; TE; CFZ; CP; | AUG | 3.64E−022 | I68V | yhaI | YP_001731966.1 |
| | AM; A/S; AUG | MIC | | | | |
| 3348919 | CF; T/S; TE; A/S; AM; | AUG | 4.36E−019 | −81? | yhaI | YP_001731966.1 |
| | CFZ; AUG | MIC | | | | |
| 3348922 | CF; T/S; TE; A/S; AM; | AUG | 9.35E−020 | −82 | yhaI | YP_001731966.1 |
| | CFZ; AUG | MIC | | | | |
| 3348932 | CF; CFZ; AUG; AM; A/S | CF MIC | 7.48E−022 | F85L | yhaI | YP_001731966.1 |
| 3348939 | CF; T/S; TE; CFT; CFZ; | AUG | 1.21E−017 | F88V; F88I | yhaI | YP_001731966.1 |
| | CP; AZT; AM; A/S; AUG | MIC | | | | |
| 3348951 | CF; T/S; TE; CFZ; CP; | AUG | 6.56E−021 | L92F | yhaI | YP_001731966.1 |
| | AM; A/S; AUG | MIC | | | | |
| 3348969 | CF; TE; CFZ; CP; AM; | AUG | 2.58E−022 | F98V | yhaI | YP_001731966.1 |
| | A/S; AUG | MIC | | | | |
| 3348970 | CF; TE; CFZ; CP; AM; | AUG | 2.58E−022 | F98S | yhaI | YP_001731966.1 |
| | A/S; AUG | MIC | | | | |
| 3348975 | CF; TE; CFZ; CP; AM; | AUG | 2.58E−022 | T100S | yhaI | YP_001731966.1 |
| | A/S; AUG | MIC | | | | |
| 3348976 | CF; T/S; TE; CFZ; CP; | AUG | 1.86E−022 | T100I | yhaI | YP_001731966.1 |
| | LVX; AM; A/S; AUG | MIC | | | | |
| 3348985 | CF; T/S; TE; CFZ; CP; | AUG | 1.13E−022 | T103N | yhaI | YP_001731966.1 |
| | AM; A/S; AUG | MIC | | | | |
| 3364264 | CF; TE; AUG | AUG | 4.50E−014 | T28I | yhaC | YP_001731981.1 |
| | | MIC | | | | |
| 3364344 | CF; T/S; TE; AUG | AUG | 1.42E−014 | T55A | yhaC | YP_001731981.1 |
| | | MIC | | | | |
| 3364554 | CF; A/S; AM; AUG | AUG | 1.20E−017 | I125V | yhaC | YP_001731981.1 |
| | | MIC | | | | |
| 3364627 | CF; T/S; TE; AUG | AUG | 2.75E−013 | D149G | yhaC | YP_001731981.1 |
| | | MIC | | | | |
| 3364673 | T/S; TE; AUG | AUG | 1.63E−012 | D164E | yhaC | YP_001731981.1 |
| | | MIC | | | | |
| 3364674 | T/S; TE; AUG | AUG | 1.63E−012 | Y165H | yhaC | YP_001731981.1 |
| | | MIC | | | | |
| 3364675 | T/S; TE; AUG | AUG | 1.55E−012 | Y165F | yhaC | YP_001731981.1 |
| | | MIC | | | | |
| 3364731 | CF; AUG | AUG | 6.23E−012 | N184D | yhaC | YP_001731981.1 |
| | | MIC | | | | |
| 3364740 | AUG | AUG | 1.17E−010 | N187D | yhaC | YP_001731981.1 |
| | | MIC | | | | |
| 3364836 | AUG | AUG | 5.77E−012 | L219; L219I | yhaC | YP_001731981.1 |
| | | MIC | | | | |
| 3491646 | CF; TO; AUG | CF MIC | 2.30E−018 | SD126N | yhdP | YP_001732096.1 |
| 3727548 | CF; AM; AUG | AUG | 2.12E−015 | I271V | yhiJ | YP_001732321.1 |
| | | MIC | | | | |
| 3727549 | CF; AUG | CF MIC | 1.34E−014 | I270; I270M | yhiJ | YP_001732321.1 |
| 3728109 | CF; CFZ; AUG; AM; A/S | AUG | 8.41E−022 | D84N | yhiJ | YP_001732321.1 |
| | | MIC | | | | |
| 3888822 | CF; CP; CFZ; LVX; AM; | CF MIC | 5.08E−015 | N154K | htrL | YP_001732446.1 |
| | A/S; AUG | | | | | |
| 3888829 | CF; CP; CFZ; TO; AM; | CF MIC | 1.18E−014 | K152T | htrL | YP_001732446.1 |
| | A/S; LVX; AUG | | | | | |
| 3888830 | CF; CP; CFZ; TO; AM; | CF MIC | 1.12E−014 | K152* | htrL | YP_001732446.1 |
| | A/S; LVX; AUG | | | | | |
| 3888836 | CF; CP; CFZ; TO; AM; | CF MIC | 1.12E−014 | C150S | htrL | YP_001732446.1 |
| | A/S; LVX; AUG | | | | | |
| 3888838 | CF; CP; CFZ; TO; AM; | CF MIC | 1.27E−014 | Y149F | htrL | YP_001732446.1 |
| | A/S; LVX; AUG | | | | | |
| 4054212 | CF; CFZ; CRM; AM; A/S; | CF MIC | 1.48E−031 | E184; E184D | ilvY | YP_001732582.1 |
| | AUG | | | | | |
| 4240703 | CF | CF MIC | 4.18E−010 | −152? | frwC | YP_001732738.1 |
| 4349496 | CF; AUG | AUG | 1.81E−011 | N275D; N275H | yjbI | YP_001732815.1 |
| | | MIC | | | | |
| 4379996 | CF; AM; AUG | CF MIC | 7.21E−017 | I267V; I267F | yjcF | YP_001732841.1 |
| 4380134 | CF; AUG | AUG | 8.29E−013 | S221A | yjcF | YP_001732841.1 |
| | | MIC | | | | |
| 4380751 | CF; AM; AUG | AUG | 3.21E−015 | L15Q | yjcF | YP_001732841.1 |
| | | MIC | | | | |
| 4525092 | CF; T/S; CP; A/S; AM; | AUG | 2.20E−019 | A239D | yjfZ | YP_001732969.1 |
| | CFZ; AUG | MIC | | | | |
| 4525097 | CF; T/S; CP; A/S; AM; | AUG | 4.45E−019 | S237R | yjfZ | YP_001732969.1 |
| | CFZ; AUG | MIC | | | | |
| 4525109 | CF; T/S; CP; A/S; LVX; | AUG | 5.43E−020 | E233D | yjfZ | YP_001732969.1 |
| | AM; CFZ; AUG | MIC | | | | |
| 4575597 | CF; CP; GM; A/S; LVX; | AUG | 3.43E−014 | E110D; E110 | yjgL | YP_001733012.1 |
| | AM; AUG | MIC | | | | |
| 4575608 | TE; AM; AUG | AUG | 2.79E−012 | S114L | yjgL | YP_001733012.1 |
| | | MIC | | | | |
| 4575610 | CF; T/S; TE; CFZ; CP; | AUG | 2.73E−027 | P115S | yjgL | YP_001733012.1 |
| | LVX; AM; A/S; AUG | MIC | | | | |
| 4576732 | CF; A/S; AM; AUG | AUG | 3.86E−017 | M489L | yjgL | YP_001733012.1 |
| | | MIC | | | | |
| 4576737 | CF; A/S; AM; AUG | AUG | 2.31E−016 | N490K | yjgL | YP_001733012.1 |
| | | MIC | | | | |
| 4576912 | CF; T/S; CP; GM; CFZ; | AUG | 6.59E−024 | L549M | yjgL | YP_001733012.1 |
| | LVX; AM; A/S; AUG | MIC | | | | |

Besides the already described exchanges in GyrA and ParC, we discovered AA exchanges in YdjO associated with predicted resistance to different β-lactams (V121E, S120C, V118F, 1114V, K111E, and D112N). Likewise, for lactams we report AA exchanges in YcbS (E848Q, E848*), RhsC (R717Q, W492C), YcbQ (T86I), YagR (S274T), and YeaU (N293K). Finally, we discovered AA exchanges related to quinolones, tetracycline, and lactams in YhaL (altogether 23 different sites).

To check whether all 21 drugs show genome wide association with drug resistance we investigated the most significant non-synonymous AA exchange for each drug (p-value threshold<10⁻⁹). Of 21 tested drugs, only two (Imipenem, Meropenem) were not found to be associated with an AA exchange with such a low p-value. Interestingly, the S83L mutation in GyrA is the predominant exchange in 15 drugs. For the drugs Ciprofloxacin and Levofloxacin, of which GyrA is a target, the p-values were however much lower than the p-values for this mutation in association with the remaining 13 drugs (>10⁻⁶² vs. <10⁻²⁰⁹). In addition, we observed again a significant decrease in sensitivity and/or PPV in these cases: either the sensitivity or PPV is below 55% for drugs, of which GyrA is not the target, demonstrating that these measures are effective for separating mutations in true targets from others.

Analogously, we analyzed the 1,176 K. pneumoniae isolates (p-value threshold<10⁻⁹) by mapping the generated NGS reads against the reference strain MGH 78578 (NC_009648). We discovered 1,456,074 genomic positions that passed the quality filter and where at least one sample had a non-reference base. After correlating the genetic variation data with the AST data, 40,896 unique genomic positions remained that coded for a non-synonymous variant and were associated with at least one drug. The highest significance (before FDR-adjustment) was reached for the AA exchange S80I in ParC for the quinolones Ciprofloxacin (p=10⁻¹⁶⁰) and Levofloxacin (p=10⁻¹⁴². The second target of these two drugs, GyrA, shows the lowest p-values for AA exchange Y83N (p=10⁻¹¹² for Ciprofloxacin and p=10⁻¹¹¹ for Levofloxacin). In addition, we detected two positions (G234S, E235K) in KPN_01607, also known as β-lactamase SHV-11, significantly associated with all tested drugs, but especially reaching low p-values for the drug class lactams. Here, Cefotaxime reaches the lowest p-value (10⁻¹²²) for G234S and Aztreonam the lowest (10⁻¹²¹) for E235K.

TABLE 15
Identified mutations in known targets

| Drug | Known Target | identified mutations in our analysis |
|---|---|---|
| Ciprofloxacin | GyrA | S83L, D87N, D87Y, D678E, E574D |
| Levofloxacin | GyrA | S83L, D87N, D87Y, D678E, E574D |
| Ciprofloxacin | ParC | S80I, E84G, E84V, E84A, A192V, |
| | | Q481H, A471G, T718A, Q198H |
| Levofloxacin | ParC | S80I, E84G, E84V, E84A, A192V, |
| | | Q481H, A471G, T718A, Q198H |
| Cefalotin | AmpC | K40R, I300V, T335I, A210P, |
| | | Q196H, A236T, R248C |
| Sulfamethoxazole | FolC | A319T, R88C, G217S |
| Cefazolin | MrcB | D839E, QQQP815Q, R556C |
| Cefazolin | PbpC | L357V, V348A, A15T, A217V, |
| | | Q495L, V768F, A701E, K766R, |
| | | K766T, T764S, T764A, R602L, |
| | | E446G, R669H, A202T |
| Ceftazidime | PbpG | A28V |

In a next step, we assessed whether an overlap of mutations in functionally similar proteins of the two genera exists. Interestingly, when considering the proteins that were associated significantly with at least one drug, we found an overlap of 1,746 proteins (same official name and more than 80 percent positives in BLAST in pairwise comparison) that are affected in E. coli as well as in K. pneumoniae. Extending the analysis to the exact AA exchanges in these proteins, we still detect an overlap of 55 mutated positions that are equal in both organisms. Amongst those common mutations are for example D87N and D87Y in GyrA, and S80I and S80R in ParC. Furthermore, many of these proteins are associated with diverse metabolic pathways, e.g. the thiamine metabolism (Dxs, ThiC, ThiE, ThiM) or the purine metabolism (CysD, PurH, PurK, PurL YjjG). A complete list of the proteins and the identical AA exchanges can be found in Table 15.

### Mutations in Known Drug Targets

In the previous section we already reported highly significant mutations in drug targets. Beyond these, we systematically searched for AA exchanges in other known drug targets. For E. coli such AA exchanges significantly associated with drugs were detected in nine cases. For K. pneumoniae we respectively discovered AA exchanges in 10 drug targets. In addition to the already described overlap in ParC and GyrA, exchanges in the proteins FolC, MrcB, and PbpC overlapped in both genera. The complete list of affected sites is provided in Table 16.

TABLE 16
Overlapping Mutations in E. Coli and Klebsiella pneumonia

| Gene | Amino Acid |
|---|---|
| Name | Exchange |
| aspS | D382E |
| birA | Q113H |
| cysD | D232N |
| dapB | N87K |
| dxs | A541T |
| eutA | A210V |
| fadA | V387I |
| fdx | S66T |
| fhuB | G448V |
| fhuC | A122V |
| fhuD | D76E |
| fmt | V30I |
| gudP | A448V |
| gyrA | D87N; D87Y |
| helD | E671D |
| hrpB | A413T |
| hrpB | V240A |
| ilvA | D401E |
| kdpD | E376D |
| ldcA | R167Q |
| lplA | A279T |
| menB | T31A |
| metH | E1124; E1124D |
| mukB | S1015N |
| parC | S80I |
| parC | S80R |
| pbpC | H37Q |
| purH | T366I |
| purK | N137D |
| purL | D615E |
| queF | K126E |
| rhaA | S406N |
| rhaB | T407A |
| rplO | K39N |
| srlD | M54T |
| thiC | H193R |
| thiE | A121E |
| thiE | R43Q |
| thiM | A122T |
| trpC | L378F |
| udp | I147M |
| uxaA | E236A |
| ybiB | G35S |
| ybiU | M419I |
| ydfI | A146V |
| ydgA | F416L |
| yecA | I195V |
| yehT | A106V |
| yfcN | I39V |
| yheN | Q49H |
| yhgF | E737D |
| yhhQ | R138H |
| yhjE | I323V |
| yjjG | A57V |
| ynfA | T84S |

Most affected genes and multi-drug resistant sites in E. coli

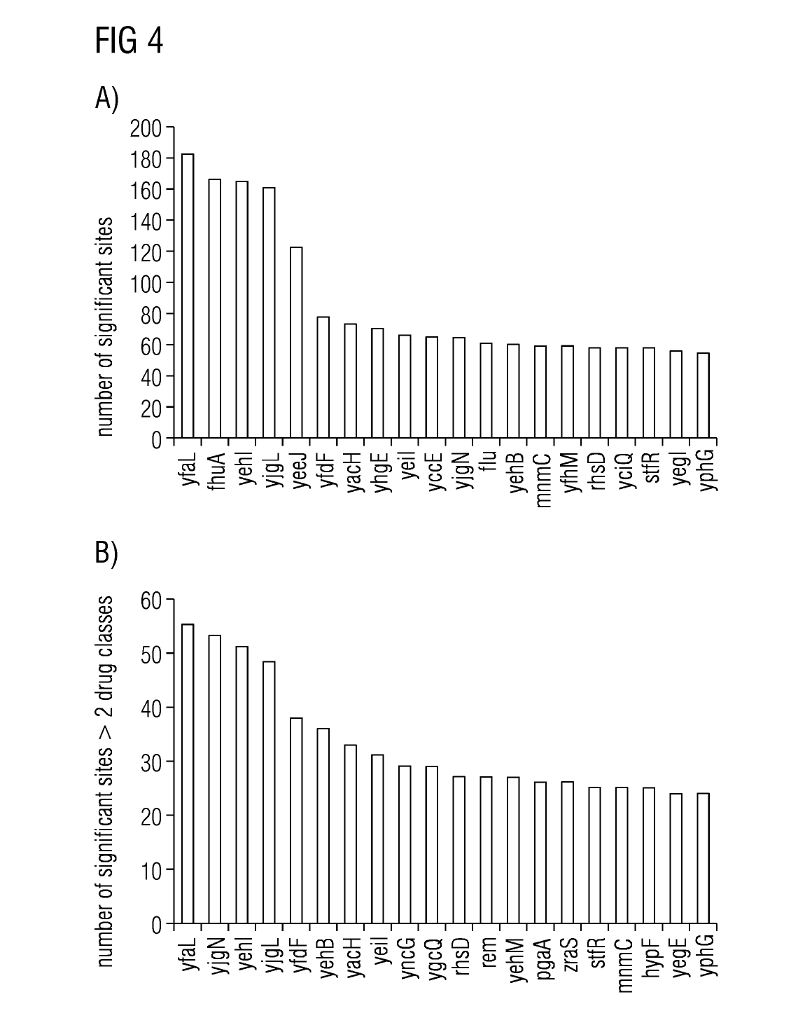

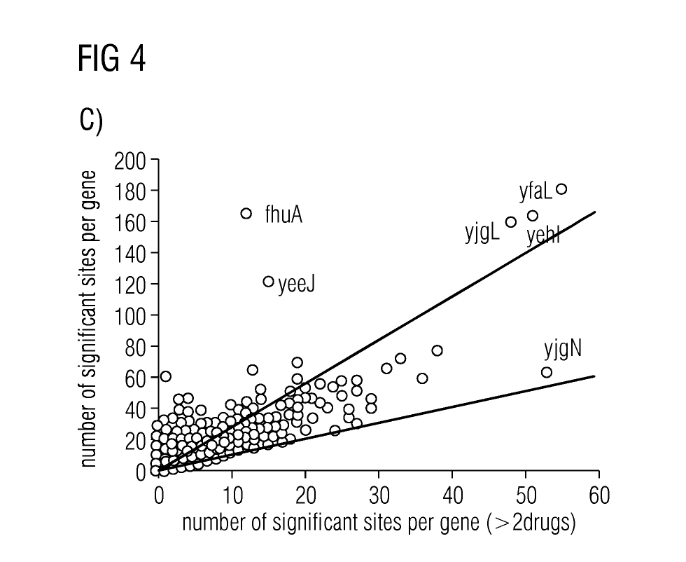

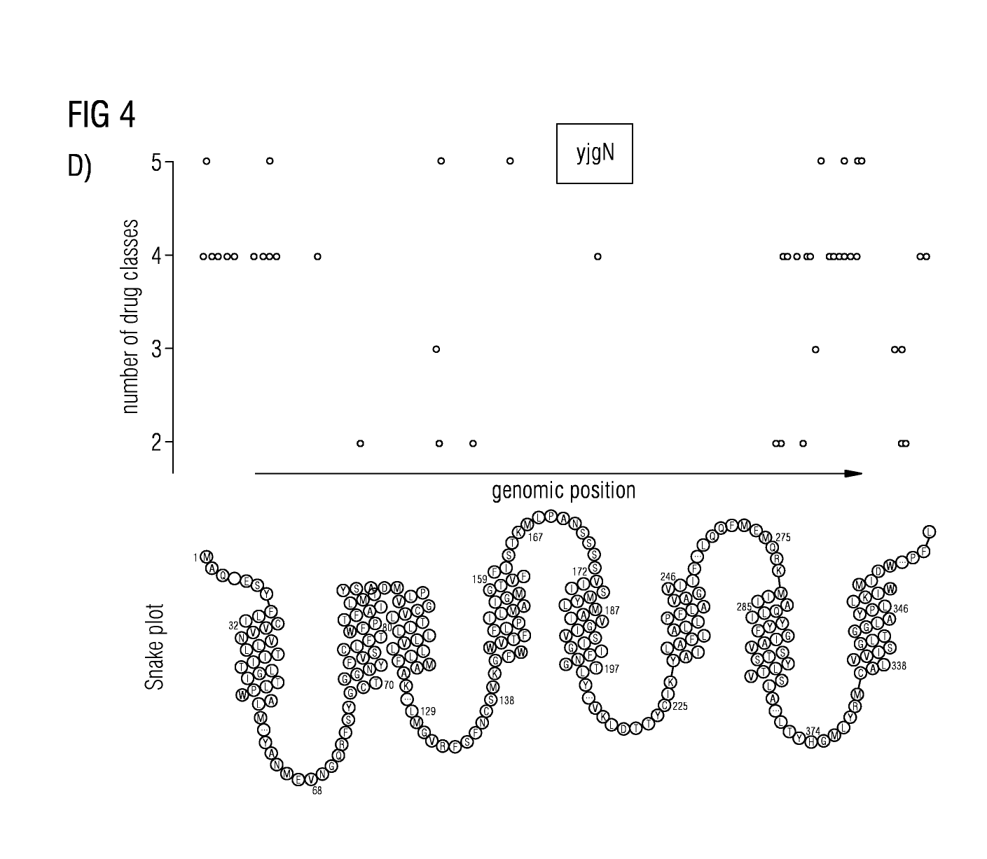

Next, we analyzed the distribution of the non-synonymous variants in the genomes, showing that the mutations are not uniformly distributed across E. coli genes, details thereof being shown in FIG. 4: for example yfaL, fhuA, yehI, yjgL, and yeeJ carry over 120 non-synonymous variants per gene (FIG. 4A); in yfaL, as many as 182 significant exchanges were discovered. In order to discover sites that are relevant for multi-drug resistance, we calculated the number of AA exchanges significant in association with at least 3 drug classes (FIG. 4B) and plotted the respective site counts for each gene in FIG. 4C. On average, 35% of all significant sites were associated with at least three drugs. While three genes, yfaL, yehI, and yjgL, had the highest number of AA exchanges, yjgN had a substantially increased number of sites associated with multi-drug resistance (53 of 64 sites, 83%), while yeeJ (15 of 122 sites, 12%) and fhuA (12 of 166 sites, 7%) carry fewer sites relevant for multiple drug classes than expected. In yjgN, the positions significantly associated with multiple drug classes were concentrated in the terminal regions of the gene (FIG. 4D). In summary, FIG. 4 shows: Panel A: bar chart of E. coli genes with highest number of significant sites. Panel B. bar chart detailing the genes with highest number of sites correlated to at least 3 drugs. Panel C. Scatter plot showing for each gene the number of significant sites correlated with at least 3 drugs as function of total number of significant sites in the gene. Panel D. Along gene plot for yjgN. The significant sites along the genetic sequence are presented as dots, the y-axis shows the number of drug classes significant for the respective site. Below, a so called snake plot of the trans-membrane protein is shown, the affected amino acids are colored.

Analyzing Combinations of Mutations to Predict Resistance with Decision Trees

As previously mentioned on the example of gyrA and parC, a single mutation is often not sufficient to confer resistance for a certain type of drug and multiple mutations can have a cumulative effect (see FIG. 3). Additionally, the accuracy, specificity, and sensitivity for the single sites may overestimate the actual performance since we selected the optimal thresholds on the complete data set. Therefore, we applied decision tree learning on resistances defined according to EUCAST MIC breakpoints and evaluated the approach relying on combinations of mutations using 10 repetitions of 5-fold cross validation.

Due to the relative low number of resistant E. coli samples according to the EUCAST guidelines for some drugs in the data set we here focus on K. pneumoniae. As expected we did not observe a significantly improved accuracy of the prediction in general due to the above mentioned optimal scenario for single mutations and the class imbalance. While the median specificity for the single mutations was 98.9%, the sensitivity was just 61.3%. For the decision trees, the specificity was still 94%, however the sensitivity increased significantly to 75%. Classification accuracy was in the range of 76.8% (Ampicillin/Sulbactam) to 96.1% (Meropenem). Remarkably, all drugs besides Ampicillin/Sulbactam reached accuracy above 80%. For 6 drugs even the 90% threshold was exceeded. The tree size varied between 1 up to 9 mutations (Supplemental FIG. 1) and altogether just 14 mutations in 13 different genes were required to build all trees.

### Analysis of Gene Dosage Effects

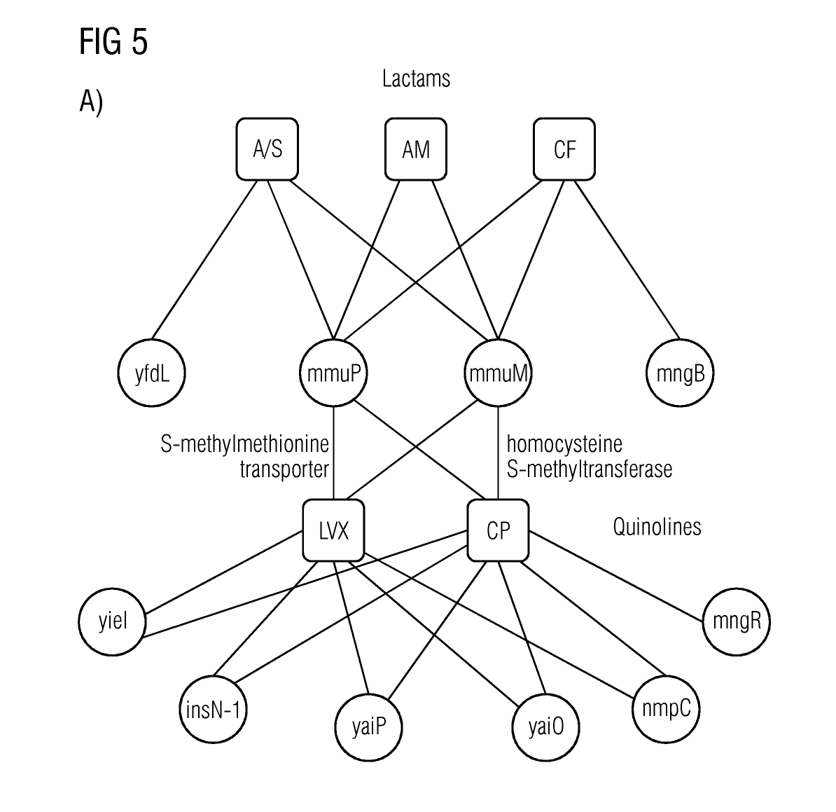

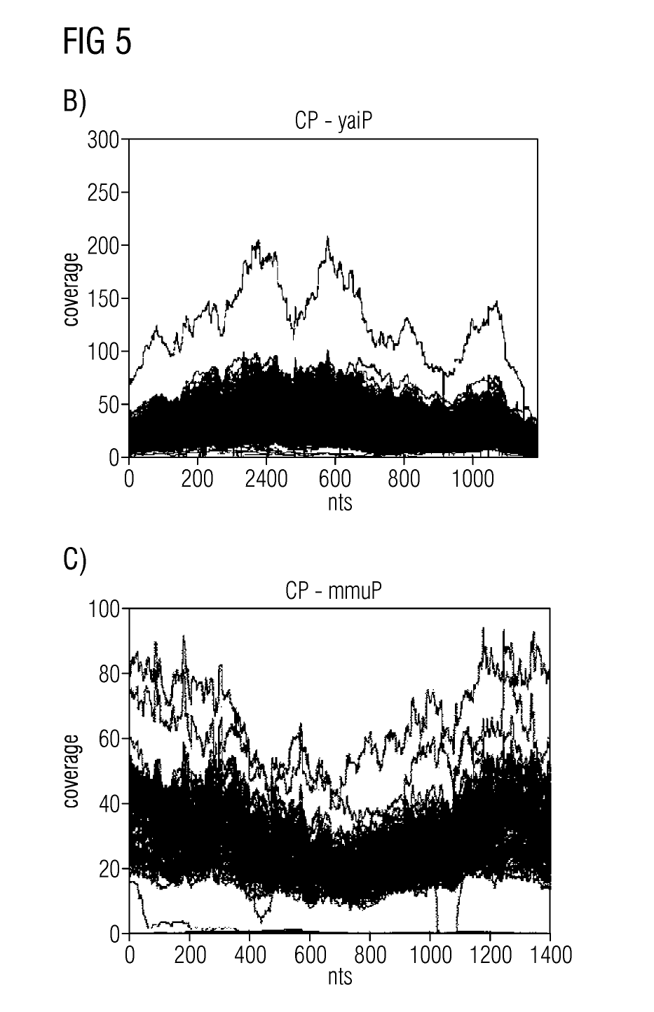

A potential reason for drug resistance is gene duplication or deletion, which can be observed in our dataset by inspecting the read coverage of different genes in the groups of resistant and susceptible isolates. To estimate the difference in coverage we calculated AUC values for the normalized median coverage per gene in the two groups. Altogether we discovered 23 cases of abnormal differences in gene coverage of 10 genes between resistant and susceptible E. coli bacteria resulting in an AUC>0.75 (FIG. 4A). We report connections for three β-lactams and two quinolones. Central genes are mmuP and mmuM, encoding for a putative S-methylmethionine transporter and a homocysteine S-methyltransferase, respectively, for which the coverage is substantially higher in bacteria resistant to all 5 drugs. In strains resistant to Levofloxacin and Ciprofloxacin, the inner membrane protein YieI and InsN-1, a regulator of insertion element, were likewise higher abundant. In contrast, genes encoding glucosyltransferases YaiP, YaiO, outer membrane protein NmpC and DNA-binding transcriptional repressor MngR were less covered in strains resistant to these drugs. Details are shown in FIG. 5, with FIG. 5 showing: Panel A: network diagram showing drugs as rectangles and E. coli genes with higher or lower coverage if resistance for the respective drug is shown as circles. Panel B and C: two example along-chromosome plots.

FIGS. 5B and 5C show an example coverage plot for the lower abundant covered yaiP and the higher abundant covered mmuP in strains resistant to Ciprofloxacin. Best diagnostic accuracy was reached for Ciprofloxacin and the gene mmuP, with an AUC value of 0.923, demonstrating that this quantitative information allows for accurate separation between resistant and susceptible strains.

For K. pneumoniae we found 216 cases of abnormal differences in gene coverage resulting in an AUC>0.75. We found drug/gene dosage combinations for all drugs except the drug combination Trimethoprim Sulfamethoxazole and 32 different genes. The best AUC value of 0.90 was observed for the drug Meropenem and the gene rm1C. The drugs Ertapenem, Imipenem, and Amoxicillin Clavulanate reached for the same gene an almost as high AUC value of 0.89. In total this gene is associated with the most number of drugs (17). The coverage of this gene is lower in bacteria resistant to those drugs. Additionally, we found the gene KPN_01607 associated with 14 drugs, where Aztreonam has the best AUC value of 0.85. For this gene, the coverage is generally higher in bacteria resistant to those drugs. An overview of the AUC values for E. coli and K. pneumoniae can be found in Tables 17 and 18.

TABLE 17
Gene Dosage/AUC values for E. coli

| DRUG | RANGE | BEST_BREAK | BEST_AUC | MEDIAN_LOW_RES | MEDIAN_HIGH_RES |
|---|---|---|---|---|---|
| CP MIC | mmuP: 248653-250056 | 0.5 | 0.923223957 | 20.52644867 | 31.82392132 |
| LVX MIC | mmuP: 248653-250056 | 0.5 | 0.920527671 | 20.47389478 | 31.78368944 |
| CP MIC | mmuM: 250043-250975 | 0.5 | 0.914622491 | 18.55525678 | 28.36308236 |
| LVX MIC | mmuM: 250043-250975 | 0.5 | 0.904118404 | 18.59265459 | 28.32466583 |
| AM MIC | mmuP: 248653-250056 | 8 | 0.822433611 | 21.73847687 | 31.31254354 |
| A/S MIC | mmuP: 248653-250056 | 8 | 0.81234027 | 21.73847687 | 31.37059415 |
| CF MIC | mmuM: 250043-250975 | 16 | 0.808572618 | 19.88839373 | 27.78921807 |
| LVX MIC | yaiO: 318624-319397 | 8 | 0.806781264 | 20.96897261 | 15.77018012 |
| CP MIC | yaiO: 318624-319397 | 8 | 0.806104208 | 20.97901322 | 15.77018012 |
| AM MIC | mmuM: 250043-250975 | 8 | 0.805390408 | 19.88839373 | 27.78921807 |
| CF MIC | mmuP: 248653-250056 | 16 | 0.802746567 | 20.85973599 | 30.75984192 |
| A/S MIC | mmuM: 250043-250975 | 8 | 0.788243885 | 19.88839373 | 27.82221604 |
| LVX MIC | yaiP: 321294-322490 | 8 | 0.786212152 | 25.97043332 | 21.2670006 |
| CP MIC | yaiP: 321294-322490 | 8 | 0.783618797 | 25.98139883 | 21.35135079 |
| CP MIC | nmpC: 513084-515380 | 8 | 0.778358209 | 1.703782828 | 0.474851188 |
| LVX MIC | nmpC: 513084-515380 | 8 | 0.778358209 | 1.703782828 | 0.474851188 |
| A/S MIC | yfdL: 2561331-2561849 | 4 | 0.767783657 | 3.061785609 | 1.020546319 |
| CP MIC | insN-1: 243570-243974 | 8.1 | 0.765352641 | 9.99083341 | 38.16571264 |
| CP MIC | yieI: 3993113-3993580 | 0.25 | 0.761230853 | 3.31276362 | 16.47983536 |
| CP MIC | mngR: 816968-817690 | 8.1 | 0.761213779 | 13.87434469 | 10.564168 |
| LVX MIC | insN-1: 243570-243974 | 8 | 0.758469449 | 9.938252806 | 36.52230829 |
| CF MIC | mngB: 819793-822426 | 16 | 0.752116402 | 17.21010306 | 9.363371994 |
| LVX MIC | yieI: 3993113-3993580 | 0.5 | 0.752078232 | 3.308373493 | 16.38090416 |

TABLE 18
Gene Dosage/AUC values for K. pneumonia

| DRUG | RANGE | BEST_BREAK | BEST_AUC | MEDIAN_LOW_RES | MEDIAN_HIGH_RES |
|---|---|---|---|---|---|
| MER MIC | rmlC: 2723257-2723811 | 8 | 0.89568662 | 13.40282052 | 8.272842006 |
| IMP MIC | KPN_02272: 2484450-2485832 | 2 | 0.893311711 | 11.22251065 | 1.72851308 |
| AUG MIC | rmlC: 2723257-2723811 | 64.1 | 0.892880722 | 13.29540108 | 8.272842006 |
| IMP MIC | rmlC: 2723257-2723811 | 4 | 0.892082839 | 13.40282052 | 8.272842006 |
| MER MIC | KPN_02272: 2484450-2485832 | 2 | 0.890520564 | 11.22421273 | 1.746964126 |
| ETP MIC | KPN_02272: 2484450-2485832 | 1 | 0.889760626 | 11.33348839 | 1.749211059 |
| ETP MIC | rmlC: 2723257-2723811 | 2 | 0.8875 | 13.43527281 | 8.343040081 |
| P/T MIC | rmlC: 2723257-2723811 | 256.1 | 0.867509409 | 13.44561317 | 8.474906092 |
| CPE MIC | KPN_02272: 2484450-2485832 | 64.1 | 0.865224179 | 11.03101065 | 1.751457992 |
| AUG MIC | KPN_02272: 2484450-2485832 | 64 | 0.858498024 | 11.17725495 | 1.767138965 |
| CFT MIC | KPN_02272: 2484450-2485832 | 128.1 | 0.8580088 | 11.15457021 | 1.767651622 |
| LVX MIC | KPN_02272: 2484450-2485832 | 16.1 | 0.847840148 | 11.03101065 | 1.815254308 |
| AZT MIC | KPN_01607: 1784144-1785004 | 64.1 | 0.845885671 | 11.91976074 | 32.72807977 |
| CAX MIC | KPN_02272: 2484450-2485832 | 128.1 | 0.839517181 | 11.22421273 | 1.848467103 |
| CP MIC | KPN_04541: 4973659-4974345 | 8 | 0.835659904 | 13.12618136 | 1.235897636 |
| LVX MIC | rmlC: 2723257-2723811 | 16.1 | 0.834716599 | 13.42852053 | 8.648349078 |
| CPE MIC | rmlC: 2723257-2723811 | 64.1 | 0.834115966 | 13.29540108 | 8.550440162 |
| CFT MIC | rmlC: 2723257-2723811 | 128.1 | 0.832094953 | 13.42852053 | 8.516219091 |
| CF MIC | KPN_01607: 1784144-1785004 | 64.1 | 0.831273888 | 11.83575783 | 30.59745795 |
| P/T MIC | KPN_01607: 1784144-1785004 | 128 | 0.831059554 | 11.97628559 | 31.83680721 |
| LVX MIC | KPN_04541: 4973659-4974345 | 8 | 0.830951605 | 13.12004558 | 1.253706365 |
| CP MIC | KPN_03336: 3652949-3653470 | 8.1 | 0.830140187 | 11.03109887 | 26.42333762 |
| AZT MIC | ygbI: 1783401-1784123 | 64.1 | 0.829964951 | 14.09112956 | 37.31434545 |
| CFZ MIC | KPN_01607: 1784144-1785004 | 32.1 | 0.829950143 | 11.83904928 | 30.48932358 |
| CAX MIC | rmlC: 2723257-2723811 | 128 | 0.829056707 | 13.44561317 | 8.648349078 |
| CAZ MIC | KPN_01607: 1784144-1785004 | 64.1 | 0.828960959 | 11.93090685 | 31.58020077 |
| CRM MIC | rmlC: 2723257-2723811 | 64.1 | 0.828061315 | 13.50276659 | 8.712417691 |
| CP MIC | rmlC: 2723257-2723811 | 8.1 | 0.818556701 | 13.47075713 | 8.78669993 |
| CAX MIC | KPN_01607: 1784144-1785004 | 4 | 0.818389606 | 11.87098062 | 30.3872813 |
| CFT MIC | KPN_01607: 1784144-1785004 | 2 | 0.818292595 | 11.87322455 | 30.42138682 |
| AZT MIC | rmlC: 2723257-2723811 | 64.1 | 0.816740088 | 13.4959011 | 8.832904621 |
| CP MIC | KPN_02272: 2484450-2485832 | 8.1 | 0.815759218 | 11.13188548 | 1.911839013 |
| AZT MIC | KPN_02272: 2484450-2485832 | 64.1 | 0.814249741 | 11.91707435 | 2.104436573 |
| CPE MIC | KPN_01607: 1784144-1785004 | 2 | 0.813449623 | 11.90853472 | 30.45633912 |
| CAX MIC | KPN_04541: 4973659-4974345 | 64 | 0.813406165 | 12.65078139 | 1.253706365 |
| CF MIC | ygbI: 1783401-1784123 | 64.1 | 0.812061697 | 13.9758737 | 35.17900985 |
| CAZ MIC | ygbI: 1783401-1784123 | 64.1 | 0.811121897 | 14.16929473 | 35.87435366 |
| CFZ MIC | ygbI: 1783401-1784123 | 32.1 | 0.810320337 | 13.98579807 | 35.17900985 |
| AUG MIC | rmlC: 2715683-2716237 | 64.1 | 0.808350101 | 5.782817287 | 12.25321066 |
| P/T MIC | ygbI: 1783401-1784123 | 128 | 0.807941775 | 14.1838383 | 35.58743149 |
| AUG MIC | KPN_01784: 1966562-1967182 | 32 | 0.807924725 | 17.87664169 | 0.643613711 |
| CRM MIC | KPN_02272: 2484450-2485832 | 64.1 | 0.807799443 | 11.33348839 | 1.999569508 |
| CPE MIC | KPN_04541: 4973659-4974345 | 4 | 0.804937919 | 13.1139098 | 1.476953034 |
| AUG MIC | KPN_04541: 4973659-4974345 | 32 | 0.804920025 | 11.30622679 | 1.137186158 |
| CRM MIC | KPN_04541: 4973659-4974345 | 64.1 | 0.804857557 | 11.50624406 | 1.215886481 |
| P/T MIC | ygbJ: 1782199-1783101 | 128 | 0.804644984 | 12.2629073 | 30.24050414 |
| A/S MIC | rmlC: 2723257-2723811 | 64.1 | 0.804008715 | 13.44561317 | 8.916947739 |
| CAZ MIC | KPN_02272: 2484450-2485832 | 64.1 | 0.803300392 | 11.79582178 | 2.120499505 |
| CAX MIC | ygbI: 1783401-1784123 | 1 | 0.802474553 | 13.98579807 | 34.70442867 |
| CFT MIC | ygbI: 1783401-1784123 | 2 | 0.801616933 | 14.0033996 | 35.08360594 |
| AZT MIC | KPN_01784: 1966562-1967182 | 64.1 | 0.801256233 | 17.99164875 | 0.651206715 |
| CAZ MIC | KPN_01784: 1966562-1967182 | 64.1 | 0.798468554 | 17.99545075 | 0.735867662 |
| CP MIC | KPN_01784: 1966562-1967182 | 8 | 0.798148148 | 17.89379021 | 0.619814909 |
| AM MIC | KPN_01607: 1784144-1785004 | 128.1 | 0.797712248 | 11.72073654 | 26.6117317 |
| CPE MIC | ygbI: 1783401-1784123 | 2 | 0.796981789 | 14.06017543 | 35.0664591 |
| A/S MIC | KPN_01607: 1784144-1785004 | 64.1 | 0.796930803 | 11.99343966 | 30.09252392 |
| CFT MIC | KPN_01784: 1966562-1967182 | 64 | 0.796141999 | 17.87664169 | 0.621681001 |
| A/S MIC | ygbJ: 1782199-1783101 | 64.1 | 0.79545801 | 12.25998772 | 29.74457325 |
| TO MIC | KPN_01607: 1784144-1785004 | 16 | 0.794383461 | 11.98391794 | 30.45591573 |
| IMP MIC | rmlC: 2715683-2716237 | 8 | 0.793880035 | 5.820919849 | 11.91623279 |
| LVX MIC | KPN_01784: 1966562-1967182 | 8 | 0.793541677 | 17.91093874 | 0.635529129 |
| MER MIC | rmlC: 2715683-2716237 | 16 | 0.793143657 | 5.843850019 | 11.93620101 |
| P/T MIC | KPN_01784: 1966562-1967182 | 128 | 0.79253037 | 17.94616152 | 0.643613711 |
| AZT MIC | KPN_04541: 4973659-4974345 | 64 | 0.791641389 | 13.18891293 | 1.900944326 |
| TO MIC | KPN_04541: 4973659-4974345 | 16 | 0.790992556 | 11.23590819 | 1.461109259 |
| LVX MIC | KPN_03336: 3652949-3653470 | 8 | 0.789430582 | 11.07003498 | 25.97210426 |
| CFT MIC | KPN_04541: 4973659-4974345 | 32 | 0.78910942 | 12.28426879 | 1.461109259 |
| CRM MIC | KPN_00957: 1081235-1081342 | 32 | 0.788235294 | 12.78864958 | 22.69865562 |
| TO MIC | KPN_01784: 1966562-1967182 | 16 | 0.788128307 | 17.95391995 | 0.654073833 |
| TE MIC | KPN_04868: 3814427-3815335 | 64.1 | 0.787964876 | 4.525426107 | 14.04476546 |
| P/T MIC | KPN_02272: 2484450-2485832 | 256.1 | 0.787303266 | 11.22251065 | 2.039857307 |
| CAX MIC | KPN_03336: 3652949-3653470 | 128 | 0.787200306 | 11.21785008 | 26.12424811 |
| CFT MIC | KPN_00957: 1081235-1081342 | 8 | 0.787068005 | 12.44073167 | 22.64953743 |
| CAZ MIC | rmlC: 2723257-2723811 | 64.1 | 0.786884532 | 13.42935845 | 8.929866606 |
| CPE MIC | KPN_03336: 3652949-3653470 | 8 | 0.786687943 | 11.19265939 | 25.60940404 |
| CRM MIC | KPN_01607: 1784144-1785004 | 64 | 0.786592102 | 11.96508781 | 30.45591573 |
| CFZ MIC | rmlC: 2723257-2723811 | 32.1 | 0.786353643 | 13.93854496 | 9.164263895 |
| A/S MIC | KPN_01784: 1966562-1967182 | 64.1 | 0.786127646 | 17.95391995 | 0.675638197 |
| CAX MIC | KPN_00957: 1081235-1081342 | 16 | 0.785793256 | 12.84408698 | 22.74777381 |
| LVX MIC | rmlC: 2715683-2716237 | 16.1 | 0.785699589 | 5.703503092 | 11.78451923 |
| P/T MIC | ygbK: 1780922-1782187 | 128 | 0.78549987 | 12.43522837 | 28.57180222 |
| CPE MIC | KPN_00957: 1081235-1081342 | 2 | 0.783195592 | 12.89952438 | 23.01649393 |
| CAZ MIC | KPN_01762: 1944660-1945901 | 64.1 | 0.783184653 | 14.68598492 | 12.20397515 |
| ETP MIC | KPN_03336: 3652949-3653470 | 0.25 | 0.782849072 | 11.21858903 | 26.17310699 |
| CAZ MIC | KPN_00957: 1081235-1081342 | 64 | 0.782417582 | 12.84408698 | 22.74777381 |
| AUG MIC | KPN_01607: 1784144-1785004 | 8 | 0.781945351 | 11.80849185 | 26.49563733 |
| CRM MIC | KPN_01784: 1966562-1967182 | 64.1 | 0.780750543 | 17.92855013 | 0.735232226 |
| CPE MIC | KPN_01784: 1966562-1967182 | 4 | 0.780702996 | 17.94616152 | 0.686742437 |
| CAZ MIC | KPN_04541: 4973659-4974345 | 64.1 | 0.780642008 | 12.28426879 | 1.772016957 |
| CAX MIC | KPN_01784: 1966562-1967182 | 64 | 0.780397544 | 17.87507462 | 0.651206715 |
| TO MIC | rmlC: 2723257-2723811 | 16 | 0.78019527 | 13.44044299 | 8.960257858 |
| P/T MIC | KPN_01790: 1972135-1973631 | 128 | 0.780107599 | 12.96633239 | 10.70048935 |
| AZT MIC | KPN_02270: 2483617-2483943 | 64 | 0.779995637 | 13.23289566 | 7.277468931 |
| AM MIC | ygbI: 1783401-1784123 | 128.1 | 0.779602341 | 13.89270538 | 31.06241558 |
| CF MIC | KPN_00957: 1081235-1081342 | 8 | 0.77948718 | 11.38737121 | 21.45006033 |
| CF MIC | rmlC: 2723257-2723811 | 64.1 | 0.779210157 | 13.9291492 | 9.265493449 |
| A/S MIC | ygbK: 1780922-1782187 | 64.1 | 0.77850594 | 12.43242533 | 27.91657779 |
| CPE MIC | KPN_04540: 4971080-4973572 | 8 | 0.778318187 | 7.70316093 | 2.099521426 |
| CFT MIC | KPN_02270: 2483617-2483943 | 16 | 0.778095238 | 13.12302421 | 7.277468931 |
| TO MIC | KPN_02272: 2484450-2485832 | 16 | 0.778040718 | 11.42199291 | 2.119236736 |
| CAX MIC | KPN_02270: 2483617-2483943 | 16 | 0.777221527 | 13.17795993 | 7.341302223 |
| P/T MIC | KPN_01776: 1957110-1958672 | 128 | 0.777036566 | 14.81289751 | 12.43362001 |
| CAZ MIC | KPN_01776: 1957110-1958672 | 64.1 | 0.777027027 | 14.85321289 | 12.74383855 |
| A/S MIC | ygbI: 1783401-1784123 | 64.1 | 0.776514612 | 14.19415214 | 34.27339195 |
| AZT MIC | KPN_01793: 1975788-1977488 | 16 | 0.776334776 | 19.68538242 | 17.32769083 |
| AZT MIC | KPN_01762: 1944660-1945901 | 64.1 | 0.776223445 | 14.68109398 | 12.2997784 |
| CP MIC | KPN_01182: 1331512-1332783 | 8.1 | 0.77597715 | 33.90059737 | 117.9098654 |
| TO MIC | ygbI: 1783401-1784123 | 16 | 0.775686356 | 14.18811499 | 34.65371696 |
| CAZ MIC | KPN_01767: 1948966-1949805 | 64.1 | 0.775193664 | 14.40162572 | 11.88236254 |
| P/T MIC | KPN_03336: 3652949-3653470 | 256 | 0.774649889 | 11.21711113 | 25.5773437 |
| AZT MIC | KPN_01767: 1948966-1949805 | 64.1 | 0.774573965 | 14.40487756 | 11.89608685 |
| TO MIC | KPN_01767: 1948966-1949805 | 16 | 0.774396368 | 14.39134677 | 11.95643648 |
| CAZ MIC | KPN_01790: 1972135-1973631 | 64.1 | 0.773403035 | 12.99882265 | 10.81390631 |
| A/S MIC | KPN_00957: 1081235-1081342 | 32 | 0.772807018 | 12.09281376 | 22.46187896 |
| AZT MIC | KPN_00957: 1081235-1081342 | 4 | 0.772017837 | 12.84408698 | 22.62765659 |
| AZT MIC | KPN_01790: 1972135-1973631 | 64.1 | 0.771785261 | 13.01072986 | 10.81390631 |
| P/T MIC | KPN_01772: 1953630-1954052 | 128 | 0.771423113 | 17.39355932 | 13.9739268 |
| LVX MIC | KPN_01182: 1331512-1332783 | 16 | 0.771390545 | 34.70857051 | 117.7840314 |
| CAZ MIC | KPN_01793: 1975788-1977488 | 16 | 0.771284271 | 19.66043748 | 17.32769083 |
| P/T MIC | KPN_00957: 1081235-1081342 | 8 | 0.77124183 | 11.38737121 | 22.14617907 |
| CP MIC | KPN_00957: 1081235-1081342 | 0.25 | 0.770175439 | 12.78864958 | 22.55372751 |
| AZT MIC | KPN_01776: 1957110-1958672 | 64 | 0.770026677 | 14.87410862 | 12.81269447 |
| CFT MIC | KPN_00596: 673027-673989 | 8 | 0.769586814 | 23.10041869 | 20.5721784 |
| MER MIC | hsdM: 1081394-1083421 | 32 | 0.769230769 | 24.48889718 | 34.03527833 |
| CRM MIC | ygbI: 1783401-1784123 | 64 | 0.76903009 | 14.17434013 | 34.70442867 |
| CFZ MIC | KPN_00957: 1081235-1081342 | 32.1 | 0.76826484 | 12.44073167 | 22.47979844 |
| CAZ MIC | KPN_02270: 2483617-2483943 | 32 | 0.768005681 | 13.24351239 | 7.569358983 |
| P/T MIC | KPN_04541: 4973659-4974345 | 64 | 0.767556671 | 11.76260506 | 2.094996145 |
| AUG MIC | ygbI: 1783401-1784123 | 8 | 0.767471746 | 13.94307606 | 30.97795923 |
| CP MIC | KPN_04540: 4971080-4973572 | 8 | 0.767266272 | 8.044954992 | 2.166850367 |
| TO MIC | KPN_01776: 1957110-1958672 | 16 | 0.767064217 | 14.78896152 | 12.52829143 |
| GM MIC | KPN_01607: 1784144-1785004 | 1 | 0.765133267 | 11.92485348 | 29.2901078 |
| LVX MIC | KPN_04540: 4971080-4973572 | 8 | 0.765095256 | 8.044954992 | 2.166850367 |
| CAZ MIC | KPN_01765: 1947291-1947956 | 64.1 | 0.765013618 | 12.24952635 | 9.807472313 |
| CAZ MIC | KPN_01766: 1947966-1948751 | 64.1 | 0.764918657 | 14.82038829 | 12.42014088 |
| CPE MIC | KPN_01767: 1948966-1949805 | 4 | 0.764909475 | 14.38608928 | 11.88236254 |
| TO MIC | KPN_01790: 1972135-1973631 | 16 | 0.76446281 | 12.98085477 | 10.96228877 |
| P/T MIC | KPN_01767: 1948966-1949805 | 128 | 0.76401929 | 14.35342113 | 11.8756885 |
| ETP MIC | rmlC: 2715683-2716237 | 16 | 0.764016471 | 5.830754065 | 11.78308353 |
| TO MIC | KPN_03336: 3652949-3653470 | 16 | 0.76376243 | 11.07003498 | 25.29418698 |
| CFT MIC | KPN_03336: 3652949-3653470 | 64 | 0.76363852 | 11.32836094 | 25.45706257 |
| P/T MIC | KPN_01766: 1947966-1948751 | 128 | 0.763460449 | 14.81949346 | 12.42014088 |
| CPE MIC | KPN_01182: 1331512-1332783 | 16 | 0.763288932 | 35.2128155 | 120.4810289 |
| CPE MIC | KPN_01762: 1944660-1945901 | 4 | 0.763104479 | 14.65996249 | 12.20397515 |
| LVX MIC | KPN_00957: 1081235-1081342 | 1 | 0.762745098 | 12.89952438 | 22.69865562 |
| AUG MIC | KPN_03336: 3652949-3653470 | 32 | 0.762211274 | 11.3458617 | 25.60940404 |
| AUG MIC | KPN_00957: 1081235-1081342 | 64 | 0.762206722 | 15.0494498 | 23.69042851 |
| IMP MIC | KPN_00957: 1081235-1081342 | 1 | 0.761889664 | 15.07749834 | 23.75253354 |
| CPE MIC | KPN_01772: 1953630-1954052 | 4 | 0.761821014 | 17.39048663 | 13.88210208 |
| CAX MIC | KPN_01793: 1975788-1977488 | 1 | 0.760522818 | 19.72497642 | 17.55192863 |
| CFT MIC | KPN_01793: 1975788-1977488 | 1 | 0.760522818 | 19.72497642 | 17.55192863 |
| CRM MIC | KPN_03336: 3652949-3653470 | 64.1 | 0.760179143 | 11.21858903 | 25.33689325 |
| CAX MIC | KPN_01762: 1944660-1945901 | 32 | 0.759685124 | 14.66688464 | 12.36657261 |
| AZT MIC | KPN_01765: 1947291-1947956 | 64 | 0.759580305 | 12.27090506 | 9.942012633 |
| CPE MIC | KPN_01776: 1957110-1958672 | 8 | 0.759123689 | 14.73158435 | 12.31603736 |
| CFT MIC | KPN_04540: 4971080-4973572 | 64 | 0.758928571 | 7.658256187 | 2.193267196 |
| AZT MIC | KPN_01772: 1953630-1954052 | 64.1 | 0.758913174 | 17.39571491 | 14.05148921 |
| AZT MIC | KPN_01766: 1947966-1948751 | 64.1 | 0.758524478 | 14.82873038 | 12.63669987 |
| A/S MIC | KPN_01776: 1957110-1958672 | 64.1 | 0.758339206 | 14.78733147 | 12.52676788 |
| AUG MIC | KPN_04540: 4971080-4973572 | 32 | 0.758000408 | 7.555273912 | 2.099521426 |
| ETP MIC | KPN_00957: 1081235-1081342 | 32 | 0.75792011 | 15.32688614 | 24.05211958 |
| CAX MIC | KPN_01790: 1972135-1973631 | 8 | 0.757658239 | 13.08164122 | 11.31584511 |
| CPE MIC | KPN_01790: 1972135-1973631 | 4 | 0.757507756 | 12.97237908 | 10.70547697 |
| CAZ MIC | KPN_01772: 1953630-1954052 | 64.1 | 0.757458544 | 17.38550538 | 13.9918728 |
| TO MIC | KPN_01765: 1947291-1947956 | 16 | 0.75745195 | 12.22861394 | 9.828459103 |
| CAX MIC | KPN_04540: 4971080-4973572 | 64 | 0.757383418 | 7.782320561 | 2.210951284 |
| AZT MIC | ygbJ: 1782199-1783101 | 64.1 | 0.757222547 | 12.28366179 | 29.14744037 |
| CAX MIC | KPN_01767: 1948966-1949805 | 16 | 0.75711194 | 14.47944984 | 12.36073696 |
| TO MIC | KPN_01773: 1954090-1955208 | 16 | 0.757047968 | 14.5282128 | 10.97269612 |
| CAZ MIC | KPN_01773: 1954090-1955208 | 64 | 0.756824926 | 14.5872474 | 11.02900629 |
| AZT MIC | KPN_00596: 673027-673989 | 8 | 0.756753663 | 23.08629939 | 20.58626307 |
| CRM MIC | KPN_01776: 1957110-1958672 | 64.1 | 0.756695968 | 14.75035459 | 12.42014088 |
| CP MIC | rmlC: 2715683-2716237 | 8.1 | 0.756545936 | 5.568497439 | 11.19348271 |
| A/S MIC | KPN_01790: 1972135-1973631 | 64.1 | 0.75628411 | 12.96633239 | 10.96759291 |
| CAX MIC | ygbK: 1780922-1782187 | 64 | 0.756188719 | 12.43233872 | 26.41227769 |
| CFT MIC | KPN_01767: 1948966-1949805 | 8 | 0.756059729 | 14.491735 | 12.40819316 |
| GM MIC | KPN_00957: 1081235-1081342 | 1 | 0.755993151 | 12.84408698 | 22.47979844 |
| CFT MIC | KPN_01762: 1944660-1945901 | 8 | 0.755836048 | 14.70148494 | 12.61333714 |
| AZT MIC | fim: 3725589-3728198 | 4 | 0.755702067 | 22.61675264 | 20.69081158 |
| CFT MIC | KPN_01790: 1972135-1973631 | 4 | 0.755601761 | 13.08905358 | 11.35575759 |
| AM MIC | KPN_00957: 1081235-1081342 | 128.1 | 0.755575648 | 12.44073167 | 22.25180254 |
| AZT MIC | dgoT: 4482049-4483386 | 64.1 | 0.755220715 | 18.74190417 | 16.7033483 |
| CAZ MIC | dgoT: 4482049-4483386 | 64 | 0.754973876 | 18.75909443 | 16.73958232 |
| CAX MIC | KPN_00596: 673027-673989 | 8 | 0.754690873 | 23.08937221 | 20.59706645 |
| CFT MIC | ygbK: 1780922-1782187 | 32 | 0.75402112 | 12.42445311 | 26.40289376 |
| TO MIC | KPN_01762: 1944660-1945901 | 16 | 0.753865355 | 14.64297977 | 12.40625523 |
| P/T MIC | KPN_01773: 1954090-1955208 | 64 | 0.75371268 | 14.54262135 | 10.92212932 |
| TO MIC | fim: 3729790-3730368 | 16 | 0.753582335 | 25.15081133 | 21.52357792 |
| ETP MIC | KPN_01182: 1331512-1332783 | 0.25 | 0.753482375 | 34.90281995 | 116.3716507 |
| CPE MIC | ygbK: 1780922-1782187 | 4 | 0.753436937 | 12.42684948 | 26.76489379 |
| GM MIC | KPN_01776: 1957110-1958672 | 1 | 0.753435501 | 14.91173446 | 12.92068423 |
| A/S MIC | KPN_04541: 4973659-4974345 | 64.1 | 0.753334446 | 10.18260317 | 2.040359952 |
| CPE MIC | KPN_01773: 1954090-1955208 | 4 | 0.753315803 | 14.5282128 | 10.86893977 |
| CFZ MIC | KPN_00596: 673027-673989 | 32.1 | 0.753261098 | 23.13875924 | 20.62136625 |
| TO MIC | KPN_00596: 673027-673989 | 4 | 0.75326087 | 23.11493217 | 20.5721784 |
| CFT MIC | KPN_01772: 1953630-1954052 | 32 | 0.753222246 | 17.38550538 | 14.19325741 |
| P/T MIC | stbA: 300128-300664 | 128 | 0.753189274 | 19.81584806 | 16.96721519 |
| AZT MIC | ygbK: 1780922-1782187 | 64.1 | 0.753172244 | 12.4301817 | 27.17968099 |
| TO MIC | KPN_01772: 1953630-1954052 | 16 | 0.752692562 | 17.35313626 | 14.19325741 |
| CFT MIC | ygbJ: 1782199-1783101 | 32 | 0.752690185 | 12.27307925 | 28.41434521 |
| AZT MIC | KPN_01773: 1954090-1955208 | 64.1 | 0.752448022 | 14.53571639 | 11.02900629 |
| CP MIC | KPN_01762: 1944660-1945901 | 4 | 0.752369164 | 14.65666635 | 12.35546733 |
| CP MIC | KPN_01767: 1948966-1949805 | 4 | 0.752230937 | 14.35342113 | 11.8106157 |
| GM MIC | KPN_00596: 673027-673989 | 2 | 0.752118522 | 23.0596253 | 20.56889713 |
| CAX MIC | fim: 3725589-3728198 | 2 | 0.751736111 | 22.61675264 | 20.69081158 |
| CFT MIC | fim: 3725589-3728198 | 2 | 0.751736111 | 22.61675264 | 20.69081158 |
| CAX MIC | KPN_01776: 1957110-1958672 | 8 | 0.751557745 | 14.89274787 | 12.93042875 |
| LVX MIC | KPN_01762: 1944660-1945901 | 4 | 0.751430942 | 14.66386574 | 12.42375859 |
| CFT MIC | KPN_00593: 669995-670945 | 4 | 0.751413044 | 15.9772972 | 14.36448194 |
| CPE MIC | ygbJ: 1782199-1783101 | 4 | 0.751169958 | 12.27710908 | 28.87713025 |
| CAZ MIC | KPN_00596: 673027-673989 | 8 | 0.750923913 | 23.11493217 | 20.65945949 |
| TE MIC | KPN_02951: 3243459-3243980 | 4 | 0.750770416 | 8.691988115 | 4.209890862 |
| CFT MIC | KPN_01776: 1957110-1958672 | 4 | 0.750631313 | 14.89274787 | 12.93042875 |
| GM MIC | KPN_01784: 1966562-1967182 | 1 | 0.750462474 | 18.03712077 | 12.69938622 |
| CAX MIC | KPN_01772: 1953630-1954052 | 32 | 0.750412836 | 17.39376789 | 14.59467291 |
| TO MIC | ygbJ: 1782199-1783101 | 16 | 0.750307637 | 12.30322131 | 28.87713025 |

In the two Examples with E. coli and K. pneumoniae on over 2,300 pathogenic bacteria that were compared we highlighted mutations in known drug targets and present novel putative genetic causes for resistance. Beyond single AA exchanges especially gene dosage effects seem to be of high importance for genetic resistance. Comparing both genera, we interestingly discovered identical AA mutations associated to drug resistance.

We investigated herein a genetic approach for the identification of drug-resistance genes and carefully compared these results to AST as the gold standard.

In the comprehensive study, we performed whole genome sequencing and ASTs for 1,162 E. coli and 1,176 K. pneumoniae isolates. We focused on pathogenic gram-negatives E. coli and K. pneumoniae, due to their multi-drug resistance and increasing frequency of causing severe bacteremia and sepsis. Choosing 21 drugs with indications for E. coli/K. pneumoniae enabled us to perform an elaborate analysis of the susceptibility of the clinical isolates. In total, we found 25,646 significant sites for E. coli and 40,896 significant sites for K. pneumoniae (p-value<10⁻⁹). Our method correctly identified several known gene/drug combinations: gyrA (Ciprofloxacin, Levofloxacin), parC (Ciprofloxacin, Levofloxacin), ampC (Cefalotin), folC (Trimethoprim Sulfamethoxaxole), mrcB (Cefazolin), pbpC (Cefazolin), pbpG (Ceftazidime), ftsI (Cefazolin), mrdA (Cefazolin), dacC (Ertapenem), dacB (Ertapenem, Meropenem), and mrcA (Ertapenem, Imipenem, Ceftazidime, Cefazolin) rendering this approach suitable for detecting these resistance mechanisms.

Besides the identification of single nucleotide variants that are statistically highly associated with drug resistance, we also found gene duplications and deletions as sporadic resistance mechanisms. In 23 cases for E. coli and 216 cases for K. pneumoniae, we see alterations in local sequence coverage as indicator of such structural changes in both genomes. While for membrane or transporter proteins both an increase or a decrease of gene dosage can influence drug susceptibility by not allowing the drug to permeate the membranes or to more efficiently transport it out of the cell, a decrease of the quantity of metabolic enzymes or transcription factors is not as easily interpretable in this context, and might be related to the fitness of the isolates. Interestingly, we discovered not only mutations in the β-lactamase SHV-11 (KPN_01607) for K. pneumoniae, but also identified dosage effects for this gene with increased coverage for resistant bacteria. Since it seems that SHV-type resistance genes are ubiquitous in K. pneumoniae, point mutations could lead to an improved activity of this enzyme. In addition, many Klebsiella also possess plasmids encoding for β-lactamases. Both, an altered activity as well as an increased expression of lactamases would lead to an improved resistance to lactams. Mendonca et al. (Mendonca, N., Ferreira, E., Louro, D., Participants, A. & Canica, M. Molecular epidemiology and antimicrobial susceptibility of extended- and broad-spectrum beta-lactamase-producing Klebsiella pneumoniae isolated in Portugal. Int J Antimicrob Agents 34, 29-37 (2009)) also identified the AA positions 234 and 235 as mutated in β-lactamases SHV-73 and SHV-107, but with other AA exchanges than we found in SHV-11. Because of the intrinsic β-lactamase activity, K. pneumoniae strains consequently exhibit low-level resistance to β-lactam compounds. This is also visible in the many positions that we identified that were significantly associated with Ampicillin, although the high number of resistance against lactams may cover other resistances.

Since both E. coli and K. pneumoniae belong to the group of Enterobacteriaceae, we tried to identify AA mutations that occur in both strains in functionally similar proteins and being associated with resistance to drugs in our analysis. We found 55 mutations in homologous proteins at the exact same AA position. This might give additional insights into the evolutionary development of resistances between these related strains and might represent novel putative drug targets.

Another source of information that might improve the accuracy of our analysis are the strain-specific plasmids. Mapping the sequencing data against those plasmids will extend our knowledge about additional resistance mechanisms. In a first approach, we mapped a subset of the E. coli sequencing data to about 300 E. coli plasmids. Among the genes having the highest mutation rates were repA1, trbI, psiB, and traG that are directly involved in replication, plasmid transfer, and maintenance and might play an indirect role in resistance development by giving its host the ability to facilitate spreading of resistance genes.

A genetic test for the combined pathogen identification and antimicrobial susceptibility testing direct from the patient sample can reduce the time-to actionable result significantly from several days to hours, thereby enabling targeted treatment. Furthermore, this approach will not be restricted to central labs, but point of care devices can be developed that allow for respective tests. Such technology along with the present methods and computer program products could revolutionize the care, e.g. in intense care units or for admissions to hospitals in general. Furthermore, even applications like real time outbreak monitoring can be achieved using the present methods.

Instead of using only single variants, a combination of several variant positions can improve the prediction accuracy and further reduce false positive findings that are influenced by other factors.

Compared to methods relying on MALDI-TOF MS, our genetic approach has the advantage that it covers almost the complete genome and thus enables us to identify the potential genomic sites that might be related to resistance. While MALDI-TOF MS can also be used to identify point mutations in bacterial proteins, this technology only detects a subset of proteins and of these not all are equally well covered. In addition, the identification and differentiation of certain related strains is not always feasible. Our genetic method allows for covering almost the whole genome and compute a best breakpoint for the separation of isolates into resistant and susceptible groups.

Compared to approaches using MALDI-TOF MS, the present approach has the further advantage that, as it covers almost the complete genome, enables us to identify the potential genomic sites that might be related to resistance. While MALDI-TOF MS can also be used to identify point mutations in bacterial proteins, this technology only detects a subset of proteins and of these not all are equally well covered. In addition, the identification and differentiation of certain related strains is not always feasible.

The present method allows computing a best breakpoint for the separation of isolates into resistant and susceptible groups. The inventors designed a flexible software tool that allows to consider—besides the best breakpoints—also values defined by different guidelines (e.g. European and US guidelines), preparing for an application of the GAST in different countries.

The invention further demonstrates that the present approach is capable of identifying mutations in genes that are already known as drug targets, as well as detecting potential new target sites.

Studies that combine whole genome sequencing with substantial culture based susceptibility tests allow for the first time a genome-wide association between genotype and drug resistance. As for human genome wide association studies (GWAS), substantial cohorts are however required. Respective approaches for pathogenic bacteria enable better and more personalized utilization of current antimicrobial drugs. Beyond this, the improved understanding of genetic resistance mechanisms can also promote the development of novel drugs, targeting these mechanisms.

We demonstrate that next generation sequencing combined with AST in a genome wide association study is capable of identifying mutations in genes that are already known as drug targets, as well as detecting potential new resistance mechanisms and respectively drug target sites. According to our results, gene dosage effects also play a key role for drug resistance. The here presented pipeline can be easily applied to investigate the genetic resistance of other gram-negative bacteria such as Pseudomonas species and also to gram-positive bacteria such as Staphylococcus aureus.

The current approach enables
a. Identification and validation of markers for genetic identification and susceptibility/resistance testing within one diagnostic test
b. validation of known drug targets and modes of action
c. detection of potentially novel resistance mechanisms leading to putative novel target/secondary target genes for new therapies

1. A diagnostic method of determining an infection of a patient with Klebsiella species potentially resistant to antimicrobial drug, e.g. antibiotic, treatment, comprising the steps of:
a) obtaining or providing a sample containing or suspected of containing at least one Klebsiella species from the patient;
b) determining the presence of at least one mutation in at least two genes from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, wherein the presence of said at least two mutations is indicative of an infection with an antimicrobial drug, e.g. antibiotic, resistant Klebsiella strain in said patient.

2. A method of selecting a treatment of a patient suffering from an infection with a potentially resistant Klebsiella strain, comprising the steps of:
a) obtaining or providing a sample containing or suspected of containing at least one Klebsiella species from the patient;
b) determining the presence of at least one mutation in at least two genes from the group of genes consisting of parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and KPN_04195, and/or KOX_26125, KOX_13365, KOX_16735, KOX_25695, KOX_12270, and KOX_15055, wherein the presence of said at least two mutations is indicative of a resistance to one or more antimicrobial, e.g. antibiotic, drugs;
c) identifying said at least one or more antimicrobial, e.g. antibiotic, drugs; and
d) selecting one or more antimicrobial, e.g. antibiotic, drugs different from the ones identified in step c) and being suitable for the treatment of a Klebsiella infection.

3. The method of one or more of the preceding claims, wherein the Klebsiella species is Klebsiella pneumoniae and at least a mutation in parC, particularly in position 3763210 with regard to reference genome NC_009648 as annotated at the NCBI, is determined, and/or
wherein the Klebsiella species is Klebsiella oxytoca and at least a mutation in KOX_26125, particularly in position 5645611, with regard to reference genome NC_016612 as annotated at the NCBI, is determined.

4. The method of one or more of the preceding claims, wherein the method involves determining the resistance of Klebsiella to one or more antimicrobial, e.g. antibiotic, drugs.

5. The method of any one of claims 1 to 4, wherein the antimicrobial, e.g. antibiotic, drug is selected from lactam antibiotics and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195, and/or KOX_26125, KOX_13365, KOX_16735, KOX_25695, KOX_12270, and/or KOX_15055; and/or
wherein the antimicrobial, e.g. antibiotic, drug is selected from quinolone antibiotics, particularly fluoroquinolone antibiotics, and/or polyketide antibiotics, particularly tetracycline antibiotics, and/or benzene derived/sulfonamide antibiotics, and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195, and/or KOX_26125; and/or wherein the antimicrobial, e.g. antibiotic, drug is selected from aminoglycoside antibiotics and the presence of a mutation in the following genes is determined: parC, KPN_01607, gyrA, KPN_02451, baeR, aceF, ybgH, ynjE, KPN_01951, KPN_01961, KPN_02114, mhpA, KPN_02128, KPN_02144, KPN_02149, ydiJ, btuE, oppC, pth, KPN_02298, KPN_02302, dadA, yoaA, ftn, cbl, hisB, yegQ, yehY, KPN_02580, yejH, KPN_02621, yfaW, KPN_02170, KPN_02025, livG, livM, livH, fliY, yedQ, abgB, treA, baeS, KPN_02399, ydcR, anmK, ccmF, KPN_02440, KPN_02540, KPN_01752, and/or KPN_04195.

6. The method of one or more of the preceding claims, wherein the antimicrobial drug, e.g. antibiotic drug, is selected from the group consisting of Amoxicillin/K Clavulanate (AUG), Ampicillin (AM), Aztreonam (AZT), Cefazolin (CFZ), Cefepime (CPE), Cefotaxime (CFT), Ceftazidime (CAZ), Ceftriaxone (CAX), Cefuroxime (CRM), Cephalotin (CF), Ciprofloxacin (CP), Ertapenem (ETP), Gentamicin (GM), Imipenem (IMP), Levofloxacin (LVX), Meropenem (MER), Piperacillin/Tazobactam (P/T), Ampicillin/Sulbactam (A/S), Tetracycline (TE), Tobramycin (TO), and Trimethoprim/Sulfamethoxazole (T/S).

7. The method of any one of claims 1 to 6, wherein resistance to Klebsiella pneumoniae is determined, the antibiotic drug is at least one of CF, CFT, IMP, CFZ, CRM, ETP, CAX, AZT, P/T, CPE, AM, A/S, CAZ, MER, AUG, CP, LVX, GM, TO, TE, and T/S and a mutation in at least one of the following nucleotide positions is detected with regard to reference genome NC_009648: 3763210, 1784305, 1784302, 2905411, 2673906, 2773232, 140517, 809148, 1364586, 2150691, 2159024, 2317024, 2325877, 2331649, 2347930, 2355785, 2365629, 2375692, 2402871, 2459360, 2517274, 2521829, 2532012, 2547536, 2629283, 2658497, 2703286, 2774521, 2812941, 2831238, 2875745, 2878878, 2920245, 2379716, 2218319, 2504346, 2505230, 2506816, 2641631, 2646728, 1704769, 2524562, 2772839, 2627362, 2124017, 2174754, 2275805, 2662814, 2784148, 1933723, 4595554; and/or
wherein resistance to Klebsiella oxytoca is determined, the antibiotic drug is at least one of CF, CFZ, CRM, AZT, AM, and A/S and a mutation in at least one of the following nucleotide positions is detected with regard to reference genome NC_016612: 5645611, 2887469, 2887473, 3631990, 5544665, 5544668, 2652345, 3260573; and/or
wherein resistance to Klebsiella oxytoca is determined, the antibiotic drug is at least one of CFT, CAX, P/T, CPE, CAZ, AUG, CP, LVX, TE, and T/S and a mutation in at least one of the following nucleotide positions is detected with regard to reference genome NC_016612: 5645611.

8. The method of any one of claims 1 to 7, wherein the resistance of a bacterial microorganism belonging to the species Klebsiella against 1, 2, 3, 4, 5, 6, 7, 8, 9, 10, 11, 12, 13, 14, 15 or 16, 17, 18, 19, 20 or 21 antibiotic drugs is determined.

9. The method of one or more of the preceding claims, wherein determining the nucleic acid sequence information or the presence of a mutation comprises determining a partial sequence or an entire sequence of the at least two genes.

10. The method of one or more of the preceding claims, wherein determining the nucleic acid sequence information or the presence of a mutation comprises determining a partial or entire sequence of the genome of the Klebsiella species, wherein said partial or entire sequence of the genome comprises at least a partial sequence of said at least two genes.

11. The method of one or more of the preceding claims, wherein determining the nucleic acid sequence information or the presence of a mutation comprises using a next generation sequencing or high throughput sequencing method, preferably wherein a partial or entire genome sequence of the bacterial organism of Klebsiella species is determined by using a next generation sequencing or high throughput sequencing method.

12. A method of determining an antimicrobial drug, e.g. antibiotic, resistance profile for bacterial microorganisms of Klebsiella species, comprising:
obtaining or providing a first data set of gene sequences of a plurality of clinical isolates of Klebsiella species;
providing a second data set of antimicrobial drug, e.g. antibiotic, resistance of the plurality of clinical isolates of Klebsiella species;
aligning the gene sequences of the first data set to at least one, preferably one, reference genome of Klebsiella, and/or assembling the gene sequence of the first data set, at least in part;
analyzing the gene sequences of the first data set for genetic variants to obtain a third data set of genetic variants;
correlating the third data set with the second data set and statistically analyzing the correlation; and
determining the genetic sites in the genome of Klebsiella associated with antimicrobial drug, e.g. antibiotic, resistance.

13. A diagnostic method of determining an infection of a patient with Klebsiella species potentially resistant to antimicrobial drug treatment, comprising the steps of:
a) obtaining or providing a sample containing or suspected of containing a bacterial microorganism belonging to the species Klebsiella from the patient;
b) determining the presence of at least one mutation in at least one gene of the bacterial microorganism belonging to the species Klebsiella as determined by the method of claim 12, wherein the presence of said at least one mutation is indicative of an infection with an antimicrobial drug resistant Klebsiella strain in said patient.

14. A method of selecting a treatment of a patient suffering from an infection with a potentially resistant Klebsiella strain, comprising the steps of:
a) obtaining or providing a sample containing or suspected of containing a bacterial microorganism belonging to the species Klebsiella from the patient;
b) determining the presence of at least one mutation in at least one gene of the bacterial microorganism belonging to the species Klebsiella as determined by the method of claim 12, wherein the presence of said at least one mutation is indicative of a resistance to one or more antimicrobial drugs;
c) identifying said at least one or more antimicrobial drugs; and
d) selecting one or more antimicrobial drugs different from the ones identified in step c) and being suitable for the treatment of a Klebsiella infection.

15. A method of acquiring an antimicrobial drug, e.g. antibiotic, resistance profile for bacterial microorganisms of Klebsiella species, comprising:
obtaining or providing a first data set of gene sequences of a clinical isolate of Klebsiella species;
providing a second data set of antimicrobial drug, e.g. antibiotic, resistance of a plurality of clinical isolates of Klebsiella species;
aligning the gene sequences of the first data set to at least one, preferably one, reference genome of Klebsiella, and/or assembling the gene sequence of the first data set, at least in part;
analyzing the gene sequences of the first data set for genetic variants to obtain a third data set of genetic variants of the first data set;
correlating the third data set with the second data set and statistically analyzing the correlation; and
determining the genetic sites in the genome of Klebsiella of the first data set associated with antimicrobial drug, e.g. antibiotic, resistance.

16. Computer program product comprising computer executable instructions which, when executed, perform a method according to any one of claims 12 to 15.
